(19) United States
(12) Patent Application Publication
(10) Pub. No.: US 2024/0165257 A1
(43) Pub. Date: May 23, 2024

(54) ANTI-GUCY2C ANTIBODY AND USES THEREOF

(71) Applicant: Heidelberg Pharma Research GmbH, Ladenburg (DE)

(72) Inventors: Torsten HECHLER, Ladenburg (DE); Andreas PAHL, Ladenburg (DE); Stephanie VOSS, Ladenburg (DE); Christian ORLIK, Ladenburg (DE)

(73) Assignee: Heidelberg Pharma Research GmbH, Ladenburg (DE)

(21) Appl. No.: 18/498,631

(22) Filed: Oct. 31, 2023

(30) Foreign Application Priority Data
Nov. 1, 2022 (EP) 22204963.7

(51) Int. Cl. A61K 47/68; A61P 35/00; C07K 16/40

(52) U.S. Cl. CPC A61K 47/6871; A61K 47/6831; A61K 47/6889; A61P 35/00; C07K 16/40; C07K 2317/52

(57) ABSTRACT

In a first aspect the present application pertains to an anti-GUCY2C antibody, corresponding antibody-drug conjugates comprising amatoxin for use in the treatment of gastrointestinal cancers, such as colorectal cancer, and pancreatic cancer. In a second aspect the present invention pertains to pharmaceutical compositions comprising the antibody-drug conjugates of the invention for use in the treatment of gastrointestinal cancer. The present invention also pertains to methods of treatment of gastrointestinal cancer using the antibody-drug conjugates of the invention.

### FIELD OF THE INVENTION

The present application relates to anti-GUCY2C antibodies and corresponding antibody-drug conjugates comprising an amatoxin. In a further aspect, the invention relates to pharmaceutical compositions comprising such conjugates for use in the treatment of gastrointestinal cancer, such as colorectal cancer (CRC), or metastatic colorectal cancer (mCRC).

### BACKGROUND

Gastrointestinal cancers account for approximately 26% of all cancer diagnoses and are responsible for approximately 35% of cancer deaths worldwide. There were an estimated 4.8 million new cases of gastrointestinal (GI) cancers and 3.4 million related deaths, worldwide, in 2018.

Gastrointestinal cancers comprise esophageal, gastric, colorectal, liver, esophgeal and pancreatic tumors, whereby colocrectal cancer (CRC) is most frequent and contributes to about 10.2% of all new cases, followed by gastric cancer (5.7%), liver cancer (4.7%), esophageal cancer (3.2%) and pancreatic cancer (2.5%). CRC contributes to about 9% of all cancer related deaths, followed by gastric cancer (8.2%), liver cancer (8.2%), esphageal cancer (5.3%) and pancreatic cancer (4.5%) with about 3.4 million GI cancer deaths globally in 2018 (Arnold et al. (2020) Gastroenterology 159:335-349). Colorectal cancer (CRC) is thus, the third most common cancer worldwide after lung and breast cancer but the second leading cause of cancer death.

In women CRC is the second most common adult cancer and the third most common in men, and it is the fourth leading cause of cancer death.

The 5-year and 10-year survival rates are 65% and 58%, respectively, and incidence and mortality rates are 25% higher in men than in women. From 1975 to 2013, CRC incidence rate increased from 10-15 cases per 100,000 population of Americans between the age of 20-49 years of age. This is paralleled by an increase in CRC cases of about 20% in developing countries such as Argentina, Brazil, and China. Furthermore, 30% of all patients with CRC experience metastasis for which the prognosis still remains poor with a median 5-year survival of only 18.5% in the United States and 27.7% in Europe.

Despite the fact that the incidence rate has declined over the past 30 years due to improvements in screening and treatment paradigms it is alarming, that incidence rates in individuals younger than 50 years have been steadily increasing, with a 2.2% annual overall incidence rate rise between 2012-2016. The exact etiology for this paradigm shift is unknown, however, it is likely that this attributable to changes in diet and other lifestyle risk factors, or may be attributable to increased nonsystematic screening of young adults.

10%-20% of all patients with CRC possess a positive family history and ˜5% of all cases of CRC are linked to a known hereditary CRC syndrome detectable by germline. In some cases, increased sporadic CRC incidence is associated with longstanding inflammatory bowel disease and variable lifestyle factors such as physical inactivity, unhealthy diet, smoking, obesity, and heavy alcohol consumption.

Differences in clinical outcomes and drug responsiveness of CRC treatment are dependent on the location of cancer along the colon and rectum. Relevant contributing factors include their distinct physiological functions, gut microbiome, regionally resident immune cell types, dietary carcinogens, timing of disease detection. In addition, ontogeny factors may contribute to disease severity and treatment outcome:

The proximal (right) large intestine (cecum, ascending colon, and the transverse colon) derives from the embryonic midgut, whereas the distal (left) large intestine (splenic flexure, descending colon, sigmoid colon, and rectum) derives from the embryonic hindgut. These ontogenetic differences are associated with differential gene expression patterns along the proximal-distal axis. In women and in African-Americans, proximal sporadic colon tumors, are more frequently diagnosed with an incidence of 51%-62% of cases which also show a higher TNM stage at first diagnosis, display a pattern with high levels of genome-wide promoter hypermethylation referred to as CpG island methylator phenotype (CIMP), exhibit microsatellite instability (MSI) due to deficient DNA mismatch repair mechanisms (dMMR), are more frequently mutated in KRAS and BRAF and have a worse prognosis in terms of survival. Distal colorectal tumors are more likely to present with chromosomal instability (CIN) and show a more favorable prognosis.

Genomic profiling of CRC has revealed significant intratumoral and intertumoral heterogeneity resulting from the accumulation of genetic mutations and chromosomal aberrations during disease initiation and progression. Genomic instability in CRC presents as one of two major forms: CIN and MSI. CRC lacking CIN or MSI is classified as genome stable (GS) CRC.

In GS CRC, the DNA repair genes and tumor suppressors are likely to be transcriptionally silenced through CIMP, though a large proportion of MSI CRCs and a small population of CIN CRCs are also CIMP-positive, and about 10% of CRCs are negative for CIN, MSI, or CIMP.

Chromosome instability (CIN) CIN is characterized by chromosomal numerical alterations (aneuploidy) and structural alterations (somatic copy number alterations, deletions, insertions, amplifications, or loss of heterozygosity), occurring in 65%-70% of sporadic CRCs. Nearly all CIN tumors show activated Wnt signaling, and 80% harbor mutational inactivation of APC, a negative regulator of the Wht pathway. Mutational inactivation/deletion of TP53 occurs in 60% of CIN tumors and p53 loss of function directly drives CIN and provides a permissive context for genome instability mechanisms.

With respect to genome instability, combined telomere dysfunction and p53 deficiency is a major CIN mechanism, as revealed by the occurrence of anaphase bridges in early-stage carcinomas of human CRC and spontaneous CRC occurrence in telomerase-deficient p53 mutant mice.

Microsatellite instable (MSI) CRC is characterized by the presence of microsatellites, DNA sequences which containing repetitive motifs that tend to accumulate higher mutation rates than other genomic regions. MSI is the phenotypic manifestation of dMMR resulting from mutational inactivation of MMR genes, including MLH1, MSH2, MSH3, MSH6, PMS2 and Exo1 (De'Angelis et al. Acta Biomed 2018 Dec. 17; 89(9-S): 97-101).

Other important factors that contribute to the etiology of CRC are somatic genetic alterations which activate key signal transduction pathways that promote the proproliferative state of CRC cancer cells. In CRC, the major proproliferative signaling pathways are the EGFR-RAS and WNT-β-catenin pathways.

EGFR signaling EGFR activation triggers downstream RAS/RAF/MEK/ERK and PI3K/AKT signaling cascades which ultimately lead to proliferation. Mutations in EGFR itself are rare in CRC with 1% of cases of CRC being attributable to a mutation in CRC (Barber et al N Engl J Med 351: 2883) and instead shows overexpression in ˜80% of CRCs. Enhanced EGFR activation can occur via post-translational modifications involving methylation of R198 and R200 by protein arginine methyltransferase 1 (PRMT1), which enhances its binding to EGF and consequent signaling activation, even in the presence of the EGFR inhibitor.

It has recently been demonstrated that hepatocyte growth factor (HGF), the ligand of the MET-receptor, can fully replace EGF in the outgrowth of single Lgr5+ mouse intestinal stem cells (ISCs) to intestinal organoids, comprising all differentiated intestinal cell lineages. In addition, HGF and EGF were equally effective in promoting expansion of Apc-mutant mouse organoids, while Met deletion in ISCs in vivo attenuated stem-cell fitness and the formation of Apc-driven intestinal adenomas. These findings indicate that EGFR and MET signaling have overlapping and partially redundant, functionality in the mouse intestinal mucosa. Recent results also indicate that HGF/MET signaling can fully overcome EGFR inhibition in human ISCs and CRC stem cells, allowing expansion of single normal ISCs, as well as APC-mutant cells into organoids, even in the presence of EGFR-inhibition (Joosten et al Gastroenterology 2019 October; 157(4):1153-1155).

Another factor that is decisive for the clinical effectiveness of EGFR blockade is the mutational status of its downstream signaling components, specifically, gain-of-function mutations in RAS, RAF, MEK, or ERK, which can maintain cancer cell proliferation and survival upon EGFR inhibition. Activating mutations in KRAS, NRAS, or HRAS are collectively present in ˜50% of cases of CRC; these mutations involve codons 12 or 13 and, less frequently, codons 61, 117, or 146. With respect to the RAS pathway in CRC, the RAS effector, BRAF, is mutated in 10%-15% of early-stage CRC and around 5% in stage IV CRC, in the hotspotcodon 600 (V600E). Clinically, the inhibition of BRAF alone showed limited activity in metastatic BRAFV600E CRC owing to EGFR-mediated reactivation of MAPK signaling leading to the approval of combinations of EGFR inhibitors with BRAF and MEK inhibitors that have now become the standard of care in patients with metastatic BRAF mutant tumors.

Screening for CRC can be done via either direct visualization method such as colonoscopy, CT colonography, flexible sigmoidoscopy, flexible sigmoidoscopy with fecal immunochemical test (FIT) or via stool-based tests such as guaiac-based fecal occult blood test, FIT, or multi-targeted stool DNA test or via serology test such ad SEPT9 DNA test.

Once the diagnosis of CRC is made, staging is an important factor as it determines treatment options for the patient. For tumor staging the Tumor, node, metastasis (TNM) system from the combined American Joint Committee on Cancer (AJCC)/Union for International Cancer Control (UICC) is a commonly used staging system which defines the following cancer stages: Standard conventional treatments for CRC are surgery, chemotherapy and radiotherapy. Depending on the localization and progression of the disease, these treatments can be used in combination. Treatment options include for example for stage I surgical resection alone. For stage Ill curative surgery is followed with adjuvant chemotherapy as standard of care. Total mesorectal excision (TME) through laparoscopic and transanal surgery approaches are often the options for localized cancer and whenever the tumor location is easy to access. However, complete removal of all cancer cells is often not possible. About 66% of stage II and and 61% of stage III colon and rectal patients have to undergo further treatments with adjuvant chemotherapy and/or radiotherapy. These treatments have many side effects due to their unspecificity and cytotoxicity toward any cells that are growing and dividing. Despite recent improvements in the diagnostic and treatment options, 54% of patients relapse even after neoadjuvant treatment. Thus, it is crucial to have more alternative and effective treatments to treat CRC patients.

Current chemotherapy for CRC includes both single-agent therapy, which is mainly fluoropyrimidine (5-FU)-based, and multipleagent regimens containing one or several drugs, including oxaliplatin (OX), irinotecan (IRI), and capecitabine (CAP or XELODA or XEL), whereby the combined therapy regimens FOLFOX (5-FU+OX), FOXFIRI (5-FU+IRI), XELOX or CAPOX (CAP+OX), and CAPIRI (CAP+OX) represent the main approaches in first-line treatment. However, chemotherapy is associated with certain limitations, such as systemic toxicity, unsatisfying response rate, as well as unpredictable innate and acquired resistance, and low tumor-specific selectivity.

Patients with metastatic colorectal cancer (mCRC) have a 5-year overall survival rate of <10%. Currently, the main chemotherapy agents used for the treatment of the affected patients are fluoropyrimidine (intravenous or oral)-based as either single-agent treatments, such as intravenous 5-fluorouracil (5-FU) and oral capecitabine (CAP) or as multiple-agent regimens, including the combinations FOLFOX (5-FU and oxaliplatin), FOLFIRI (5-FU and irinotecan), XELOX/CAPOX (CAP and oxaliplatin), CAPIRI (CAP and irinotecan). Beyond traditional chemotherapy, the EGFR-targeted agents, cetuximab and panitumumab, are approved for first-line treatment of mCRC. Ccombinations of cetuximab with FOLFIRI or panitumumab with FOLFOX have significantly enhanced therapeutic efficacy. However, treatment decisions for early-stage mCRC do not consider BRAF or KRAS mutations, given the dramatically poor prognosis conferred by these mutations as seen in clinical trials. Although the survival rate of patients with mCRC has improved in recent years, the response and prognosis of patients with the aforementioned mutations are still poor.

The current lack of effective therapies and non-specific cytotoxic limitations have led investigators to transition towards the development of predictive, preventative, and personalized medicine strategies to improve the treatment of CRC and mCRC given the substantial unmet need for prospective therapies for patients with BRAF- or KRAS-mutant mCRC.

Gastric cancer is the second leading cause of death from malignant disease worldwide, with especially high mortality rates in East, South, and Central Asia; Central and Eastern Europe; and South America. Treatment options for gastric cancer vary with cancer stages: Very early cancer can typically be treated by surgery. Potentially resectable cancer may typically be treated by surgery as first treatment, with either subtotal gastrectomy or total gastrectomy. Nearby lymph nodes and possibly parts of nearby organs may be removed as well. In addition, patients may receive chemotherapy alone or chemo plus radiation therapy (chemoradiation), for example patients may receive 5-fluorouracil (5-FU) in combination with cisplatin (CDDP) (FP therapy). Other possible treatment regimens include epirubicin, cisplatin, and fluorouracil (ECF therapy), or regimens in which fluorouracil was replaced by capecitabine (ECX therapy), or ECF therapy in which cisplatin is replaced by oxaliplatin (EOF therapy) may be used. Alternatively, both cisplatin and fluorouracil can be replaced by oxaliplatin and capecitabine (EOX therapy).

In metastatic gastric cancers treatment is aimed at controlling the growth of the cancer and may include chemotherapy alone, chemotherapy plus immunotherapy, or chemotherapy along with radiation therapy. The combination of a platinum and fluoropyrimidine (5-FU) is the global standard first-line chemotherapy regimen within a non-curative setting. After platinum and 5-FU failure paclitaxel plus ramucirumab has been established as standard second-line therapy. However, treatment-related neuropathy, progression during or rapid recurrence following perioperative FLOT regimen (fluorouracil, oxaliplatin, docetaxel) have raised the demand for a taxane-free second-line therapy. Trifluridine/tipiracil has recently been approved for patients with metastatic gastric cancer. In addition to chemotherapy, patients may be treated with immune checkpoint inhibitors, such as ipilimumab (anti-CTLA-4), nivolumab (anti-PD-1), pembrolizumab (anti-PD-1), or atezolizumab (anti-PD-L1).

Diagnostically gastrointestinal endoscopy is indispensable for the diagnosis of gastric cancer, as well as staging laparoscopy (SL), which is a minimally invasive, brief procedure that only requires a small incision. The advantages of SL include providing an accurate diagnosis of peritoneal dissemination and extraserosal invasion, and the ability to perform peritoneal lavage for cytology. In patients with advanced gastric cancer for whom imaging does not yield a diagnosis, peritoneal lavage cytology obtained before treatment can be very important for treatment planning. Peritoneal lavage cytology obtained by SL for evaluation of peritoneal dissemination is thought to be useful for assessing the effects of neoadjuvant chemotherapy and/or immunotherapy.

The most frequent liver tumor is hepatocellular carcinoma (HCC) among all primary liver cancers, accounting for 75%-85% of cases. HCC is usually diagnosed at an advanced stage, for which there remain limited effective treatment options. Until 2007, there were no effective treatment options for patients diagnosed with advanced-stage disease or patients who transitioned into advanced-stage disease after other treatments failed. Sorafenib was the first systemic drug approved by the U.S. Food and Drug Administration (FDA) as standard treatment for advanced HCC between 2007 and 2016. In recent years other small molecule drugs have been deleveloped which include lenvatinib, regorafenib, or cabozantinib which have either been approved or are still undergoing clinical trials. These drugs are currently also tested in combination with immune checkpoint inhibitors, such as nivolumab, or pembrolizumab. In addition, combinations of immune checkpoint inhibitors and angiogenesis inhibitors, such as atezolizumab and bevacizumab are being tested.

Esophageal cancer as one form of GI cancer is being treated according to its stage, which may include endoscopic treatments, as well as chemotherapy and radiation therapy. Common drugs and drug combinations that are being used to treat esophageal cancer but are usually not given with radiation include ECF: epirubicin (Ellence), cisplatin, and 5-FU (especially for gastroesophageal junction tumors), DCF: docetaxel (Taxotere), cisplatin, and 5-FU, or trifluridine and tipiracil (Lonsurt), a combination drug in pill form.

For pancreatic cancer several chemotherapy regimens have been approved the most widely used and best-studied agent is Gemcitabine. Gemcitabine is often administered in combination with albumin-bound (Nab) Paclitaxel, which improved survival time compared to Gemcitabine monotherapy. Alternative treatment options include the multi-drug regimen FOLFIRINOX (5-Fluorouracil, Leucovorin, Irinotecan, and Oxaliplatin). Also, for pancreatic cancer, combination therapies using immune checkpoint inhibitors such as pemprolizumab, or nivolumab in combination with gemcitabine, nab-paclitaxel, or capecitabine are being clinically assessed.

Guanylyl Cyclase 2C (GUCY2C, EC: 4.6.1.2) is a member of a family of receptor-enzyme proteins synthesizing guanosine 3′,5′-cyclic monophosphate (cyclic GMP: cGMP). GUCY2C is a transmembrane receptor for the endogenous hormonal ligands:guanylin and uroguanylin. Ligand binding to the extracellular receptor catalyzes the conversion of GTP into cyclic GMP (cGMP) and initiates downstream cGMP-related signaling pathways, which are implicated in the regulation of intestinal homeostatic processes such as epithelial cell proliferation, differentiation, and apoptosis (Lisby et al. Expert Rev Precis Med Drug Dev. 2021; 6(2): 117-129).

GUCY2C is specifically expressed by intestinal epithelial cells. GUCY2C regulates the dynamic progression of cells along the crypt-villus and crypt-surface axis, coordinating homeostatic processes including proliferation, DNA repair, metabolic programming, lineage-specific cell fate, and epithelial-nesenchymal interactions organizing that axis. Given the major role of GUCY2C in maintaining epithelial regeneration, dysregulation of the GUCY2C-cGMP axis promotes pathologies that include inflammatory bowel disease and bowel transit disorder, in addition to colorectal cancer. Importantly, silencing the GUCY2C signaling axis is associated with colorectal tumorigenesis through the loss of ligand binding.

GUCY2C protein can be detected near-universally (>95%) in all primary and metastatic human colorectal tumors regardless of anatomical location or grade, as well as a subset of gastroesophageal and pancreatic tumors, but not in tumors arising outside the GI tract. GUCY2C was also shown to be expressed in esophageal, gastric and pancreatic tumors (Danaee et al (2017) PLoS One. 2017; 12(12): e0189953).

Given its unique expression pattern in CRC and mCRC various approaches have been taken to utilize GUCY2C in the treatment of these diseases which have included cancer vaccines using GUCY2C, its use as a target for CAR-T cell therapy (Magee et al. Cancer Immunol Res. 6(5), 509-516 (2018)), or the use of therapeutic antibodies and antibody-drug conjugates targeting GUCY2C. WO2013/163633 A1 discloses anti-GUCY2C antibodies and anti-GUCY2C ADCs comprising auristatin E (MMAE) and auristatin F MMAE (MMAF) which inhibit mitosis by inhibiting tubulin polymerization. Clinical trials of the anti-GUCY2C ADC TAK264 (MLN0264, 5F9vcMMAE) which employed MMAE as payload have been terminated. WO2021/205325 A1 discloses anti-CD3-GUCY2C bispecific antibodies and respective use in cancer therapy to induce a cytolytic T cell response in GUCY2C positive target cells.

These efforts indicate that GUCY2C-directed therapies may be effective, however, given the lack of approved GUCY2C-directed therapies there is still a considerable need for therapeutic modalities with high potency and a good efficacy profile.

### SUMMARY OF THE INVENTION

The inventors surprisingly found that the amatoxin-based conjugates according to the present invention comprising anti-GUCY2C antibodies, or antigen-binding antibody fragments thereof in combination with a non-cleavable or cleavable linker linking the anti-GUCY2C antibody, or antibody fragment to an amatoxin as disclosed herein, were characterized by a high potency and efficacy in vitro, as well as in vivo models of GUCY2C-positive cancers.

In view of the prior art, it was hence one object of the present invention to provide antibodies, which specifically bind to GUCY2C, as well as conjugates comprising said anti-GUCY2C antibodies which comprise at least one amatoxin and at least one linker linking said anti-GUCY2C antibody with said at least one amatoxin, that mediate cytotoxic effects in target cells, as described in the present application.

It was one further object of the present invention to provide a pharmaceutical composition comprising such conjugates.

It was one further object of the present invention to provide compounds for use in methods for treatment of cancer.

In a further object the present invention pertains to the provision of a composition comprising the conjugates of the invention as disclosed herein and at least one immune checkpoint inhibitor for use in the treatment of cancer, particularly for use in the treatment of colorectal cancer.

### DESCRIPTION OF THE FIGURES

FIG. 1. Markush structure of various amatoxins. The numbers in bold type (1 to 8) designate the standard numbering of the eight amino acids forming the amatoxin. The standard designations of the atoms in amino acids 1, 3, and 4 are also shown (Greek letters a to γ, Greek letters a to 8, and numbers from 1′ to 7′, respectively).

FIG. 2. In vitro cytotoxicity of anti-GUCY2C-ADCs of the invention comprising conjugate (XV) assayed on HEK293 cells stably expressing human GUCY2C.

FIG. 3 In vitro cytotoxicity of anti-GUCY2C-ADCs on HEK cells stably expressing human GUCY2C. (A) cytoxtoxicity of anti-GUCY2C-ADCs conjugated to conjugate (XII), (B) cytoxtoxicity of anti-GUCY2C-ADCs conjugated to conjugate (XIV). Cytotoxicity assays indicate that the anti-GUCY2C ADCs tested display a similar cytotoxic potential in the picomlar range on HEK293-GUCY2C cells.

FIG. 4. Efficacy of the anti-GUCY2C conjugates mAb1-LALA-D265C-(XIV) and mAb8-LALA-D265C-(XIV) in a subcutaneous tumor model using HEK293-GUCY2C-(HDP)-2B3 tumor cells in female NOD Scid mice after single dose intravenous application of ADCs as indicated. The results indicate that a single dose application of the ADCs at concentrations of 1.25 mg/kg i.v. and 2.5 mg/kg i.v. result in effective suppression of tumor growth.

FIG. 5. Kaplan-Meier plots of survival for the subcutaneous GUCY2C-tumor model shown in FIG. 4 indicating enhanced survival of animals in groups 2, 3, 5 and 6.

FIG. 6. Liver toxicity of anti-GUCY2C conjugates of the invention in cynomolgus monkeys. Effect of administration of the anti-GUCY2C-ADC of the invention comprising heavy and light chain amino acid sequences SEQ ID NO: 85 and SEQ ID NO: 87 on liver enzymes alanine aminotransferase (ALT), aspartate aminotransferase (AST) and lactate dehydrogenase (LDH). The results indicate that liver enzymes ALT, AST and LDH are transiently elevated upon administration of the conjugates of the invention and reach baseline levels within 2-3 weeks of administration of the conjugates. The results further indicate a maximum tolerated dose of 2 mg/kg for conjugates comprising the linker payload (XIV) and 5 mg/kg for conjugates comprising the linker payload (XII).

FIG. 7. Treatment of murine patient-derived xenograft (PDX) models of colorectal cancer using conjugates of the invention. (A) PDX model of a primary KRAS mutant (G13D) adenocarcinoma stage T3NOMx treated with conjugates of the invention comprising linker-payload conjugate (XII), (XIV) as indicated. (B) PDX model of a primary adenocarcinoma stage T3NOMO treated with conjugates of the invention comprising linker-payload conjugate (XII), (XIV) as indicated. Conjugates in (A), (B) were administered as single dose, or repeated dosing once every week for 4 weeks (“q7dx4”).

FIG. 8. Sequence alignment of (A) heavy chain variable regions (B) light chain variable regions of the antibodies of the invention. The alignment was done using the Clustal W algorithm (Thompson et al, Nucleic Acids Res. 1994 Nov. 11; 22(22):4673-80). CRDs of both V_{H} and V_{L} sequences are indicated by boxes.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values. Recitation of ranges of values herein is merely intended to serve as a shorthand method of referring individually to each separate value falling within the range. Unless otherwise indicated herein, each individual value is incorporated into the specification as if it were individually recited herein.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions. The definitions of the chemical groups as used herein shall have the meaning and be defined as provided in “Compendium of Chemical Terminology” (“Gold Book”) published by the International Union of Pure and Applied Chemistry (IUPAC) version 2.3.3, goldbook.iupac.org, ISBN: 0-9678550-9-8), the content of which is hereby incorporated by reference.

Throughout this application the term “about” is used which shall refer to +/−10% of the numerical value with which it is used.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions.

According to a first aspect of the present invention, an isolated antibody or antibody fragment is provided which specifically binds to guanylyl cyclase C (GUCY2C), wherein the antibody or antibody fragment comprises a heavy chain variable region (V_{H}) which comprises a framework region 1 (FR_{H}1), complementarity-determining region 1 (CDR_{H}1), a framework region (FR_{H}2), a complementarity-determining region 2 (CDR_{H}2), a framework region 3 (FR_{H}3), a complementarity-determining region 3 (CDR_{H}3), and a framework region 4 (FR_{H}4), wherein
FR_{H}1 comprises an amino acid sequence selected from SEQ ID NOs: 33, 40, 45, 51;
CDR_{H}1 comprises an amino acid sequence selected from SEQ ID NOs: 34, 41, 46, 52;
FR_{H}2 comprises an amino acid sequence according to SEQ ID NO: 35; 55;
CDR_{H}2 comprises an amino acid sequence selected from SEQ ID Nos: 36, 42, 47; 120
FR_{H}3 comprises an amino acid sequence selected from SEQ ID NOs: 37, 43, 48, 53;
CDR_{H}3 comprises an amino acid sequence selected from SEQ ID Nos: 38, 44, 49; 54
FR_{H}4 comprises an amino acid sequence selected from SEQ ID NOs: 39, 50.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or V_{H}) and a heavy chain constant region (abbreviated as CH or C_{H}). The heavy chain constant region is comprised of three domains, namely CH1, CH2 and CH3. Each light chain contains a light chain variable region (abbreviated as VL or V_{L}) and a light chain constant region (abbreviated as CL or C_{L}). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen. The constant regions (Fc regions) are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement activation via the C1q component, leading to the chemotactic, opsonic and, potentially in the case of a viable cellular antigen target, cytolytic actions of complement. Human antibodies of the IgG1 class are the most potent in activating the complement system and are therefore the desirable isotype for the therapeutic application of the antibodies of the present invention.

Human Fcγ receptors include FcγR (1), FcγRIIa, FcγRIIb, FcVRIIIa and neonatal FcRn for which it was demonstrated that a common set of IgG1 residues is involved in binding all FcγRs, while FcγRII and FcγRIII utilize distinct sites outside of this common set (Shields et al. (2001) J. Biol. Chem 276: 6591-6604). One group of IgG1 residues reduced binding to all FcgRs when altered to alanine: Pro-238, Asp-265, Asp-270, Asn-297 and Pro-239 (numbering according to EU numbering system). All are in the IgG CH2 domain and clustered near the hinge joining CH1 and CH2. While FcγR1 utilizes only the common set of IgG1 residues for binding, FcγRII and FcγRIII interact with distinct residues in addition to the common set. Alteration of some residues reduced binding only to FcγRII (e.g. Arg-292) or FcγRIII (e.g. Glu-293). Some variants showed improved binding to FcγRII or FcγRIII but did not affect binding to the other receptor. The neonatal FcRn receptor is believed to be involved in both antibody clearance and the transcytosis across tissues (see: Junghans (1997) Immunol. Res 16: 29-57; and Ghetie et al. (2000) Annu. Rev. Immunol. 18: 739-766). Human IgG1 residues determined to interact directly with human FcRn includes Ile253, Ser254, Lys288, Thr307, Gln311, Asn434 and His435.

The terms “CDR”, “CDRL1”, “CDR_{L}2”, “CDR_{L}3”, “CDR_{H}1”, “CDR_{H}2”, “CDR_{H}3” as used herein follow the Kabat numbering convention (Kabat et al., Sequences of Proteins of Immunological Interest, 4th Ed., U.S. Department of Health and Human Services, National Institutes of Health (1987)). However, although the Kabat numbering convention for amino acid residues in variable domain sequences and full length antibody sequences is used throughout this specification, it will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues based on the numbering system used are considered part of the CDR sequence and would be understood to be so by a skilled person, however, these differences functionally do not imply altered or different antigen-binding of the respective antibody. Other numbering conventions for CDR sequences available to a skilled person include “AbM” (University of Bath) and “contact” (University College London) methods.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸M or less, even more preferably 5×10⁻¹⁰ M or less, and most preferably from about 10⁻⁸, 10⁻⁹ M to about 10⁻¹⁰ M, 10⁻¹¹ or less, or e.g. from about 10⁻¹⁰ M to about 10⁻¹¹ M or less, but does not bind to unrelated (e.g. structurally or sequence unrelated) antigens with an affinity equal to the affinity for the specific target.

The antibody of the invention which may e.g. also be referred to as immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice.

The term “antibody fragment” or “antigen-binding fragment” as used herein refers to an antibody fragment or analog of an antibody which retains the binding specificity of the parent anti-GUCY2C antibody as disclosed herein and comprises a portion (for example, one or more CDRs) or variable region of the antigen binding region of the parent antibody. The antibody fragment is, for example, Fab, Fab′, F(ab′)₂, Fv fragment, sc-Fv, unibody, diabody, linear antibody, nanobody, domain antibody, or multispecific antibody fragment formed from the antibody fragment. A Fab fragment consists of the CH1 and variable regions of one light chain and one heavy chain. The heavy chain of a Fab molecule cannot form a disulfide bond with another heavy chain molecule. A Fab′ fragment as contains a light chain and a portion of a heavy chain that contains the VH domain, the CH1 domain, and the region between the CH1 and CH2 domains. A F(ab′)₂ fragment contains two light chains and two heavy chains containing a portion of the constant region between the CH1 and CH2 domains, thereby forming an interchain disulfide bond between the two heavy chains. A F(ab′)₂ fragment is composed of two Fab′ fragments held together by the disulfide bond between the two heavy chains. A “Fv fragment” contains variable regions from both the heavy and light chains, but lacks the constant region. The term “single-chain antibody” is a single-chain recombinant protein formed by connecting the heavy chain variable region VH and the light chain variable region VL of an antibody through a connecting peptide. It is the smallest antibody fragment with a complete antigen-binding site. The term “domain antibody fragment” is an immunoglobulin fragment with immunological functions that only contains a heavy chain variable region or a light chain variable region chain. In some cases, two or more VH regions are covalently linked to a peptide linker to form a bivalent domain antibody fragment. The two VH regions of the bivalent domain antibody fragment can target the same or different antigens.

According to one embodiment, the anti-GUCY2C antibody or antibody fragment of the invention as disclosed herein comprises a light chain variable region (V_{L}) which comprises a framework region 1 (FR_{L}1), complementarity-determining region (CDR_{L}1), a FR_{L}2, a CDR_{L}2, a FR_{L}3, a CDR_{L}3, and a FR_{L}4, wherein
FR_{L}1 comprises an amino acid sequence selected from SEQ ID NOs: 1, 13, 20, 27;
CDR_{L}1 comprises an amino acid sequence selected from SEQ ID NOs: 2, 14, 21, 28;
FR_{L}2 comprises an amino acid sequence selected from SEQ ID NOs: 3, 15, 29
CDR_{L}2 comprises an amino acid sequence selected from SEQ ID NOs: 4, 16, 23, 30;
FR_{L}3 comprises an amino acid sequence selected from SEQ ID Nos 5, 8, 17, 24, 31;
CDR_{L}3 comprises an amino acid sequence selected from SEQ ID NOs: 6, 9, 11, 18, 25, 32;
FR_{L}4 comprises an amino acid sequence selected from SEQ ID NOs: 7, 10, 12, 19, 26.

According to one embodiment, the anti-GUCY2C antibody of the invention comprises a heavy chain variable region (V_{H}) which comprises a framework region 1 (FR_{H}1), complementarity-determining region 1 (CDR_{H}1), a FR_{H}2, a CDR_{H}2, a FR_{H}3, a CDR_{H}3, and a FR_{H}4, wherein
FR_{H}1 comprises an amino acid sequence according to SEQ ID NO: 33
CDR_{H}1 comprises an amino acid sequence according to SEQ ID NOs: 34,
FR_{H}2 comprises an amino acid sequence according to SEQ ID NO: 35;
CDR_{H}2 comprises an amino acid sequence according to SEQ ID NO: 36;
FR_{H}3 comprises an amino acid sequence according to SEQ ID NO: 37;
CDR_{H}3 comprises an amino acid sequence according to SEQ ID NO: 38;
FR_{H}4 comprises an amino acid sequence according to SEQ ID NO: 39, and

wherein the antibody comprises a light chain variable region (V_{L}) which comprises a framework region 1 (FR_{L}1), complementarity-determining region (CDR_{L}1), a FR_{L}2, a CDR_{L}2, a FR_{L}3, a CDR_{L}3, and a FR_{L}4, wherein
FR_{L}1 comprises an amino acid according to SEQ ID NO: 1; 121
CDR_{L}1 comprises an amino acid according to SEQ ID NOs: 2;
FR_{L}2 comprises an amino acid according to SEQ ID NOs: 3, 15, 29
CDR_{L}2 comprises an amino acid sequence selected from SEQ ID NO: 4;
FR_{L}3 comprises an amino acid sequence selected from SEQ ID Nos: 5, 8;
CDR_{L}3 comprises an amino acid sequence selected from SEQ ID NOs: 6, 9, 11;
FR_{L}4 comprises an amino acid sequence selected from SEQ ID NOs: 7, 10, 12.

According to one embodiment the anti-GUCY2C antibody or antibody fragment of the invention comprises a heavy chain variable region (V_{H}) which comprises a framework region 1 (FR_{H}1), complementarity-determining region (CDR_{H}1), a FR_{H}2, a CDR_{H}2, a FR_{H}3, a CDR_{H}3, and a FR_{H}4, wherein
FR_{H}1 comprises an amino acid sequence according to SEQ ID NO: 40, 45, 51;
CDR_{H}1 comprises an amino acid sequence according to SEQ ID NOs: 41, 46, 52,
FR_{H}2 comprises an amino acid sequence according to SEQ ID NO: 35; 55
CDR_{H}2 comprises an amino acid sequence according to SEQ ID NO: 42, 47;
FR_{H}3 comprises an amino acid sequence according to SEQ ID NO: 43, 48, 53;
CDR_{H}3 comprises an amino acid sequence according to SEQ ID NO: 49, 44, 54
FR_{H}4 comprises an amino acid sequence according to SEQ ID NO: 50, 39 and

wherein the antibody comprises a light chain variable region (V_{L}) which comprises a framework region 1 (FR_{L}1), complementarity-determining region (CDR_{L}1), a FR_{L}2, a CDR_{L}2, a FR_{L}3, a CDR_{L}3, and a FR_{L}4, wherein
FR_{L}1 comprises an amino acid according to SEQ ID NO: 13, 20, 27;
CDR_{L}1 comprises an amino acid according to SEQ ID NOs: 14, 21, 28;
FR_{L}2 comprises an amino acid according to SEQ ID NOs: 15, 22, 29;
CDR_{L}2 comprises an amino acid sequence selected from SEQ ID NO: 16, 23, 30;
FR_{L}3 comprises an amino acid sequence selected from SEQ ID Nos: 17, 24, 31;
CDR_{L}3 comprises an amino acid sequence selected from SEQ ID NOs: 18, 25; 32
FR_{L}4 comprises an amino acid sequence selected from SEQ ID NOs: 19, 26.

According to one embodiment, the anti-GUCY2C antibody of the invention as disclosed herein comprises a light chain variable region (V_{L}) which comprises a CDR_{L}1 according to SEQ ID NO: 2, a CDR_{L}2 according to SEQ ID NO: 4, a CDR_{L}3 according to SEQ ID NO: 9 and a heavy chain variable region (V_{H}) which comprises a CDR_{H}1 according to SEQ ID NO: 34, a CDR_{H}2 according to SEQ ID NO: 36, a CDR_{H}3 according to SEQ ID NO: 38.

According to one embodiment, the anti-GUCY2C antibody of the invention as disclosed herein comprises a light chain variable region (V_{L}) which comprises a CDR_{L}1 according to SEQ ID NO: 2, a CDR_{L}2 according to SEQ ID NO: 4, a CDR_{L}3 according to SEQ ID NO: 6 and a heavy chain variable region (V_{H}) which comprises a CDR_{H}1 according to SEQ ID NO: 34, a CDR_{H}2 according to SEQ ID NO: 36, a CDR_{H}3 according to SEQ ID NO: 38.

According to one embodiment, the anti-GUCY2C antibody of the invention as disclosed herein comprises a light chain variable region (V_{L}) which comprises a CDR_{L}1 according to SEQ ID NO: 2, a CDR_{L}2 according to SEQ ID NO: 4, a CDR_{L}3 according to SEQ ID NO: 11 and a heavy chain variable region (V_{H}) which comprises a CDR_{H}1 according to SEQ ID NO: 34, a CDR_{H}2 according to SEQ ID NO: 36, a CDR_{H}3 according to SEQ ID NO: 38.

According to one embodiment, the anti-GUCY2C antibody of the invention as disclosed herein comprises a light chain variable region (V_{L}) which comprises a CDR_{L}1 according to SEQ ID NO: 28, a CDR_{L}2 according to SEQ ID NO: 30, a CDR_{L}3 according to SEQ ID NO: 32 and a heavy chain variable region (V_{H}) which comprises a CDR_{H}1 according to SEQ ID NO: 52, a CDR_{H}2 according to SEQ ID NO: 120, a CDR_{H}3 according to SEQ ID NO: 54.

According to one embodiment, the anti-GUCY2C antibody of the invention as disclosed herein comprises a light chain variable region (V_{L}) which comprises a CDR_{L}1 according to SEQ ID NO: 21, a CDR_{L}2 according to SEQ ID NO: 23, a CDR_{L}3 according to SEQ ID NO: 25 and a heavy chain variable region (V_{H}) which comprises a CDR_{H}1 according to SEQ ID NO: 46, a CDR_{H}2 according to SEQ ID NO: 47, a CDR_{H}3 according to SEQ ID NO: 49.

According to one embodiment, the anti-GUCY2C antibody of the invention as disclosed herein comprises a light chain variable region (V_{L}) which comprises a CDR_{L}1 according to SEQ ID NO: 14, a CDR_{L}2 according to SEQ ID NO: 16, a CDR_{L}3 according to SEQ ID NO: 18 and a heavy chain variable region (V_{H}) which comprises a CDR_{H}1 according to SEQ ID NO: 41, a CDR_{H}2 according to SEQ ID NO: 42, a CDR_{H}3 according to SEQ ID NO: 44.

In one embodiment, light chain variable region (V_{L}) of the anti-GUCY2C antibody of the invention comprises the amino acid sequence according to SEQ ID NOs. 2; 3; 4, 121 and SEQ ID NOs: 8; 9; 10 and a heavy chain variable region (V_{H}) which comprises the amino acid sequences according to SEQ ID NOs: 33; 34; 35; 36; 37; 38; 39 (e.g. mAb #1).

In one embodiment, light chain variable region (V_{L}) of the anti-GUCY2C antibody of the invention comprises the amino acid sequence according to SEQ ID NOs. 1-7 and a heavy chain variable region (V_{H}) which comprises the amino acid sequences according to SEQ ID NOs: 33; 34; 35; 36; 37; 38; 39 (e.g. mAb #8).

In one embodiment, light chain variable region (V_{L}) of the anti-GUCY2C antibody of the invention comprises the amino acid sequence according to SEQ ID NOs. 2; 3; 4; 5, 11, 12, 121 and a heavy chain variable region (V_{H}) which comprises the amino acid sequences according to SEQ ID NOs: 33; 34; 35; 36; 37; 38; 39 (e.g. mAb #24).

In one embodiment, light chain variable region (V_{L}) of the anti-GUCY2C antibody of the invention comprises the amino acid sequence according to SEQ ID NOs. 20; 21; 22; 23; 24; 25; 26 and a heavy chain variable region (V_{H}) which comprises the amino acid sequences according to SEQ ID NOs: 45, 46, 48, 49, 50 and 55 (e.g. mAb #28).

In one embodiment, light chain variable region (V_{L}) of the anti-GUCY2C antibody of the invention comprises the amino acid sequence according to SEQ ID NOs. 40, 41, 55, 42, 43, 44, 39 and a heavy chain variable region (V_{H}) which comprises the amino acid sequences according to SEQ ID NOs: 13; 14; 15; 16; 17; 18; 19 (e.g. mAb #40).

In one embodiment, light chain variable region (V_{L}) of the anti-GUCY2C antibody of the invention comprises the amino acid sequence according to SEQ ID NOs. 27; 28; 29; 30; 32; 19 and a heavy chain variable region (V_{H}) which comprises the amino acid sequences according to SEQ ID NOs: 51: 52; 55; 120; 53; 54; and 39 (e.g. mAb #41).

According to some embodiments, the anti-GUCY2C antibody or antibody fragment according to the invention comprises the subsequent combinations of heavy chain variable region (V_{H}) and light chain variable region (V_{L}) comprising amino acid sequences according to
SEQ ID NO: 62, SEQ ID NO: 56;
SEQ ID NO: 63, SEQ ID NO: 57
SEQ ID NO: 64, SEQ ID NO: 58
SEQ ID NO: 65, SEQ ID NO: 59
SEQ ID NO: 66, SEQ ID NO: 60
SEQ ID NO: 67, SEQ ID NO: 61.

Each of the V_{L} and V_{H} combinations disclosed above may e.g. also comprise a V_{L}, or a V_{H}, or both V_{L} and V_{H} which share at least 90%, 95% sequence identity with SEQ ID Nos:56, 57, 58, 59, 60, 61, 62, 63, 64, 65, 66, or 67, each VL, VH sequence independently, or both VL and VH sequences together, provided that the corresponding anti-GUCY2C antibody or antibody fragment specifically binds to the same epitope on human or cynomolgus GUCY2C. Epitope mapping for the antibodies of the invention may e.g. be done by hydrogen deuterium exchange (HDX) in analogy to the methods disclosed in Huang et al. MAbs 2018 January; 10(1): 95-103, or e.g. as reviewed in Jethva and Gross, Front. Anal. Sci., 18 May 2023

According to one embodiment, the antibody, or antibody fragment according to the present invention can be a monoclonal antibody. As used herein, the term “monoclonal antibody” (“mAb”) refers to a preparation of antibody molecules of single binding specificity and affinity for a particular epitope, representing a homogenous antibody population, i.e., a homogeneous population consisting of a whole immunoglobulin, or a fragment. Preferably the monoclonal anti-GUCY2C antibody of the invention as disclosed herein is of the IgG isotype, e.g. IgG1, or IgG4, more preferably of the IgG1 isotype.

Monoclonal antibodies (mAb) derived from e.g. mouse may cause unwanted immunological side-effects when administered to humans due to the fact that they contain a protein from another species which may elicit antibodies. In order to overcome this problem, antibody humanization and maturation methods have been designed to generate antibody molecules with minimal immunogenicity when applied to humans, while ideally still retaining specificity and affinity of the non-human parental antibody (for review see Almagro and Fransson 2008).

Thus, according to preferred embodiments, the anti-GUCY2C antibody of the invention is a humanized or human antibody. The term “humanized” antibody as used herein refers to an antibody that contains minimal sequences derived from non-human immunoglobulin. Thus, “humanized” forms of non-human (e.g., murine) antibodies are chimeric antibodies that contain minimal sequence derived from the non-human antibody. All or substantially all of the framework regions may also be those of a human immunoglobulin sequence. The humanized antibody may also contain at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and 6,180,370. The human antibodies, such as the human anti-GUCY2C antibody of the invention, can be made by the hybridoma method using human myeloma or mouse-human heteromyeloma cells lines see Kozbor (1984) J. Immunol 133, 3001. Alternative methods include the use of phage libraries or transgenic mice both of which utilize human variable region repertories (see Winter (1994) Annu. Rev. Immunol 12: 433-455; Green (1999) J. Immunol. Methods 231: 11-23). Several strains of transgenic mice are now available wherein their mouse immunoglobulin loci has been replaced with human immunoglobulin gene segments (see Tomizuka (2000) PNAS 97: 722-727; Fishwild (1996) Nature Biotechnol. 14: 845-851; Mendez (1997) Nature Genetics, 15:146-156). Upon antigen challenge such mice are capable of producing a repertoire of human antibodies from which antibodies of interest can be selected

According to some embodiments, the constant region of IgG heavy chain of the anti-GUCY2C IgG1 antibody of the invention can be selected from different allotypes. The term “allotype” in the context of the anti-GUCY2C antibody of the invention refers to inherited allelic variants arising from genetic differences between individuals which may be recognised as antigenic by members of the same species. For example, the antibody of the invention may have one of the allotypes G1m3, G1m17,1 or G1m17,1,2 allotypes or G1m(f), G1m(z,a), or G1m(z,a,x).

Human antibodies, such as e.g. the human anti-GUCY2C antibody of the invention, may comprise a kappa (κ) chain, which in humans is encoded by the immunoglobulin kappa locus (IGK) on chromosome 2 (locus: 2p11.2), or a lambda (A) chain, which in humans is encoded by the immunoglobulin lambda locus (IGL) on chromosome 22 (locus: 2q11.22). Accordingly, the anti-GUCY2C antibody of the invention may comprise two kappa light chains, or two lambda light chains comprising the VL sequences according to SEQ ID NO: 56, SEQ ID NO: 57, SEQ ID NO: 58, SEQ ID NO: 59, SEQ ID NO: 60, or SEQ ID NO: 61.

According to one embodiment, the anti-GUCY2C antibody, or antigen-binding fragment thereof of the invention can be glycosylated, or de-glycosylated, preferably the antibody of the invention is glycosylated and corresponding glycan can be an N-linked oligosaccharide chain at asparagine 297 of the heavy chain (numbering according to EU numbering system, Edelman et al., 1969, Proc Natl Acad Sci USA 63:78-85). Antibodies, as most glycoproteins, are typically produced as a mixture of glycoforms. This mixture is particularly apparent when antibodies are produced in eukaryotic, particularly mammalian cells. Each glycan moiety hat is added to the Fc-glycan such as e.g. fucose, bisecting GlcNAc, galactose and sialic acid, has been implicated in modulating antibody sensor affinity and/or antibody function. It is thus desirable to manufacture monoclonal antibodies which are characterized by a defined set of glycans to obtain antibodies with defined effector function. A variety of methods have been developed to manufacture defined glycoforms, see Zhang et al. (2004) Science 303: 371: Sears et al. (2001) Science 291: 2344; Wacker et al. (2002) Science 298: 1790; Davis et al. (2002) Chem. Rev. 102: 579; Hang et al. (2001) Acc. Chem. Res 34: 727. The anti-GUCY2C antibody of the invention as described herein preferably comprises a homogenous or defined number of glycoforms, e.g. from about 1, 2, 3, 4 to about 5, 6, 7, 8, or from about 5, 6, 7 to about 8, preferably 6 or less, e.g. 5, 4, 3 or less, more preferably two or one.

According to one embodiment, the anti-GUCY2C antibody, or the antigen-binding fragment thereof is a recombinant antibody or antigen-binding fragment thereof. The term “recombinant” or “recombinantly produced” as used herein refers to a protein, such as the anti-GUCY2C antibody or antigen-binding fragment of the invention as disclosed herein, that have been expressed in heterologous cells. For example, the anti-GUCY2C antibody or antigen-binding fragment of the invention may be expressed in prokaryotic or eukaryotic cells. Prokaryotic cells for the expression of the antibody of the invention include e.g. gram-negative bacteria such as E. coli, or gram-positive bacteria such as Bacillus subtilis. The use of heterologous prokaryotic expression systems may be particularly useful for the expression of antigen-binding fragments of the anti-GUCY2C antibody of the invention and may be done according to established protocols known in the art, such as e.g. Kwong and Rader, Curr. Protoc. Protein Sci. 55:6.10.1-6.10.14, for the expression of Fab framents.

The use of eukaryotic cells for the expression of the antibody or antigen-binding fragment of the invention is, however, preferred due to the glycosylation of the heterologously expressed antibody. It is even more preferred to use mammalian cells for the expression of the antibody or antigen-binding fragment of the invention. For example, Chinese hamster ovary (CHO) cells, or any of its genetically different progeny such as K1-, DukX B11-, DG44-cell lines may be used for the expression of the antibody of the invention. Other mammalian cell lines that may be used for the production of the antibody or antigen-binding fragment of the invention include e.g. NS0 cells, embryonic kidney (HEK) 293 cells, PER.C6 cells, MCF7 cells. Cells can e.g. be transfected with an expression vector comprising the coding sequence for the ati-GUCY2C antibody or antigen-binding fragment according to the invention. The expression vector or recombinant plasmid is produced by placing the coding antibody sequences under control of suitable regulatory genetic elements, including promoter and enhancer sequences like, e.g., a CMV promoter. Heavy and light chain sequences can e.g. be expressed from individual expression vectors which are co-transfected, or from dual expression vectors. Said transfection may be a transient transfection or a stabile transfection. The transfected cells are subsequently cultivated to produce the transfected antibody construct. When stabile transfection is performed, then stable clones secreting antibodies with properly associated heavy and light chains are selected by screening with an appropriate assay, such as, e.g., ELISA, subcloned, and propagated for future production. Corresponding methods are e.g. disclosed in WO03/018771.

According to one embodiment the anti-GUCY2C antibody of the invention comprises a Fc region which comprises the amino acid sequence according to SEQ ID NO: 68. Antibodies of the invention which comprise a Fc region comprising the amino acid sequence according SEQ ID NO: 68 are able to induce antibody-dependent cell-mediated cytotoxicity (ADCC), antibody-dependent cellular phagocytosis (ADCP), or complement-dependent cytotoxicity (CDC).

It may, however, be desirable to reduce or eliminate effector function of the antibody of the invention as disclosed herein for example to prevent target cell death, unwanted cytokine secretion, or killing of cells that express the Fcγ receptor such as macrophages.

Accordingly, the anti-GUCY2C antibody or antigen-binding fragments of the invention as described herein may also include modifications and/or mutations that alter the properties of the antibodies and/or fragments, such as those which decrease ADCC, ADCP, or complement-dependent cytotoxicity CDC as known in the art. Preferably, ADCC, ADCP and CDC are reduced by at least 90% or more, more preferably by at least 95%, more preferably by at least 97.5%, even more preferred by at least 98%, 99% compared to an antibody comprising a wildtype Fc region comprising e.g. the amino acid sequence according to SEQ ID NO: 68.

The binding of IgG1 to activating and inhibitory Fcγ receptors (FcγRs) or the first component of complement (C1q) depends on residues located in the hinge region and the CH2 domain. Two regions of the CH2 domain are critical for FcγRs and complement C1q binding, and have unique sequences. Substitution of human IgG1 and IgG2 residues at positions 233-236 and IgG4 residues at positions 327, 330 and 331 greatly reduced ADCC and CDC (Armour, et al., Eur. J. Immunol. 29(8) (1999) 2613-2624; Shields, et al., J. Biol. Chem. 276(9) (2001) 6591-6604, WO 2021/234402 A2).

Accordingly, in one embodiment, the anti-GUCY2C antibody, or antigen-binding fragment thereof as disclosed herein, comprises a variant Fc region, wherein said variant Fc region comprises at least one amino acid modification relative to a wild-type Fc region, such that said molecule has a reduced affinity for IgG1 Fc receptors FcγRI, FcγRII and FcγRIII as well as to complement component C1q compared to a wild-type Fc region (e.g. comprising an amino acid sequence according to SEQ ID NO: 68).

Affinity to an Fc region, such as the binding of IgG1 to FcγRs, can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay as disclosed in e.g. Wilkinson et al. PLoS One. 2021; 16(12): e0260954 or other mechanism of kinetics-based assay (e.g., BIACORE™, analysis or Octet™ analysis (forteBIO)), and other methods such as indirect binding assays, competitive binding assays fluorescence resonance energy transfer (FRET), gel electrophoresis and chromatography (e.g., gel filtration).

Thus, the anti-GUCY2C antibody as described herein have been genetically engineered to comprise a variant Fc region which comprise modification of at least one amino acid residue that directly contacts FcγRs based on structural and crystallographic analysis. The term “genetically engineered” or “genetic engineering” as used herein relates to the modification of the amino acid sequence or part thereof of a given or natural polypeptide or protein, such as e.g. the Fc region of an antibody, in the sense of nucleotide and/or amino acid substitution, insertion, deletion or reversion, or any combinations thereof, by gene technological methods, such as, e.g., site-directed mutagenesis as described in Carter, Biochem. J. (1986) Vol. 237: 1-7. As used herein, the term “amino acid substitution” or “mutation” relates to modifications of the amino acid sequence of the protein, wherein one or more amino acids are replaced with the same number of different amino acids, producing a protein which contains a different amino acid sequence than the original protein. A conservative amino acid substitution is understood to relate to a substitution which due to similar size, charge, polarity and/or conformation does not significantly affect the structure and function of the protein. Groups of conservative amino acids in that sense represent, e.g., the non-polar amino acids Gly, Ala, Val, Ile and Leu; the aromatic amino acids Phe, Trp and Tyr; the positively charged amino acids Lys, Arg and His; and the negatively charged amino acids Asp and Glu.

Depending on the intended use of the antibody such as e.g. in the manufacture of an antibody-drug conjugate, the Fc region of said antibody may further comprise at least one cysteine amino acid substitution at sites where the engineered cysteines are available for conjugation but do not perturb immunoglobulin folding and assembly. Corresponding cysteine-substituted or cysteine-engineered antibodies have disclosed in VVN2016040856A2, or Junutula, et al., 2008 Nature Biotech., 26(8):925-932; Doman et al (2009) Blood 114(13):2721-2729; U.S. Pat. Nos. 7,521,541; 7,723,485; VN02009/052249 and VYN2016/142049). The preferred cysteine substitution in the Fc region of the inventive antibodies as disclosed herein is D265C (according to EU numbering system) as disclosed in WO2016142049A1.

In one embodiment, the anti-GUCY2C antibody of the invention comprises an Fc region, or a fragment thereof, which comprises at least one amino acid substitution selected from from L234A, L234S, L234G, L235A, L235G, L235S, L235T, G236R, D265C, whereby the amino acid numbering is according to EU numbering system. The EU numbering system may also be referred to as “EU index as in Kabat” and refers to the numbering of the human IgG1 EU antibody, which refers to the numbering of the EU antibody of Edelman et al., 1969, Proc Nati Acad Sci USA 63:78-85.

In some embodiments, the anti-GUCY2C antibody of the invention comprises an Fc region which comprises the above amino acid substitutions singly or in combination, e.g. two or more (e.g. 2, 3, or 4) of the above amino acid substitutions as set out below:

| Fc amino acid substitution | Amino acid sequence |
|---|---|
| (according to EU numbering system) | according to |
| D265C | SEQ ID NO: 70 |
| L234A, L235A | SEQ ID NO: 69 |
| L234A, L235A, D265C | SEQ ID NO: 71 |
| L234A, L235A, G236R, D265C | SEQ ID NO: 72 |
| L234G, L235S, G236R, D265C | SEQ ID NO: 73 |
| L234S, L235T, G236R, D265C | SEQ ID NO: 74 |
| L234S, L235G, G236R, D265C | SEQ ID NO: 75 |

According to preferred embodiments the anti-GUCY2C antibody of the invention comprises a Fc region which comprises at least three amino acid subsitutions as disclosed hereinabove, preferably the Fc region comprises at least the amino acid subsitutions L234A, L235A, D265C (SEQ ID NO:71). The use of said Fc regions comprising at least the subsitutions L234A, L235A and D265C in the inventive anti-GUCY2C antibodies is e.g. particularly advantageous to reduce the interaction of a Fc region comprising said mutations with FcγRs by at least 95%, 97.5%, 99% compared to a wild-type Fc region while at the same time allowing site-specific thiol-based conjugation of linker-payloads, e.g. such as those disclosed herein. Corresponding use of such cysteine substituted Fc regions and antibodies comprising such mutations for linker-payload conjugation is e.g. disclosed in WO2016142049A1.

In some embodiments, the inventive anti-GUCY2C antibodies comprises an Fc region which comprises or consists of an amino acid sequence according to any of SEQ ID NO: 72, SEQ ID NO: 73, SEQ ID NO: 74, or SEQ ID NO: 75. The use of said Fc regions comprising four mutations may e.g. be advantageous to eliminate residual interaction of the Fc region with FcγRs thereby yielding in an Fc silenced antibodies of the invention.

In some embodiments the anti-GUCY2C antibody of the invention may thus comprise the following pairing of light chains (LC) with heavy chains (HC) as disclosed herein which comprise at least one, preferably at least three of the mutations as disclosed herein:

| Light chain (LC) | |
|---|---|
| amino acid sequence | Heavy chain (HC) amino acid sequence |
| SEQ ID NO: 78 | SEQ ID NOs: 79, 80, 81, 82, 83, or 84. |
| SEQ ID NO: 85 | SEQ ID NO: 86, 87, 88, 89, 90, or 91 |
| SEQ ID NO: 92 | SEQ ID NO: 93, 94, 95, 96, 97, or 98 |
| SEQ ID NO: 99 | SEQ ID NO: 100, 101, 102, 103, 104 or 105 |
| SEQ ID NO: 106 | SEQ ID NO: 107, 108, 109, 110, 111, or 112 |
| SEQ ID NO: 113 | SEQ ID NO: 114, 115, 116, 117, 118, or 119 |

According to preferred embodiments, the anti-GUCY2C antibody of the invention comprises a light chain and a heavy chain selected from

LC according to SEQ ID NO: 78, HC according to SEQ ID NO: 80, or

LC according to SEQ ID NO: 85, HC according to SEQ ID NO: 87, or

LC according to SEQ ID NO: 92, HC according to SEQ ID NO: 94, or

LC according to SEQ ID NO: 99, HC according to SEQ ID NO: 101, or

LC according to SEQ ID NO: 106, HC according to SEQ ID NO: 108, or

LC according to SEQ ID NO: 113, HC according to SEQ ID NO: 115.

According to more preferred embodiments, the anti-GUCY2C antibody of the invention comprises a light chain and a heavy chain selected from

LC according to SEQ ID NO: 78, HC according to SEQ ID NO: 80, or

LC according to SEQ ID NO: 85, HC according to SEQ ID NO: 87, or

LC according to SEQ ID NO: 92, HC according to SEQ ID NO: 94.

According to one embodiment the anti-GUCY2C antibody of the invention comprises a heavy chain variable region and alight chain variable region, wherein the heavy chain variable region of said antibody comprises a CDR_{H}1, a CDR_{H}2, and a CDR_{H}3 selected from SEQ ID NOs: 34, 41, 46, 52; 36, 42, 47; 38, 44, or 49; and wherein the framework regions FR_{H}1, FR_{H}2, FR_{H}3, FR_{H}4 of said antibody share at least 80%, 85%, preferably 90%, or 95% sequence similarity to any of SEQ ID NOs: 33, 40, 45, 51; 35; 55; 37, 43, 48, 53; 39, or 50.

The terms “sequence similarity”, “sequence identity”, or “similar in sequence” are used interchangeably throughout the present invention refer to the similarity or identity of two or more amino acid or polynucleotide sequences to each other or to a reference sequence. Sequence identity according to the invention may e.g. be determined over the whole length of each of the sequences being compared to a respective reference sequence (so-called “global alignment”), that is particularly suitable for sequences of the same or similar length, or over shorter, defined lengths (so-called “local alignment”), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. For example, to obtain an amino acid sequence having a sequence of at least 95% identity to a query amino acid sequence, up to 5% (5 of 100) of the amino acid residues in the subject sequence may be inserted or substituted with another amino acid or deleted.

Methods for comparing the identity and sequence similarity of two or more sequences are well known in the art. The percentage to which two sequences are identical can for example be determined by using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 83, 63-98; Pearson and Lipman (1988), Proc. Natl. Acad. Sci. U.S.A 85, 2444-2448). Sequences which are identical to other sequences to a certain extent can be identified by these programs. Furthermore, programs available in the Wisconsin Sequence Analysis Package (Devereux et al, 1984, Nucleic Acids Res., 387-395; Womble Methods Mol Biol. 2000; 132:3-22), for example the programs BESTFIT and GAP, may be used to determine the % identity between two polypeptide sequences. BESTFIT uses the “local sequence similarity” algorithm of (Smith and Waterman (1981), J. Mol. Biol. 147, 195-197) and finds the best single region of similarity between two sequences. For example “gapped BLAST” may be utilized as described in Altschul et al., 1997, Nucleic Acids Res. 25:3389-3402. Alternatively, PSI-Blast can be used to perform an iterated search which detects distant relationships between molecules. When using any of the above BLAST, Gapped BLAST programs, the default parameters of the respective programs (e.g., XBLAST and NBLAST) may be used.

According to one embodiment, the present invention pertains to a conjugate comprising
(i) the anti-GUCY2C antibody of the invention as disclosed herein,
(ii) at least one toxin, and
(iii) at least one linker connecting said antibody or antibody moiety with said at least one toxin, wherein said antibody specifically binds to human GUCY2C and wherein said at least one toxin is an amatoxin.

Both terms “antibody” and “antibody moiety” as used herein for the conjugates of the invention refer to the anti-GUCY2C antibody or antibodies of the invention as disclosed herein and may be used interchangeably. The term “moiety” may be used to indicate the fact that the anti-GUCY2C antibody of the invention is comprised in the conjugate of the invention.

Accordingly, in one embodiment, the conjugate of the invention comprises the anti-GUCY2C antibody as disclosed herein. Preferably, the anti-GUCY2C antibody of the invention comprises a light chain and a heavy chain according to the table below:

| Light chain (LC) | |
|---|---|
| amino acid sequence | Heavy chain (HC) amino acid sequence |
| SEQ ID NO: 78 | SEQ ID NOs: 80, 81, 82, 83, or 84. |
| SEQ ID NO: 85 | SEQ ID NO: 87, 88, 89, 90, or 91 |
| SEQ ID NO: 92 | SEQ ID NO: 94, 95, 96, 97, or 98 |
| SEQ ID NO: 99 | SEQ ID NO: 101, 102, 103, 104 or 105 |
| SEQ ID NO: 106 | SEQ ID NO: 108, 109, 110, 111, or 112 |
| SEQ ID NO: 113 | SEQ ID NO: 115, 116, 117, 118, or 119 |

More preferably, the conjugate of the invention comprises an anti-GUCY2C antibody moiety which comprises a light chain amino acid sequence and a heavy chain amino acid sequence selected from a light chain amino acid sequence according to SEQ ID NO: 78 and a heavy chain amino acid sequence according to SEQ ID NO: 80, a light chain amino acid sequence according to SEQ ID NO: 85 and a heavy chain amino acid sequence according to SEQ ID NO: 87, a light chain amino acid sequence according to SEQ ID NO: 92 and a heavy chain amino acid sequence according to SEQ ID NO: 94, a light chain amino acid sequence according to SEQ ID NO: 99 and a heavy chain amino acid sequence according to SEQ ID NO: 101, a light chain amino acid sequence according to SEQ ID NO: 106 and a heavy chain amino acid sequence according to SEQ ID NO: 108, and a light chain amino acid sequence according to SEQ ID NO: 113 and a heavy chain amino acid sequence according to SEQ ID NO: 115, more preferably the conjugate of the invention comprises an anti-GUCY2C antibody moiety which comprises a light chain amino acid sequence and a heavy chain amino acid sequence selected fromSEQ ID NO: 78 and SEQ ID NO: 80, or SEQ ID NO: 85 and SEQ ID NO: 87, or SEQ ID NO: 92 and SEQ ID NO: 94.

According to one embodiment the conjugate of the invention comprises at least one, e.g. 1, 2, 3, 4 toxin molecules, whereby the toxin an amatoxin which is bound to the anti-GUCY2C antibody of the invention via at least one, e.g. one, or two linkers.

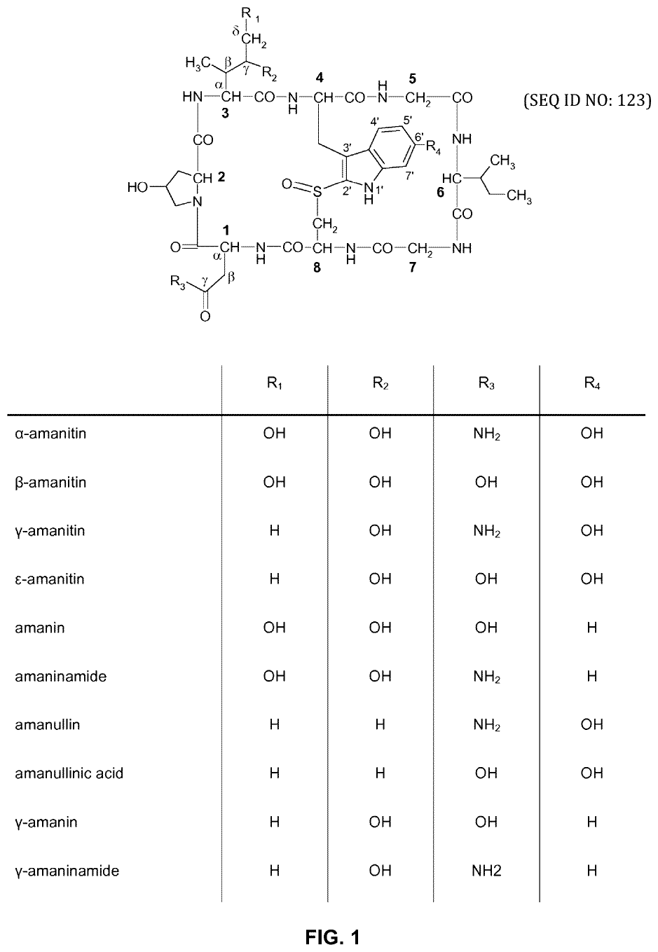

The term “amatoxin” or “amatoxins” as used herein refers to bicyclic peptides composed of 8 amino acids that are found in Amanita phalloides mushrooms (see FIG. 1). Amatoxins specifically inhibit the DNA-dependent RNA polymerase II of mammalian cells, and thereby also the transcription and protein biosynthesis of the affected cells. Inhibition of transcription in a cell causes stop of growth and proliferation. Though not covalently bound, the complex between amanitin and RNA-polymerase II is very tight (K_{D}=3 nM). Dissociation of amanitin from the enzyme is a very slow process, thus making recovery of an affected cell unlikely. When the inhibition of transcription lasts sufficiently long, the cell will undergo programmed cell death (apoptosis).

In the context of the present invention the term “amatoxin” includes all bicyclic peptides composed of 8 amino acids as isolated from the genus Amanita and described in Wieland, T. and Faulstich H. (Wieland T, Faulstich H., CRC Crit Rev Biochem. 5 (1978) 185-260), further all chemical derivatives thereof; further all semisynthetic analogs thereof; further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin.

As used herein, a “derivative” of a compound refers to a species having a chemical structure that is similar to the compound, yet containing at least one chemical group not present in the compound it is derived from and/or deficient of at least one chemical group that is present in the compound it is derived from. The compound to which the derivative is compared to is known as the “parent” compound. Typically, a “derivative” may be produced from the parent compound in one or more chemical reaction steps.

As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%. more preferably at least 40%, and more preferably at least 50%) of the activity of the parent compound. Thus, an “analogue of an amatoxin”, as it is used herein, refers to a compound that is structurally related to any one of α-amanitin, β-amanitin, γ-amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, 50%, 60%, and more preferably at least 70%, 80%, 90%) of the inhibitory activity against mammalian RNA polymerase II as compared to at least one of α-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of α-amanitin, β-amanitin, γ-amanitin, ε-amanitin, amanin, amaninamide, amanullin, or amanullinic acid. The inhibitory activity might be measured by determining the concentration at which 50% inhibition occurs (IC₅₀ value). The inhibitory activity against mammalian RNA polymerase II can be determined indirectly by measuring the inhibitory activity on cell proliferation, or alternatively, the inhibitory activity of the amatoxins and their respective derivatives as disclosed herein may e.g. be assessed using RNA polymerase II activity assasy as disclosed in Voss et al. BMC Molecular Biology 2014, 15:7.

A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes.

According to some embodiments of the present invention, the amatoxin can be selected from the group consisting of α-amanitin, β-amanitin, amanin, amaninamide and analogues, derivatives and salts thereof.

Functionally, amatoxins are defined as peptides or depsipeptides that inhibit mammalian RNA polymerase 11. Preferred amatoxins are those with a functional group (e.g. a carboxylic group, an amino group, a hydroxy group, a thiol or a thiol-capturing group) that can be reacted with linker molecules or target-binding moieties as defined below.

In the context of the present invention, the term “amanitins” particularly refers to bicyclic structures that are based on an aspartic acid or asparagine residue in position 1, a proline residue, particularly a hydroxyproline residue in position 2, an isoleucine, hydroxyisoleucine or dihydroxyisoleucine in position 3 (or aspartic acid for amanullic acid), a tryptophan or hydroxytryptophan residue in position 4 (or proline for proamanullin), glycine residues in positions 5 and 7 (or isoleucine residues in case of amanullic acid and proamanullin), an isoleucine residue in position 6, and a cysteine residue in position 8, particularly a derivative of cysteine that is oxidized to a sulfoxide or sulfone derivative (for the numbering and representative examples of amanitins, see FIG. 1), and furthermore includes all chemical derivatives thereof; further all semisynthetic analogues thereof; further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase 11. Throughout this application the term “amino acid 1” with respect to an amatoxin referes to the asparagine residue of the respective amatoxin as depicted in FIG. 1, accordingly, “amino acid 2” refers to hydroxyproline residue, “amino acid 3” refers to dihydroxyisoleucin residue, “amino acid 4” refers to hydroxytryptophane residue, “amino acid 5” and “amino acid 7” refer to glycine residue, “amino acid 6” refers to isoleucine residue and “amino acid 8” refers to cysteinyl residue.

According to some embodiments of the present invention, said linker is connected to said antibody of the invention via any of the natural Cys residues of said antibody, preferably via a disulfide linkage. Preferred cysteine residues for linking said linker to said anti-GUCY2C antibody of the invention are the cysteine residues which form the interchain disulfide bridges. Such conjugation of said linker to said cysteine residues forming the interchain disulfide bridges of the inventive IgG1 antibody may e.g. be used if said antibody is not a cysteine substituted or cysteine-engineered antibody. Throughout this application the term “cysteine substituted antibody” may be used interchangeably with “cysteine-engineered antibody” both terms referring to antibodies in which at least one naturally occurring amino acid has been mutated to a cysteine as disclosed in e.g. WO2016040856A2, or WO2016142049A1. The use of cysteine-engineered antibodies for the manufacture of the conjugate of the invention is preferred, because the coupling reaction of amatoxin-linker constructs to the anti-GUCY2C antibody of the invention will yield conjugates with a drug-to-antibody ratio (DAR) of about 2. Furthermore, the use of cysteine-engineered antibodies will leave the interchain disulfide bonds of the anti-GUVY2C antibody of the invention intact which results in more stable conjugates compared to those which use interchain disulfide bonds for coupling of the linker-amatoxin constructs. Also, the resulting conjugates of the invention with a DAR of 2 will be characterized by highly similar, or identical pharmacokinetics as a consequence of the near homogenous (e.g. >90%, 95% homogenous) DAR2 distribution of conjugate species compared to interchain conjugates with varying DAR between for example about 1 to about 4. The term “homogenous” as used herein refers to the presence of a single species of antibody-drug conjugate in a given sample, e.g. a near homogenous refers to a sample in which >90%, >95% of the ADC or conjugate species have the same DAR, e.g. a DAR=2.

According to preferred embodiments, said linker is connected via a disulfide linkage between Cys265 of the anti-GUCY2C antibody of the invention and said linker (numbering according to the EU numbering system). Accordingly, the anti-GUCY2C antibody of the invention comprises a genetically engineered Fc region which comprises at least the amino acid substitution D265C (according to EU numbering system), which may also be referred to as Cys265.

According to some embodiments, the linker of the conjugate of the invention is a non-cleavable or a cleavable linker. The term “linker” as used herein means a divalent chemical moiety comprising a covalent bond or a chain of atoms that covalently attaches or covalently links an antibody such as the anti-GUCY2C antibodies of the invention or fragments thereof to an amatoxin as described herein.

A “non-cleavable linker” is understood not to be subject to enzymatical cleavage by e.g. cathepsin B and is released from the conjugates of the invention during degradation (e.g., lysosomal degradation) from the antibody moiety of the conjugate of the invention inside the target cell. Non-cleavable linkers suitable for use according to the invention may e.g. include one or more groups selected from a bond, —(C═O)—, C₁-C₆ alkylene, C₁-C₆ heteroalkylene, Cr Ce alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include (CH₂), (C═O)(CH₂)ₚ, and polyethyleneglycol (PEG; (CH₂CH₂O)ₚ), units, wherein p is an integer from 1-6, independently selected for each occasion.

In some embodiments, the non-cleavable linker according to the invention comprises one or more of a bond, —(C═O)—, a —C(O)NH— group, an —OC(O)NH— group, C1-Ce alkylene, C1-Ce heteroalkylene, C₂-C₆ alkenylene, C₂-C₆ heteroalkenylene, C₂-C₆ alkynylene, C₂-C₆ heteroalkynylene, C₂-C₆ cycloalkylene, heterocycloalkylene, arylene, heteroarylene, a —(CH2CH2O)p-group where p is an integer from 1-6, wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₂-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

For example, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₂-C₆ cycloalkylene, heterocycloalkylene, arylene, or heteroarylene of the non-cleavable linker as disclosed herein may optionally be interrupted by one or more heteroatoms selected from O, S and N and may e.g. be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

According to preferred embodiments, the non-cleavable linker of the conjugate of the invention comprises a —(CH₂)ₙ— unit, where n is an integer from, 2-12, e.g. 4-6, 8, 10, or 2-6, e.g. n is 1, 2, 3, 4, 5, or 6.

In a preferred embodiment, the non-cleavable linker of the conjugate of the invention comprises —(CH₂)ₙ— wherein n is 6 and the linker is represented by the formula:

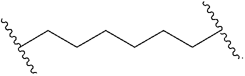 [SMILES: *CCCCCC*]

In some embodiments, the non-cleavable linkers of the invention as disclosed herein further comprise a thiol-reactive group. The thio-reactive group of said non-cleavable linkers as disclosed above may e.g. be selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide.

According to a preferred embodiment, the thiol reactive group is a maleimide (meleimidyl moiety) as disclosed above. For example, the non-cleavable linker comprising said maleimide may e.g. have the following structure, whereby the wavy line at the linker terminus indicates the point of attachment to the amatoxin:

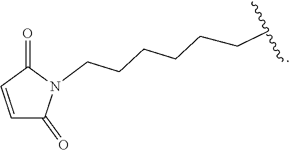 [SMILES: *CCCCCCN1C(=O)C=CC1=O]

Following conjugation to a reactive sulfhydryl on e.g. the anti-GUCY2C antibody as disclosed herein the meleimidyl moiety of e.g. cleavable or non-cleavable linker as disclosed herein comprise the structure:

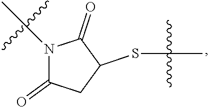 [SMILES: *SC1CC(=O)N(*)C1=O]

whereby the wavy line represents the attachment site of a cleavable or non-cleavable linker as disclosed herein and the sulfur atom is part of a reactive cysteine comprised in the antibody of the invention, preferably Cys265. The above structure may also be referred to as “X” or “Z”.

According to preferred embodiments, the conjugate of the invention comprising a cleavable or non-cleavable linker as disclosed herein and which further comprise a thiol-reactive group may be coupled to a naturally occurring sulfhydryl moiety in the antibody of the conjugate, or said cleavable or non-cleavable linker of the conjugates of the invention comprising a thiol-reactive group may be coupled to a sulfhydryl moiety which has been introduced into the antibody by genetic engineering as described in e.g. Junutula Nat Biotechnol. 2008 August; 26(8):925-32. Preferably, the cleavable or non-cleavable linker as disclosed herein which comprise a thio-reactive group are coupled to sulfhydryl moieties that have been introduced into the Fc region of the anti-GUCY2C antibody of the conjugate of the invention by genetic engineering as disclosed herein above, e.g. D265C (according to EU numbering).

A “cleavable linker” is understood as comprising at least one cleavage site. As used herein, the term “cleavage site” shall refer to a moiety that is susceptible to specific cleavage at a defined position under particular conditions. Said conditions are, e.g., specific enzymes or a reductive environment in specific body or cell compartments.

According to some embodiments, said cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands coordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme's active site. Serine proteases fall into two broad categories based on their structure: chymotrypsin-like (trypsin-like) or subtilisin-like.

Threonine proteases are a family of proteolytic enzymes harboring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In some embodiments of the present invention, the cleavable site is cleavable by at least one agent selected from the group consisting of Cathepsin A or B, matrix metalloproteinases (MMPs), elastases, β-glucuronidase and β-galactosidase, preferably Cathepsin B.

In some embodiments of the present invention, the cleavage site is a disulfide bond and specific cleavage is conducted by a reductive environment, e.g., an intracellular reductive environment, such as, e.g., acidic pH conditions. For example, a corresponding linker may have the following structure:
(amatoxin)-(CH₂)₂—S—S—(CH₂)₂—X—S-(antibody)
(amatoxin)-(CH₂)₃—S—S—(CH₂)₂—X—S-(antibody);
(amatoxin)-(CH₂)₂—S—S—(CH₂)₃—X—S-(antibody);
(amatoxin)-(CH₂)₃—S—S—(CH₂)₃—X—S-(antibody),
   whereby X is as disclosed above.

In some embodiments, the linker is a pH-sensitive linker, and is sensitive to hydrolysis at certain pH values. Typically, the pH-sensitive linker is cleavable under acidic conditions. This cleavage strategy generally takes advantage of the lower pH in the endosomal (pH˜5-6) and lysosomal (pH˜4.8) intracellular compartments, as compared to the cytosol (pH˜7.4), to trigger hydrolysis of an acid labile group in the linker, such as a hydrazone (Jain et al. (2015) Pharm Res 32:3526-40). In some embodiments, the linker is an acid labile and/or hydrolyzable linker. For example, an acid labile linker that is hydrolyzable in the lysosome, and contains an acid labile group (e.g., a hydrazone, a semicarbazone, a thiosemicarbazone, a cis-aconitic amide, an orthoester, an acetal, a ketal, or the like) can be used. See, e.g., U.S. Pat. Nos. 5,122,368; 5,824,805; 5,622,929; Dubowchik and Walker (1999) Pharm. Therapeutics 83:67-123; Neville et al. (1989) Biol. Chem. 264: 14653-61. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond). See, e.g., U.S. Pat. No. 5,622,929.

According to some embodiments of the present invention, the cleavable linker of the invention is a enzymatically cleavable linker. Enzymatically cleavable linker comprise a cleavage site that is an enzymatically cleavable moiety comprising two or more amino acids. Preferably, said enzymatically cleavable moiety comprises a phenylalanine-lysine (Phe-Lys), valine-lysine (Val-Lys), phenylalanine-alanine (Phe-Ala), valine-alanine (Val-Ala), phenylalanine-citrulline (Phe-Cit), or valine-citrulline (Val-Cit) dipeptide, or e.g. a valine-alanine-valine (Val-Ala-Val), leucine-alanine-leucine (Leu-Ala-Leu), glycine-phenylalanine-lysine (Gly-Phe-Lys), isoleucine-alanine-leucine (Ile-Ala-Leu) tripeptide, a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside.

In preferred embodiments, the cleavlable linker of the conjugates of the invention as disclosed above is a self-immolative linker. The term “self-immolative linker” or “self-immolative spacer” refers to a bifunctional chemical moiety that is capable of covalentiy linking two chemical moieties into a normally stable tripartate molecule. The self-immolative spacer is capable of spontaneously separating from the second moiety if the bond to the first moiety is cleaved. Corresponding self-immolative linkers are e.g. disclosed in WO03026577 disclosing p-amidobenzylether-comprising linkers, or in WO2005/112919 and which may e.g. also be used in the conjugates of the invention

In particularly preferred embodiments, the enzymatically cleavable linker according to the invention comprises a dipeptide selected from Phe-Lys, Val-Lys, Phe-Ala, Val-Ala, Phe-Cit and Val-Cit, particularly wherein the cleavable linker further comprises a p-aminobenzyl (PAB) spacer between the dipeptides and the amatoxin:

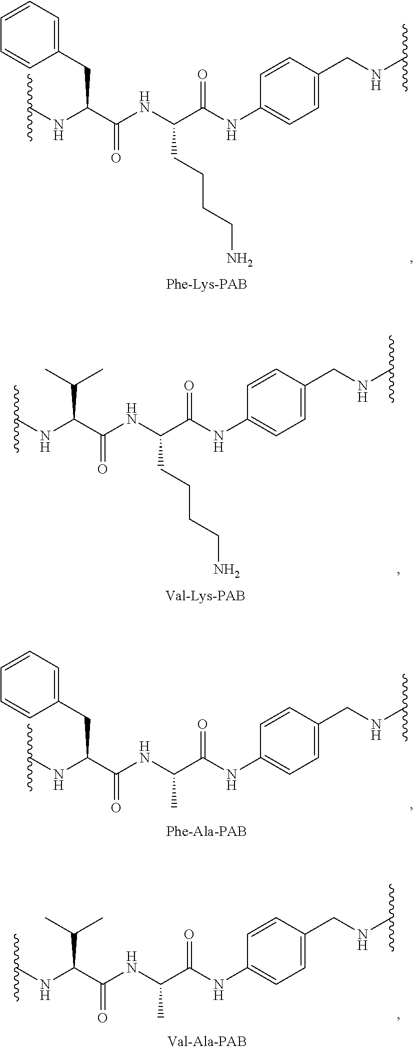 [SMILES: *NCc1ccc(NC(=O)[C@H](CCCCN)NC(=O)[C@H](Cc2ccccc2)N*)cc1 ; *NCc1ccc(NC(=O)[C@H](CCCCN)NC(=O)[C@@H](N*)C(C)C)cc1 ; *NCc1ccc(NC(=O)[C@H](C)NC(=O)[C@H](Cc2ccccc2)N*)cc1 ; *NCc1ccc(NC(=O)[C@H](C)NC(=O)[C@@H](N*)C(C)C)cc1]

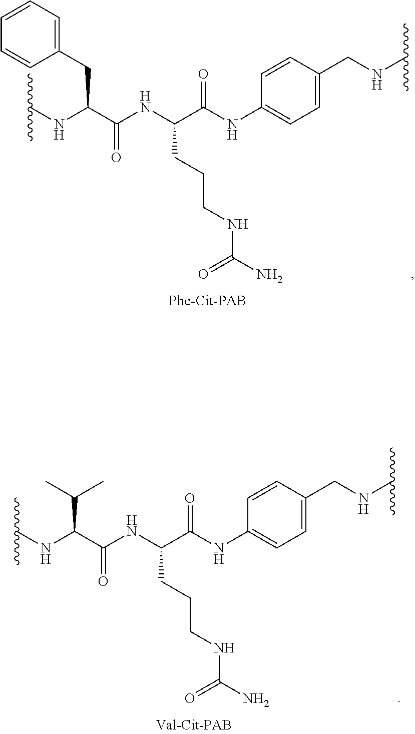 [SMILES: *NCc1ccc(NC(=O)[C@H](CCCNC(N)=O)NC(=O)[C@H](Cc2ccccc2)N*)cc1 ; *NCc1ccc(NC(=O)[C@H](CCCNC(N)=O)NC(=O)[C@@H](N*)C(C)C)cc1]

Accordingly, the conjugates of the invention as disclosed herein can comprise an enzymatically cleavable moiety which comprises any one of the dipeptides-PAB moieties Phe-Lys-PAB, Val-LysPAB, Phe-Ala-PAB, Val-Ala-PAB, Phe-Cit-PAB, or Val-Cit-PAB as disclosed above. Preferably, the cleavable moiety of the conjugates of the invention comprises the dipeptide-PAB moiety Val-Ala-PAB

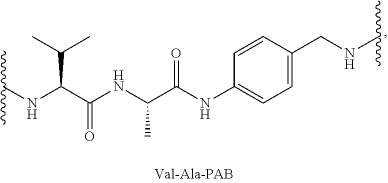 [SMILES: *NCc1ccc(NC(=O)[C@H](C)NC(=O)[C@@H](N*)C(C)C)cc1]

whereby the PAB moiety is linked to the amatoxin.

According to some embodiments, the cleavable moieties or cleavable linkers of the invention as disclosed above comprise a thiol-reactive group, selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide.

According to a preferred embodiment the thiol reactive group is a maleimide (meleimidyl moiety) as depicted below:

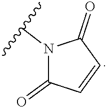 [SMILES: *N1C(=O)C=CC1=O]

Linkers (e.g. cleavable and/or non-cleavable linkers) comprising said thiol-reactive groups are particularly useful for covalent coupling of linker-amatoxin conjugates as disclosed herein to antibodies comprising reactive thiols, such as e.g. cysteine-engineered antibodies comprising at least one reactive cysteine residue for coupling. For example, said linkers are particularly useful for coupling the linker-amatoxin conjugates as disclosed herein to the cysteine-engineered anti-GUCY2C antibody of the invention comprising the amino acid Cys265 (D265C, according to EU numbering system).

According to a particularly preferred embodiment, the linker of the invention comprises the structure (i) prior to coupling, or (ii) following the coupling to an antibody as disclosed herein.

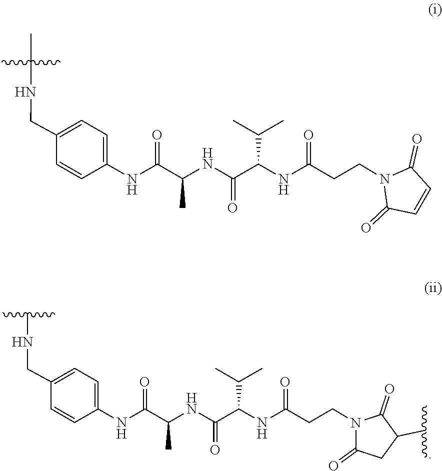 [SMILES: *NCc1ccc(NC(=O)[C@H](C)NC(=O)[C@@H](NC(=O)CCN2C(=O)C=CC2=O)C(C)C)cc1 ; *NCc1ccc(NC(=O)[C@H](C)NC(=O)[C@@H](NC(=O)CCN2C(=O)CC(*)C2=O)C(C)C)cc1]

In some embodiments of the present invention, said conjugate as described comprises an amatoxin comprising (i) an amino acid 4 with a 6′-deoxy position and (ii) an amino acid 8 with an S-deoxy position.

In some embodiments, the linker of the conjugate of the invention is connected to the amatoxin via (i) the γC-atom of amatoxin amino acid 1, or (ii) the 8C-atom of amatoxin amino acid 3, or (iii) the 6′-C-atom of amatoxin amino acid 4. The corresponding attachment sites to which the linker is connected at the amatoxin are indicated in FIG. 1, wherein the attachment site to the γC-atom of amatoxin amino acid 1 is indicated as “R₃”, the attachment site to the δ C-atom of amatoxin amino acid 3 is indicated as “R₁” and the attachment site to the 6′-C-atom of amatoxin amino acid 4 is indicated as “R₄”.

According to preferred embodiments of the present invention, the conjugate of the invention as disclosed herein comprises any of the following compounds of formulas (I) to (XI), respectively, as linker-amatoxin moieties:

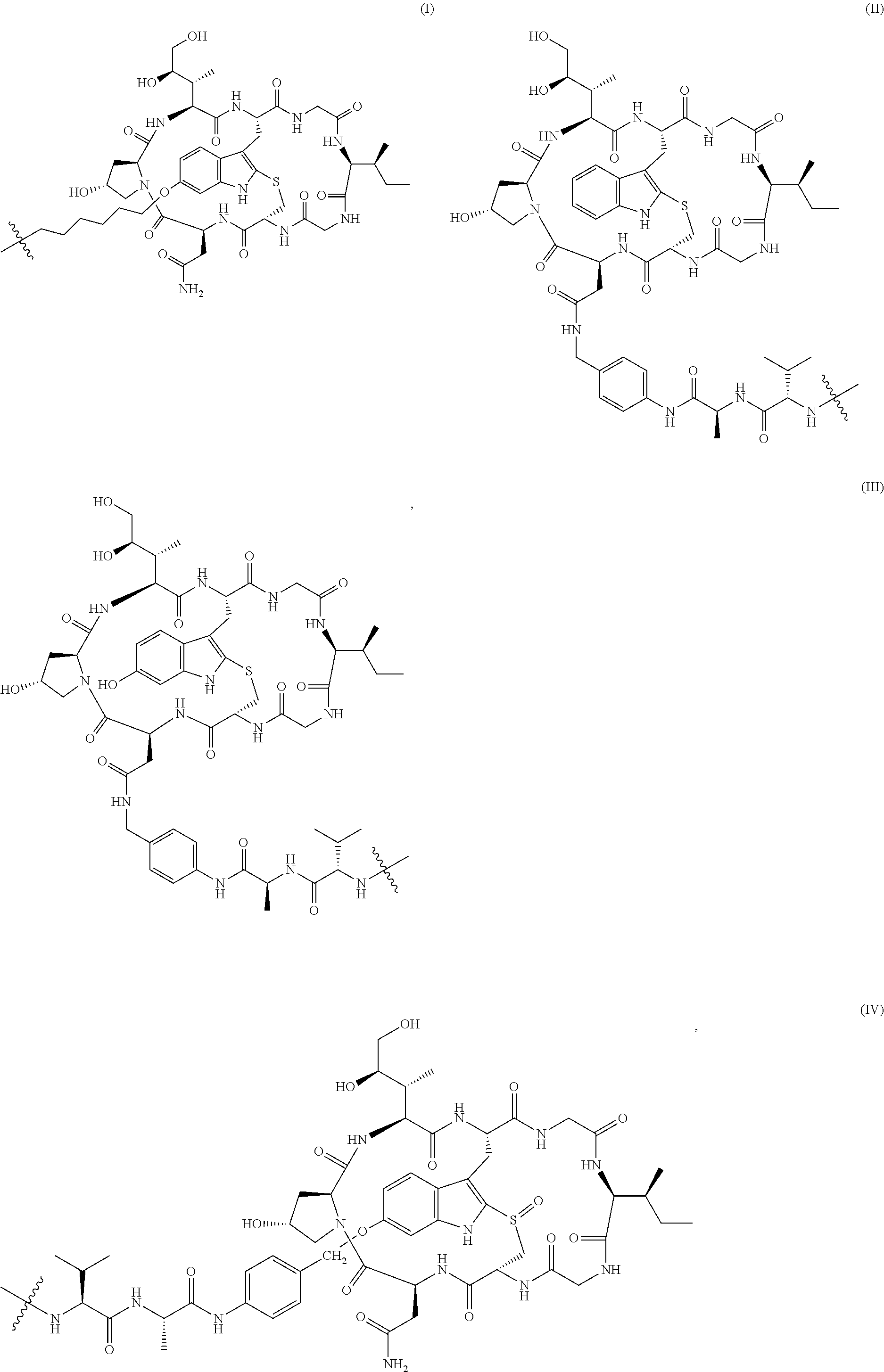

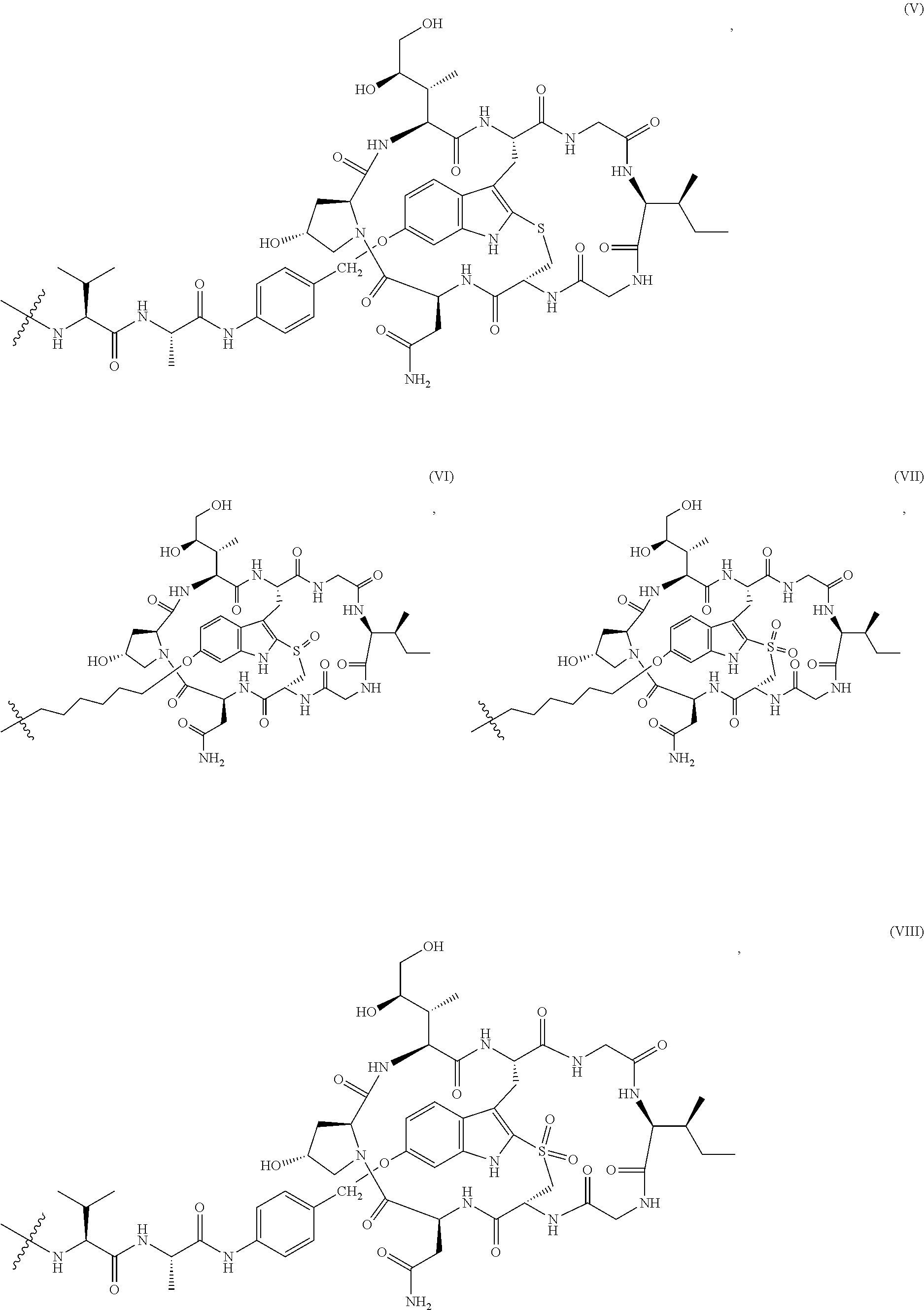

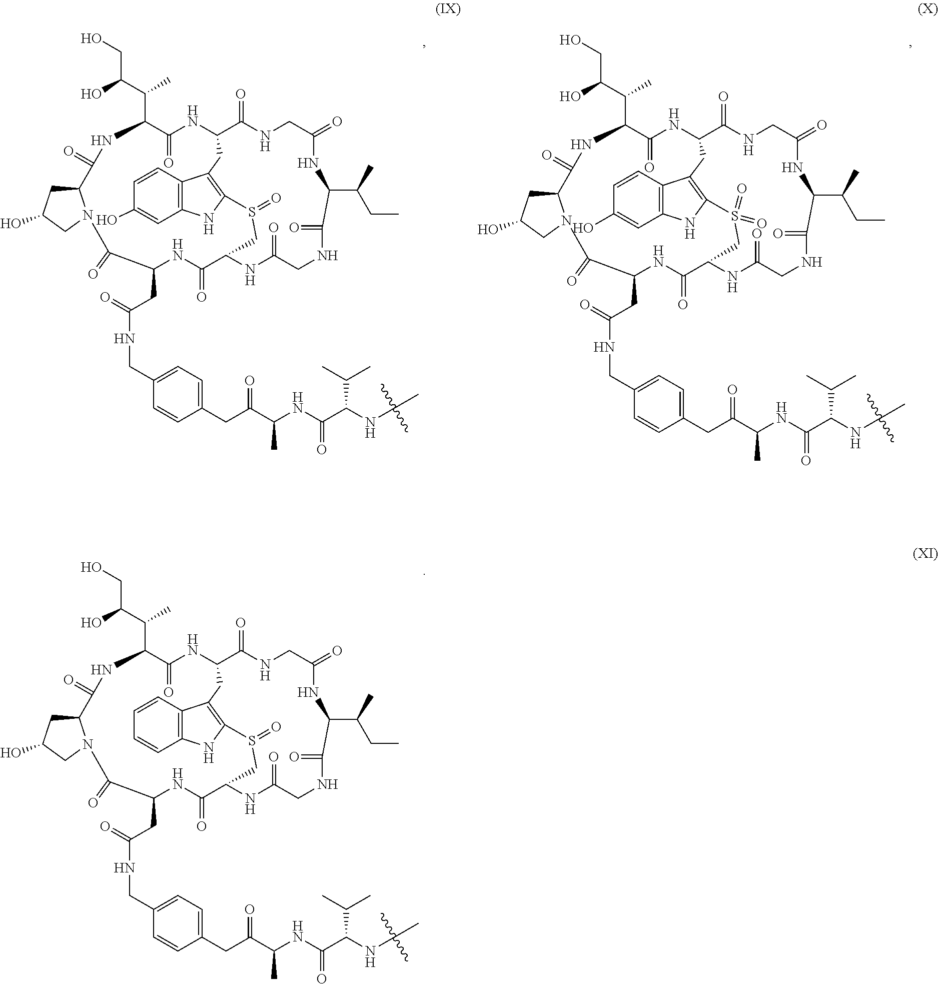

According to preferred embodiments, the conjugate of the invention comprises the anti-GUCY2C antibody as disclosed herein conjugated to amatoxin linker moieties via a thioether linkage according to any one of formula XII to XXII:

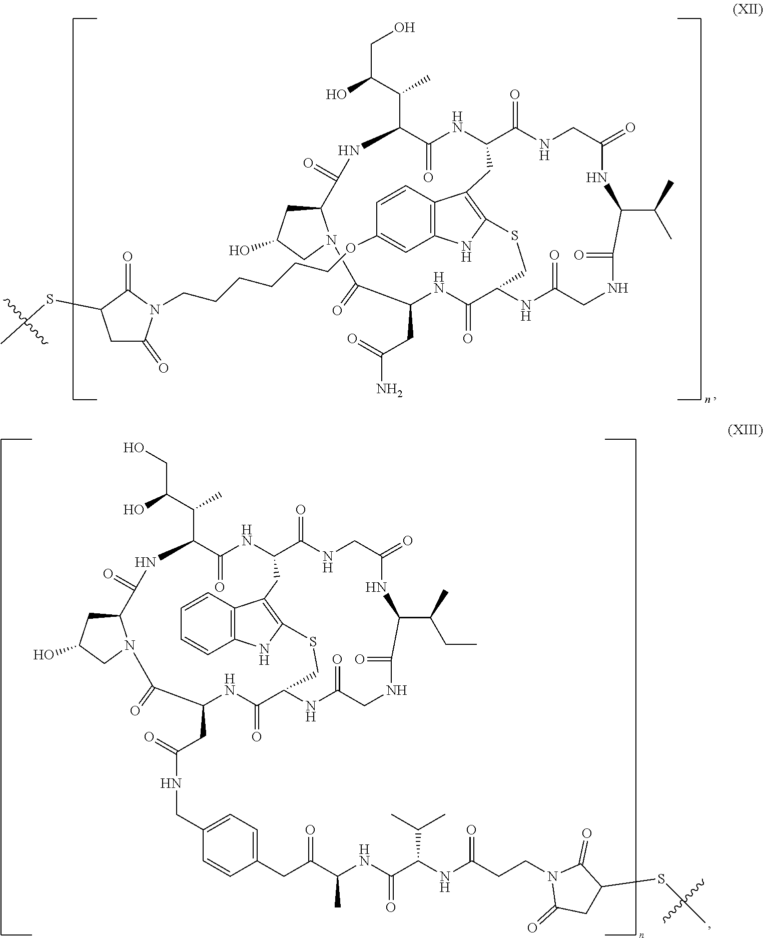

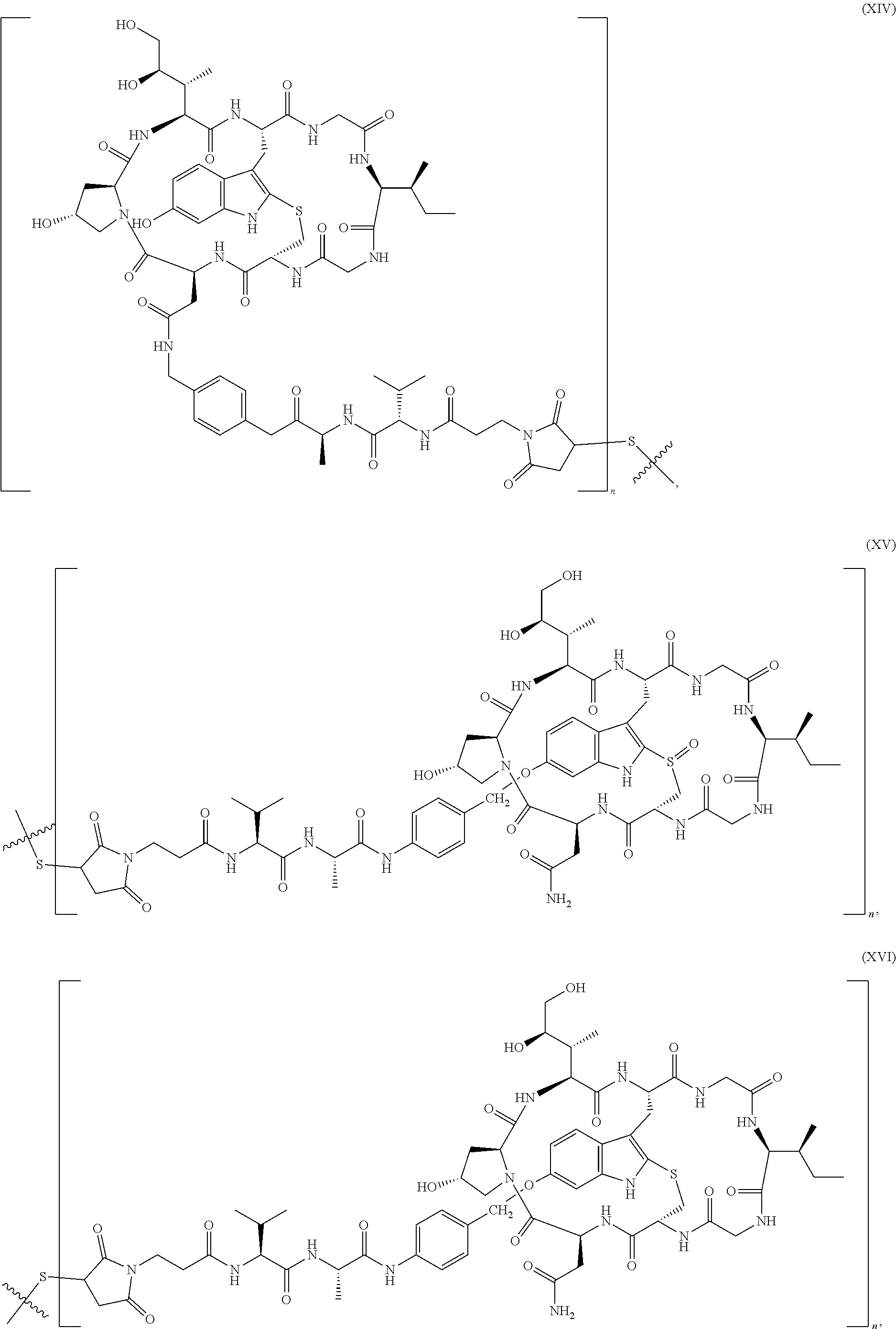

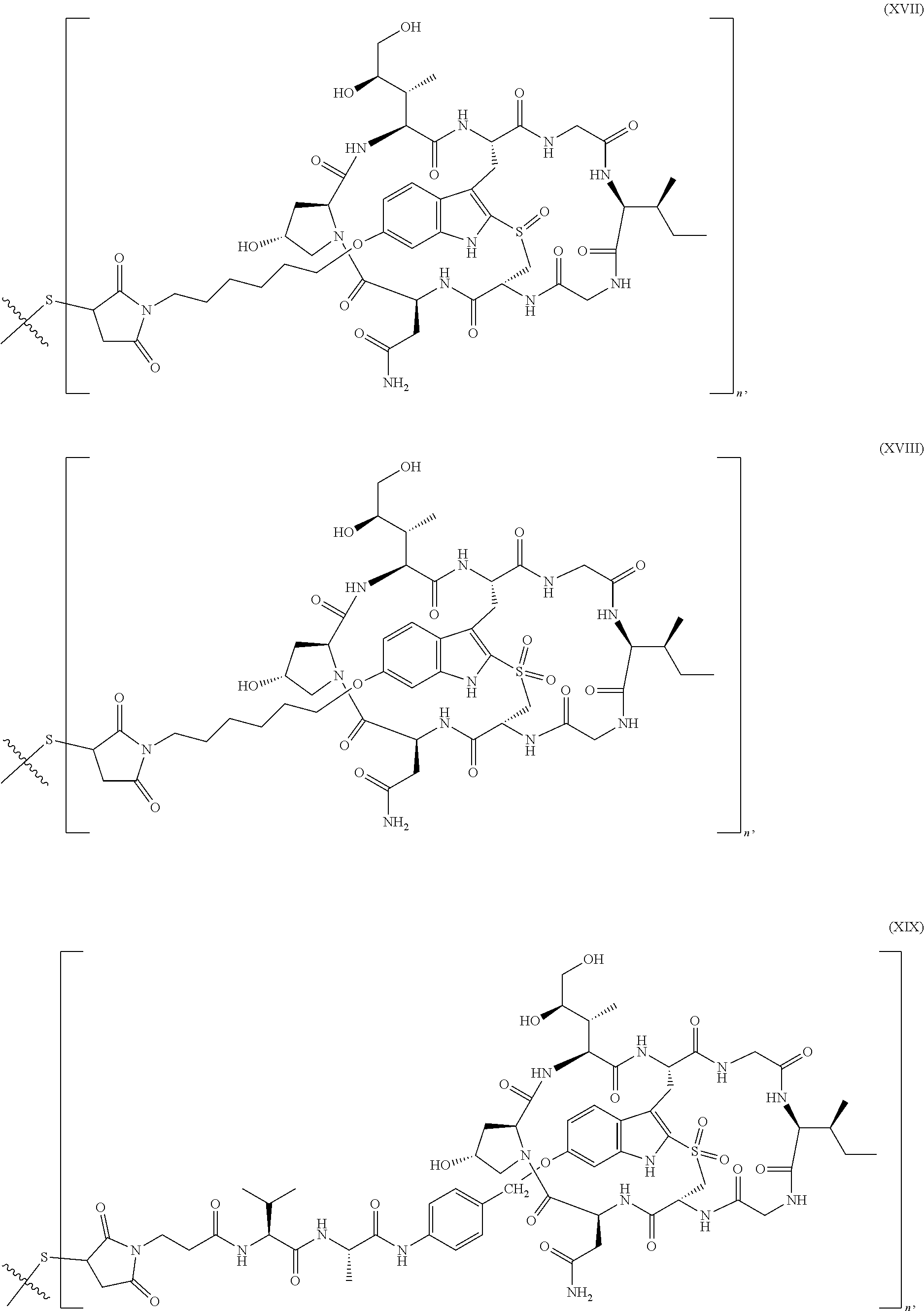

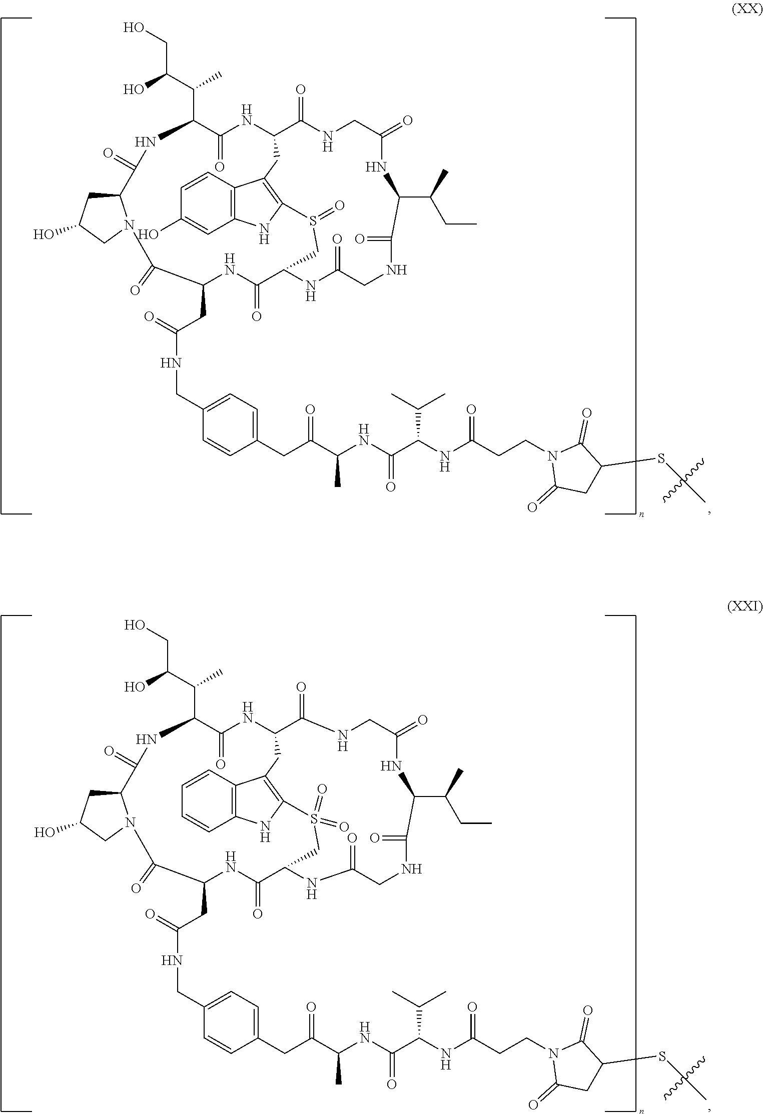

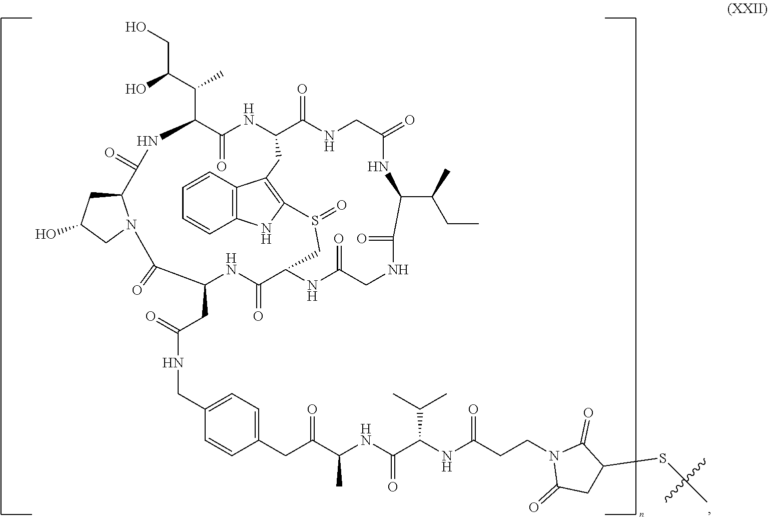

wherein said amatoxin-linker moieties are coupled to the thiol groups of cysteine residues of the anti-GUCY2C antibody of the invention, and wherein n is preferably from about 1, 2, 3, to about 4, 5, 6, 7, 8, preferably, wherein n is from about 1, 1.5, 2, 2.5 to about 3.5, 4.5, 5.5, more preferably, wherein n is from about 1.5 to about 3.5. The thiol groups of cysteine residues of the anti-GUCY2C antibody of the invention may e.g. be naturally occurring cysteine residues of the antibody such as those forming the interchain disulfide bonds following reduction of said cysteine residues for thiol-based conjugation, or said thiol groups of cysteine residues of the anti-GUCY2C antibody of the invention may be genetically engineered, preferably at position D265 (according to EU numbering, D265C).

According to preferred embodiments, the number n of amatoxin-linker conjugates covalently bound to the anti-GUCY2C antibody of the invention is from about 1 to about 2, 3, 4, 6, 7, 8, or from about 1, 2 to about 3, 4, or from about 1.5, 1.6, 1.7, 1.8 to about 1.9, 2.0, 2.1, 2.2, 2.5, 3 or from about 1.9, 2.0 to about 2.1, 2.2, preferably n is about 2 (e.g. 1.8 to about 2.2).

According to preferred embodiments, the conjugate of the invention is selected from the group consisting of
a conjugate (XXIII) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XII) via thioether linkage of the linker with the sulfhydryl group of heavy chain 265Cys residue according to the EU numbering system of said antibody,
a conjugate (XXIV) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (X111) via thioether linkage of the linker with the sulfhydryl group of heavy chain 265Cys residue according to the EU numbering system of said antibody,
a conjugate (XXV) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XIV) via thioether linkage of the linker with the sulfhydryl group of heavy chain 265Cys residue according to the EU numbering system of said antibody,
a conjugate (XXVI) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XV) via thioether linkage of the linker with the sulfhydryl group of heavy chain 265Cys residue according to the EU numbering system of said antibody,
a conjugate (XXVII) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XVI) via thioether linkage of the linker with the sulfhydryl group of heavy chain 265Cys residue according to the EU numbering system of said antibody,
a conjugate (XXVIII) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XVII) via thioether linkage of the linker with the sulfhydryl group of heavy chain 265Cys residue according to the EU numbering system of said antibody,
a conjugate (XXIX) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XVIII) via thioether linkage of the linker with the sulfhydryl group of heavy chain 265Cys residue according to the EU numbering system of said antibody,
a conjugate (XXX) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XIX) via thioether linkage of the linker with the sulfhydryl group of heavy chain 265Cys residue according to the EU numbering system of said antibody,
a conjugate (XXXI) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XX) via thioether linkage of the linker with the sulfhydryl group of heavy chain 265Cys residue according to the EU numbering system of said antibody,
a conjugate (XXXII) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XXI) via thioether linkage of the linker with the sulfhydryl group of heavy chain 265Cys residue according to the EU numbering system of said antibody,
a conjugate (XXXIII) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XXII) via thioether linkage of the linker with the sulfhydryl group of heavy chain 265Cys residue according to the EU numbering system of said antibody, wherein n is from about 1 to about 2.

According to preferred embodiments, the conjugate of the invention is selected from the group consisting of
a conjugate (XXXIV) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XII) via thioether linkage of the linker with the sulfhydryl group of heavy chain 265Cys residue according to the EU numbering system of said antibody,
a conjugate (XXXV) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XIII) via thioether linkage of the linker with the sulfhydryl group of heavy chain 265Cys residue according to the EU numbering system of said antibody,
a conjugate (XXXVI) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XIV) via thioether linkage of the linker with the sulfhydryl group of heavy chain 265Cys residue according to the EU numbering system of said antibody,
a conjugate (XXXVII) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XV) via thioether linkage of the linker with the sulfhydryl group of heavy chain 265Cys residue according to the EU numbering system of said antibody,
a conjugate (XXXVIII) comprising an antibody consisting of two heavy chains, each heavy chain comprising a VH domain consisting or comprising an amino acid sequence according to SEQ ID No. 62, and two light chains, each light chain comprising a VL domain consisting or comprising an amino acid sequence according to SEQ ID No. 56, conjugated to at least one amatoxin-linker moiety of formula (XVI) via thioether linkage of the linker with the sulfhydryl group of heavy chain 265Cys residue according to the EU numbering system of said antibody,
a conjugate (XXXIX) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XVII) via thioether linkage of the linker with the sulfhydryl group of heavy chain 265Cys residue according to the EU numbering system of said antibody,
a conjugate (XL) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XVIII) via thioether linkage of the linker with the sulfhydryl group of heavy chain 265Cys residue according to the EU numbering system of said antibody,
a conjugate (XLI) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XIX) via thioether linkage of the linker with the sulfhydryl group of heavy chain 265Cys residue according to the EU numbering system of said antibody,
a conjugate (XLII) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XX) via thioether linkage of the linker with the sulfhydryl group of heavy chain 265Cys residue according to the EU numbering system of said antibody,
a conjugate (XLIII) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XXI) via thioether linkage of the linker with the sulfhydryl group of heavy chain 265Cys residue according to the EU numbering system of said antibody,
a conjugate (XLIV) comprising an antibody consisting of two heavy chains, each heavy chain comprising a VH domain consisting or comprising an amino acid sequence according to SEQ ID No. 63, and two light chains, each light chain comprising a VL domain consisting or comprising an amino acid sequence according to SEQ ID No. 57, conjugated to at least one amatoxin-linker moiety of formula (XXII) via thioether linkage of the linker with the sulfhydryl group of heavy chain 265Cys residue according to the EU numbering system of said antibody, wherein n is from about 1 to about 2.

According to preferred embodiments, the conjugate of the invention is selected from the group consisting of
a conjugate (XLV) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XII) via thioether linkage of the linker with the sulfhydryl group of heavy chain 265Cys residue according to the EU numbering system of said antibody,
a conjugate (XLVI) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XIII) via thioether linkage of the linker with the sulfhydryl group of heavy chain 265Cys residue according to the EU numbering system of said antibody,
a conjugate (XLVII) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XIV) via thioether linkage of the linker with the sulfhydryl group of heavy chain 265Cys residue according to the EU numbering system of said antibody,
a conjugate (XLVIII) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XV) via thioether linkage of the linker with the sulfhydryl group of heavy chain 265Cys residue according to the EU numbering system of said antibody,
a conjugate (XLIX) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XVI) via thioether linkage of the linker with the sulfhydryl group of heavy chain 265Cys residue according to the EU numbering system of said antibody,
a conjugate (L) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XVII) via thioether linkage of the linker with the sulfhydryl group of heavy chain 265Cys residue according to the EU numbering system of said antibody,
a conjugate (LI) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XVIII) via thioether linkage of the linker with the sulfhydryl group of heavy chain 265Cys residue according to the EU numbering system of said antibody,
a conjugate (LII) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XIX) via thioether linkage of the linker with the sulfhydryl group of heavy chain 265Cys residue according to the EU numbering system of said antibody,
a conjugate (LIII) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XX) via thioether linkage of the linker with the sulfhydryl group of heavy chain 265Cys residue according to the EU numbering system of said antibody,
a conjugate (LIV) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XXI) via thioether linkage of the linker with the sulfhydryl group of heavy chain 265Cys residue according to the EU numbering system of said antibody,
a conjugate (LV) comprising an antibody consisting of two heavy chains, each heavy chain comprising a VH domain consisting or comprising an amino acid sequence according to SEQ ID No. 64, and two light chains, each light chain comprising a VL domain consisting or comprising an amino acid sequence according to SEQ ID No. 58, conjugated to at least one amatoxin-linker moiety of formula (XXII) via thioether linkage of the linker with the sulfhydryl group of heavy chain 265Cys residue according to the EU numbering system of said antibody, wherein n is from about 1 to about 2.

According to preferred embodiments, the conjugate of the invention is selected from the group consisting of
a conjugate (LVI) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XII) via thioether linkage of the linker with the sulfhydryl group of heavy chain 265Cys residue according to the EU numbering system of said antibody,
a conjugate (LVII) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XIII) via thioether linkage of the linker with the sulfhydryl group of heavy chain 265Cys residue according to the EU numbering system of said antibody,
a conjugate (LVIII) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XIV) via thioether linkage of the linker with the sulfhydryl group of heavy chain 265Cys residue according to the EU numbering system of said antibody,
a conjugate (LIX) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XV) via thioether linkage of the linker with the sulfhydryl group of heavy chain 265Cys residue according to the EU numbering system of said antibody,
a conjugate (LX) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XVI) via thioether linkage of the linker with the sulfhydryl group of heavy chain 265Cys residue according to the EU numbering system of said antibody,
a conjugate (LXI) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XVII) via thioether linkage of the linker with the sulfhydryl group of heavy chain 265Cys residue according to the EU numbering system of said antibody,
a conjugate (LXII) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XVIII) via thioether linkage of the linker with the sulfhydryl group of heavy chain 265Cys residue according to the EU numbering system of said antibody,
a conjugate (LXIII) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XIX) via thioether linkage of the linker with the sulfhydryl group of heavy chain 265Cys residue according to the EU numbering system of said antibody,
a conjugate (LXIV) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XX) via thioether linkage of the linker with the sulfhydryl group of heavy chain 265Cys residue according to the EU numbering system of said antibody,
a conjugate (LXV) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XXI) via thioether linkage of the linker with the sulfhydryl group of heavy chain 265Cys residue according to the EU numbering system of said antibody,
a conjugate (LXVI) comprising an antibody consisting of two heavy chains, each heavy chain comprising a VH domain consisting or comprising an amino acid sequence according to SEQ ID No. 65, and two light chains, each light chain comprising a VL domain consisting or comprising an amino acid sequence according to SEQ ID No. 59, conjugated to at least one amatoxin-linker moiety of formula (XXII) via thioether linkage of the linker with the sulfhydryl group of heavy chain 265Cys residue according to the EU numbering system of said antibody, wherein n is from about 1 to about 2.

According to preferred embodiments, the conjugate of the invention is selected from the group consisting of
a conjugate (LXVII) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XII) via thioether linkage of the linker with the sulfhydryl group of heavy chain 265Cys residue according to the EU numbering system of said antibody,
a conjugate (LXVIII) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XIII) via thioether linkage of the linker with the sulfhydryl group of heavy chain 265Cys residue according to the EU numbering system of said antibody,
a conjugate (LXIX) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XIV) via thioether linkage of the linker with the sulfhydryl group of heavy chain 265Cys residue according to the EU numbering system of said antibody,
a conjugate (LXX) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XV) via thioether linkage of the linker with the sulfhydryl group of heavy chain 265Cys residue according to the EU numbering system of said antibody,
a conjugate (LXXI) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XVI) via thioether linkage of the linker with the sulfhydryl group of heavy chain 265Cys residue according to the EU numbering system of said antibody,
a conjugate (LXXII) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XVII) via thioether linkage of the linker with the sulfhydryl group of heavy chain 265Cys residue according to the EU numbering system of said antibody,
a conjugate (LXXIII) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XVIII) via thioether linkage of the linker with the sulfhydryl group of heavy chain 265Cys residue according to the EU numbering system of said antibody,
a conjugate (LXXIV) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XIX) via thioether linkage of the linker with the sulfhydryl group of heavy chain 265Cys residue according to the EU numbering system of said antibody,
a conjugate (LXXV) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XX) via thioether linkage of the linker with the sulfhydryl group of heavy chain 265Cys residue according to the EU numbering system of said antibody,
a conjugate (LXXVI) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XXI) via thioether linkage of the linker with the sulfhydryl group of heavy chain 265Cys residue according to the EU numbering system of said antibody,
a conjugate (LXXVII) comprising an antibody consisting of two heavy chains, each heavy chain comprising a VH domain consisting or comprising an amino acid sequence according to SEQ ID No. 66, and two light chains, each light chain comprising a VL domain consisting or comprising an amino acid sequence according to SEQ ID No. 60, conjugated to at least one amatoxin-linker moiety of formula (XXII) via thioether linkage of the linker with the sulfhydryl group of heavy chain 265Cys residue according to the EU numbering system of said antibody,
wherein n is from about 1 to about 2.

According to preferred embodiments, the conjugate of the invention is selected from the group consisting of
a conjugate (LXXVIII) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XII) via thioether linkage of the linker with the sulfhydryl group of heavy chain 265Cys residue according to the EU numbering system of said antibody,
a conjugate (LXXIX) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XIII) via thioether linkage of the linker with the sulfhydryl group of heavy chain 265Cys residue according to the EU numbering system of said antibody,
a conjugate (LXXX) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XIV) via thioether linkage of the linker with the sulfhydryl group of heavy chain 265Cys residue according to the EU numbering system of said antibody,
a conjugate (LXXXI) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XV) via thioether linkage of the linker with the sulfhydryl group of heavy chain 265Cys residue according to the EU numbering system of said antibody,
a conjugate (LXXXII) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XVI) via thioether linkage of the linker with the sulfhydryl group of heavy chain 265Cys residue according to the EU numbering system of said antibody,
a conjugate (LXXXIII) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XVII) via thioether linkage of the linker with the sulfhydryl group of heavy chain 265Cys residue according to the EU numbering system of said antibody,
a conjugate (LXXXIV) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XVIII) via thioether linkage of the linker with the sulfhydryl group of heavy chain 265Cys residue according to the EU numbering system of said antibody,
a conjugate (LXXXV) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XIX) via thioether linkage of the linker with the sulfhydryl group of heavy chain 265Cys residue according to the EU numbering system of said antibody,
a conjugate (LXXXVI) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XX) via thioether linkage of the linker with the sulfhydryl group of heavy chain 265Cys residue according to the EU numbering system of said antibody,
a conjugate (LXXXVII) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XXI) via thioether linkage of the linker with the sulfhydryl group of heavy chain 265Cys residue according to the EU numbering system of said antibody,
a conjugate (LXXXVIII) comprising an antibody consisting of two heavy chains, each heavy chain comprising a VH domain consisting or comprising an amino acid sequence according to SEQ ID No. 67, and two light chains, each light chain comprising a VL domain consisting or comprising an amino acid sequence according to SEQ ID No. 61, conjugated to at least one amatoxin-linker moiety of formula (XXII) via thioether linkage of the linker with the sulfhydryl group of heavy chain 265Cys residue according to the EU numbering system of said antibody,
wherein n is from about 1 to about 2.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 34 and two light chains, each light chain consisting or comprising an amino acid sequence according to SEQ ID No. 78, conjugated to at least one amatoxin-linker moiety of formula (XIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XIV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XVI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XVII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XVIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XIX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XXI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 79, 80, 81, 82, 83, 84 and two light chains, each light chain consisting or comprising an amino acid sequence according to SEQ ID No. 78, conjugated to at least one amatoxin-linker moiety of formula (XXII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XIV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XVI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XVII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XVIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XIX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XXI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 86, 87, 88, 89, 90, or 91 and two light chains, each light chain consisting or comprising an amino acid sequence according to SEQ ID No. 85, conjugated to at least one amatoxin-linker moiety of formula (XXII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XIV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XVI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XVII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XVIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XIX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XXI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No 93, 94, 95, 96, 97, or 98 and two light chains, each light chain consisting or comprising an amino acid sequence according to SEQ ID No. 92, conjugated to at least one amatoxin-linker moiety of formula (XXII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XIV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XVI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XVII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XVIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XIX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XXI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XXII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 100, 101, 102, 103, 104, or 105 and two light chains, each light chain consisting or comprising an amino acid sequence according to SEQ ID No. 99, conjugated to at least one amatoxin-linker moiety of formula (XXII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 107, 108, 109, 110, 111, or 112 and two light chains, each light chain consisting or comprising an amino acid sequence according to SEQ ID No. 106, conjugated to at least one amatoxin-linker moiety of formula (XII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XIV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XVI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XVII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XVIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XIX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XXI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 107, 108, 109, 110, 111, or 112 and two light chains, each light chain consisting or comprising an amino acid sequence according to SEQ ID No. 106, conjugated to at least one amatoxin-linker moiety of formula (XXII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No. 114, 115, 116, 117, 118, or 119 and two light chains, each light chain consisting or comprising an amino acid sequence according to SEQ ID No. 113, conjugated to at least one amatoxin-linker moiety of formula (XII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XIV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XV) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XVI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XVII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XVIII) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XIX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XX) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XXI) via thioether linkage of the linker with the sulfhydryl group of heavy chain 265Cys residue according to the EU numbering system of said antibody.

According to some embodiments, the conjugate of the invention comprises an anti-GUCY2C antibody consisting of two heavy chains, each heavy chain consisting of or comprising an amino acid sequence according to one of SEQ ID No., 114, 115, 116, 117, 118, or 119 and two light chains, each light chain consisting or comprising an amino acid sequence according to SEQ ID No. 113, conjugated to at least one amatoxin-linker moiety of formula (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain comprising an amino acid sequence according to of SEQ ID No. 62 and two light chains, each light chain comprising an amino acid sequence according to SEQ ID No. 56, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain comprising an amino acid sequence according to of SEQ ID No. 63 and two light chains, each light chain comprising an amino acid sequence according to SEQ ID No. 57, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain consisting of or comprising an amino acid sequence according to of SEQ ID No. 80 and two light chains, each light chain consisting or comprising an amino acid sequence according to SEQ ID No. 78, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain consisting of or comprising an amino acid sequence according to of SEQ ID No. 87 and two light chains, each light chain consisting or comprising an amino acid sequence according to SEQ ID No. 85, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain consisting of or comprising an amino acid sequence according to of SEQ ID No. 94 and two light chains, each light chain consisting or comprising an amino acid sequence according to SEQ ID No. 92, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain consisting of or comprising an amino acid sequence according to of SEQ ID No. 101 and two light chains, each light chain consisting or comprising an amino acid sequence according to SEQ ID No. 99, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain consisting of or comprising an amino acid sequence according to of SEQ ID No. 108 and two light chains, each light chain consisting or comprising an amino acid sequence according to SEQ ID No. 106, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to more preferred embodiments, the conjugate of the invention comprises an anti-GUCY2C antibody consisting of two heavy chains, each heavy chain consisting of or comprising an amino acid sequence according to of SEQ ID No. 115 and two light chains, each light chain consisting or comprising an amino acid sequence according to SEQ ID No. 113, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to one particularly preferred embodiment, the conjugate of the invention comprises an anti-GUCY2C antibody consisting of two heavy chains, each heavy chain consisting of or comprising an amino acid sequence according to of SEQ ID No. 80 and two light chains, each light chain consisting or comprising an amino acid sequence according to SEQ ID No. 78, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody.

According to one particularly preferred embodiment, the conjugate of the invention comprises an anti-GUCY2C antibody consisting of two heavy chains, each heavy chain consisting of or comprising an amino acid sequence according to of SEQ ID No. 87 and two light chains, each light chain consisting or comprising an amino acid sequence according to SEQ ID No. 85, conjugated to at least one amatoxin-linker moiety selected from formula (XII), (XIII), (XIV), (XV), (XVI), (XVII), (XVIII), (XIX), (XX), (XXI), (XXII) via thioether linkage of the linker with the sulfhydryl group of heavy chain 265Cys residue according to the EU numbering system of said antibody, even more preferably the amatoxin-linker moiety is selected from one of formulae (XII), (XIII), or (XIV).

In some embodiments, the conjugate according to the present invention as disclosed above, can have a cytotoxic activity of an IC₅₀ better than 10×10⁻¹⁰ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, preferably better than 10×10⁻¹⁰ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, and more preferably better than 9×10⁻¹¹ M, 8×10⁻¹¹ M, 7×10⁻¹¹ M, 6×10⁻¹¹ M, 5×10⁻¹¹ M, 4×10⁻¹¹ M, 3×10⁻¹¹ M, 2×10⁻¹¹ M, or 1×10⁻¹¹ M.

In one embodiment, the invention provides a polynucleotide encoding an amino acid sequence comprising any of SEQ ID NO: 62, 63, 64, 65, 66, or 67.

According to one embodiment, the invention provides a polynucleotide encoding an amino acid sequence comprising any of SEQ ID NO: 56, 57, 58, 59, 60, or 61.

According to one embodiment, the polynucleotide of the invention encodes an amino acid sequence comprising the amino acid sequences selected from the following combinations of V_{H} and V_{L} sequences:
SEQ ID NOs: 62, 56
SEQ ID NOs: 63, 57
SEQ ID Nos: 64, 58
SEQ ID Nos: 65, 59
SEQ ID Nos: 66, 60
SEQ ID NO: 67, 61,
   or amino acid sequences that are at least 90%, 95% similar to any of the above V_{L} and V_{H} sequences, singly or in combination as disclosed above.

The term “polynucleotide” as used herein relates to a nucleic acid sequence. The nucleic acid sequence may be a DNA or a RNA sequence, preferably the nucleic acid sequence is a DNA sequence. The polynucleotides of the present invention shall be provided, preferably, either as an isolated polynucleotide (i.e. isolated from its natural context) or in genetically modified form. An isolated polynucleotide as referred to herein may e.g. also encompass polynucleotides which are present in cellular context other than their natural cellular context, i.e. heterologous polynucleotides. The term polynucleotides encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides. According to one embodiment, the present invention provides an expression vector comprising the polynucleotides of the invention. Expression vectors according to the invention may e.g. comprise at least one polynucleotide encoding an amino acid sequence comprising an amino acid sequence selected from SEQ ID NO: 62, 63, 64, 65, 66, or 67 or at least one polynucleotide encoding an amino acid sequence comprising an amino acid sequence selected from SEQ ID NO: 56, 57, 58, 59, 60, or 61. The expression vector of the invention may e.g. also comprise one polynucleotide encoding an amino acid sequence comprising an amino acid sequence selected from SEQ ID NO: 62, 63, 64, 65, 66, or 67, and one polynucleotide encoding an amino acid sequence comprising an amino acid sequence selected from NO: 56, 57, 58, 59, 60, or 61, e.g. the expression vector may comprise polynucleotides encoding an amino acid sequence comprising an amino acid sequence according to SEQ ID NO: 62, 56; SEQ ID NO: 63, 57; SEQ ID NO: 64, 58; SEQ ID NO: 65, 59; SEQ ID NO: 66, 60; or SEQ ID NO: 67, 61. The term “expression vector” or “vector”, preferably, encompasses plasmid, phage, viral or retroviral vectors as well artificial chromosomes, such as bacterial or yeast artificial chromosomes. The expression vector comprising the polynucleotides of the present invention, preferably, further comprises selectable markers for propagation and/or selection in a suitable host cell. The polynucleotides according to the invention comprised in the expression vector are operatively linked to expression control sequences allowing expression or propagation in prokaryotic or eukaryotic cells, particularly in eukaryotic cells or isolated fractions thereof. Expression of said polynucleotide comprises transcription of the polynucleotide, preferably into a translatable mRNA. Expression vectors of the invention furthermore comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. Expression vectors are known in the art and include e.g. pBluescript (Stratagene), pCDM8, pRc/CMV, pcDNA1, pcDNA3 (Invitrogene) or pSPORT1 (GIBCO BRL). The expression vectors of the invention may e.g. be incorporated into a host cell by various techniques known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the expression vector be a virus, it may be packaged in vitro using an appropriate packaging cell line prior to application to host cells. Retroviral vectors may be replication competent or replication defective. In the latter case, viral propagation generally will occur only in complementing host/cells. Available transformation and transfection techniques are reviewed in Fus-Kujawa A. et al (2021) An Overview of Methods and Tools for Transfection of Eukaryotic Cells in vitro. Front. Bioeng. Biotechnol. 9:701031, or in Chong Z X, et al. (2021) Transfection types, methods and strategies: a technical review. PeerJ 9:e11165, transfection protocols are e.g. described in “Molecular Cloning—A laboratory manual”, 4^{th} edition (2001), ISBN 978-1-936113-42-2, chapter 15, pages 1131-1203.

According to one embodiment, the present invention pertains to a host cell comprising the expression vector of the invention, or a polynucleotide of the invention as disclosed herein. The term “host cell” as used herein refers to a prokaryotic or eukaryotic cell that comprises the expression vector or polynucleotide of the invention. It is preferred that the host cell of the invention is eukarytic cell, such as a yeast cell (e.g. Saccharomyces cerevisiae, Hansenula polymorpha, Schizosaccharomyces pombe, Schwanniomyces occidentalis, Kluyveromryceslacts, Yarrowia lipolytica and Pichia pastoris), insect cell (e.g. Sf9, Sf21, S2, Hi5, or BTI-TN-5B1-4), more preferably, the host cell of the invention is a mammalian cell selected from HEK293, HEK293T, HEK293E, HEK 293F, NS0, per.C6, MCF-7, HeLa, Cos-1, Cos-7, PC-12, 3T3, Vero, vero-76, PC3, U87, SAOS-2, LNCAP, DU145, A431, A549, B35, H1299, HUVEC, Jurkat, MDA-MB-231, MDA-MB-468, MDA-MB-435, Caco-2, CHO, CHO-K1, CHO-B11, CHO-DG44, BHK, AGE1.HN, Namalwa, WI-38, MRC-5, HepG2, L-929, RAB-9, SIRC, RK13, 11B11, 1D3, 2.4G2, A-10, B-35, C-6, F4/80, IEC-18, L2, MH1C1, NRK, NRK-49F, NRK-52E, RMC, CV-1, BT, MDBK, CPAE, MDCK.1, MDCK.2, and D-17, more preferably the host cell is selected from CHO, CHO-K1, CHO-B11, CHO-DG44, HEK293, HEK293T, HEK293E, HEK 293F, NS0, or per.C6 cells.

In one embodiment, the present invention pertains to the use of the anti-GUCY2C antibody of the invention in the manufacture of a conjugate of the invention as disclosed herein.

According to a second aspect, it is an object of the invention to provide a pharmaceutical composition which comprises the conjugate of the invention as disclosed herein.

Said pharmaceutical composition further may comprise e.g. one or more pharmaceutically acceptable buffers, surfactants, diluents, carriers, excipients, fillers, binders, lubricants, glidants, disintegrants, adsorbents, and/or preservatives.

In aqueous form, said pharmaceutical formulation may be ready for administration, while in lyophilised form said formulation can be transferred into liquid form prior to administration, e.g., by addition of water for injection which may or may not comprise a preservative such as for example, but not limited to, benzyl alcohol, antioxidants like vitamin A, vitamin E, vitamin C, retinyl palmitate, and selenium, the amino acids cysteine and methionine, citric acid and sodium citrate, synthetic preservatives like the parabens methyl paraben and propyl paraben.

Said pharmaceutical formulation may further comprise one or more stabilizer, which may be, e.g., an amino acid, a sugar polyol, a disaccharide and/or a polysaccharide. Said pharmaceutical formulation may further comprise one or more surfactant, one or more isotonizing agents, and/or one or more metal ion chelator, and/or one or more preservative.

The pharmaceutical formulation as described herein can be suitable for at least intravenous, intramuscular or subcutaneous administration. Alternatively, said conjugate according to the present invention may be provided in a depot formulation which allows the sustained release of the biologically active agent over a certain period of time.

In still another aspect of the present invention, a primary packaging, such as a prefilled syringe or pen, a vial, or an infusion bag is provided, which comprises said formulation according to the previous aspect of the invention.

The prefilled syringe or pen may contain the formulation either in lyophilised form (which has then to be solubilised, e.g., with water for injection, prior to administration), or in aqueous form.

Said syringe or pen is often a disposable article for single use only, and may have a volume between 0.1 and 20 ml. However, the syringe or pen may also be a multi-use or multi-dose syringe or pen.

Said vial may also contain the formulation in lyophilized form or in aqueous form and may serve as a single or multiple use device.

According to one embodiment, the present invention provides a composition comprising
at least one immune checkpoint inhibitor, and
at least one conjugate according to the invention as disclosed herein above.

In the context of the present invention, the term “immune checkpoint inhibitor” or simply “checkpoint inhibitor” or “ICI” refers to any agent or compound that, either directly or indirectly, decreases the level of or inhibits the function of an immune checkpoint receptor protein or molecule found on the surface of an immune cell (for example, a T cell), or to any agent or compound that, either directly or indirectly, decreases the level of or inhibits the function of a ligand that binds to said immune checkpoint receptor protein or molecule, either as a soluble compound or on the surface of an immune cell-inhibitory cell. Such an inhibitory cell can be, for example, a cancer cell, a regulatory T cell, a tolerogenic antigen presenting cell, a myeloid-derived suppressor cells, a tumor-associated macrophage, or a cancer-associated fibroblast.

Said ligand is typically capable of binding the immune checkpoint receptor protein or molecule on the immune cell. A non-limiting example of an immune checkpoint receptor protein-ligand pair is PD-1, PD-L1. PD-1 is an immune checkpoint receptor protein found on T-cells. PD-L1, which can be over-expressed by cancer cells, binds to PD-1 and helps the cancer cells to evade the host immune system attack. Accordingly, an immune checkpoint inhibitor prevents the PD-1/PD-L1 interaction by either blocking the PD-1 on the T cell (i.e., acts as a PD-I inhibitor) or the PD-L1 on the cancer cell (i.e., acts as a PD-L1 inhibitor), thereby maintaining or restoring anti-tumor T-cell activity or blocking inhibitory cancer cell activity. Other non-limiting examples of immune checkpoint inhibitors include CTLA-4, LAG-3, TIM-3, TIGIT, VISTA, OX40, GITR, ICOS, CD276 (B7-H3), B7-H4 (VTCN1), IDO, KIR, CD122, CD137, CD94/NKG2A, CD80, CD86, Galectin-3, LSECtin, CD112, Ceacam-1, Gal-9, PtdSer, HMGB1, HVEM, CD155 and BTLA (CD272). Corresponding compositions and their respective use are disclosed in WNO2022/096604.

According to one embodiment, the immune checkpoint inhibitor according to the invention is an antibody selected from the group consisting of nivolumab, pidilizumab, pembrolizumab, atezolizumab, avelumab, durvalumab, cemiplimab, ipilimumab, PD-1, PD-2, PD-3, PD-4, or PD-5, tremelimumab or an antigen-binding fragment thereof, or an antigen-binding derivative thereof, preferably wherein the antibody is avelumab, pembrolizumab, nivolumab, or ipilimumab or an antigen-binding fragment thereof, or an antigen-binding derivative of said antibodies.

Nivolumab (CAS Registry Number 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WN02006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WNO2009/101611.

Pembrolizumab (formerly also known as lambrolizumab; trade name Keytruda; also known as MK-3475) disclosed e.g. in Hamid, O. et al. (2013) New England Journal of Medicine 369(2):134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WNO2009/114335. It is approved by the FDA for the treatment of patients suffering from unresectable or metastatic melanoma and patients with metastatic NSCLC.

Atezolizumab is a fully humanized, engineered monoclonal antibody of IgG1 isotype against PD-L) as disclosed WO2010/077634.

Avelumab is a human IgG1 anti-PD-L1 monoclonal antibody as disclosed in VN02013/079174 A1.

Durvalumab is a human immunoglobulin G1 kappa (IgG1K) anti-PD-L1 monoclonal antibody as disclosed in WO2011/066389 A1.

Cemiplimab is a human IgG4 monoclonal antobody which binds to PD-1 thereby blocking its interaction with PD-L1 and PD-L2. Cemiplimab is disclosed in WO15112800 A1 as H4H7798N.

Ipilumumab (CAS Registry Number 477202-00-9, which may also be referred to as 10D1, or MDX010, MDX-101) is a human IgG1 antibody that binds Cytotoxic T-lymphocyte antigen-4 (CTLA4). CTLA-4 is an inhibitory molecule that competes with the stimulatory CD28 for binding to B7 on antigen presenting cells. CTLA-4 and CD28 are both presented on the surface of T-cells. Ipilimumab is a human IgG1 that binds CTLA-4, preventing the inhibition of T-cell mediated immune responses to tumors. Ipilimumab is e.g. disclosed in WO 01/14424 as antibody “10D1”.

PD1-1 to PD1-5 refer to anti-PD-1 antibodies as disclosed in WNO2018/220169.

Tremelimumab (which may also be referred to as ticilimumab, CP-675, CP-675,206, CAS Registry number 745013-59-6) is a fully human anti-CTLA4 antibody.

The INNs as used herein are meant to also encompass all biosimilar antibodies of the corresponding originator antibody as disclosed herein, including but not limited to those biosimilar antibodies authorized under 42 USC § 262 subsection (k) in the US and equivalent regulations in other jurisdictions.

In one embodiment, the invention pertains to the conjugate of the invention as disclosed herein, or the pharmaceutical composition as disclosed herein, or to the composition of the invention for use in the treatment of cancer, wherein the cancer is selected from gastrointestinal cancer, colorectal cancer, metastatic colorectal cancer (mCRC), stomach cancer, pancreatic cancer e.g. pancreatic adenocarcinoma, or liver cancer. The term “treating” or the phrase “to treat” as used herein refers to any type of treatment that imparts a benefit to a patient afflicted with a disease, such as cancer, more particularly gastrointestinal cancer, colorectal cancer, metastatic colorectal cancer (mCRC), or pancreatic cancer, including improvement in the condition of the patient (e.g., in one or more symptoms), and/or delay in the progression of the condition.

According to one embodiment, the gastrointestinal cancer, colorectal cancer, metastatic colorectal cancer (mCRC), stomach cancer, pancreatic cancer e.g. pancreatic adenocarcinoma, or liver cancer as disclosed herein is characterized by a deletion, or translocation of chromosome 17p13.1, whereby the deletion may e.g. be a hemizygous deletion of chromosome 17.p13.1 Accordingly, the the gastrointestinal cancer, colorectal cancer, metastatic colorectal cancer (mCRC), stomach cancer, pancreatic cancer e.g. pancreatic adenocarcinoma, or liver cancer as disclosed herein to be treated with the conjugate, pharmaceutical composition, or the composition of the invention as disclosed herein are characterized by a deletion, or hemizygous deletion of chromosome 17 p13.1.

According to one embodiment, the gastrointestinal cancer, colorectal cancer, metastatic colorectal cancer (mCRC), stomach cancer, pancreatic cancer (e.g. pancreatic adenocarcinoma), or liver cancer as disclosed herein which may e.g. be treated with the conjugate, pharmaceutical composition, or composition of the invention as disclosed herein, are characterized by a hemizygous loss of the POLR2A gene, or of the TP53 and POLR2A genes. The term “hemizygous” as used according to the invention refers to an individual or cell which has only one full allele of a gene or chromosome segment rather than the usual two. A hemizygote refers to a cell or organism whose genome includes only one full allele at a given locus, whether the allele is wildtype or mutant, e.g. the cells of the gastrointestinal cancer, colorectal cancer, metastatic colorectal cancer (mCRC), stomach cancer, pancreatic cancer as disclosed herein are hemizygotes for chromosome locus 17p13, preferably the cells of the gastrointestinal cancer, colorectal cancer, metastatic colorectal cancer (mCRC), stomach cancer, pancreatic cancer as disclosed above are hemizygotes for the genes TP53 and POLR2A. “TP53” as used herein refers “tumor protein 53” gene which encodes a tumor suppressor protein (P53) which comprises transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism. Mutations in this gene are associated with a variety of human cancers, including hereditary cancers such as Li-Fraumeni syndrome.

The tumour suppressor gene TP53 is frequently inactivated by mutation or deletion in a majority of human tumors. “POLR2A” as used herein refers to the POLR2A gene which encodes the largest subunit of the human RNA polymerase II complex and which is indispensable for the polymerase activity in mRNA synthesis. Hemizygous loss of chromosome 17p13, e.g. del(17p13.1), may be detected by fluorescence in situ hybridization (FISH) as disclosed in Merz et al. Am J Hematol. 2016 November; 91(11):E473-E477.

The cells of the gastrointestinal cancer, colorectal cancer, metastatic colorectal cancer (mCRC), stomach cancer, pancreatic cancer as disclosed herein may e.g. not be a homogenous group of cells with regard to the loss of TP53 and/or POLR2A. For example, from about 5%, 7.5%, 10%, 15%, 20%, 25%, 30%, 40% 50%, 60% to about 70%, 75%, 80%, 85%, 90%, 95%, 100, or from about 70%, 75%, 80, 85% to about 90%, 92.5%, 95%, 97.5%, 100% of the cancer cells as disclosed above may be hemizygous for the del(17p13.1), TPS3 and/or POLR2A, or e.g. at least 5%, 10%, 15%, 20%, 25%, 30%, 40%, 45%, 50%, 60%, 70%, 75%, 80%, 90%, 95% of the cancer cells as disclosed herein are hemizygous for del(17p13), or for TPS3 and/or POLR2A.

The conjugate, pharmaceutical composition, or composition of the invention for use in the treatment of gastrointestinal cancer, colorectal cancer, metastatic colorectal cancer (mCRC), stomach cancer, pancreatic cancer which are characterized by a hemizygous loss of chromosome 17p13.1, or TPS3 and/or POLR2A may e.g. be particularly advantageous, since cells characterized by a hemizygous loss of chromosome 17p13.1, TPS3 and/or POLR2A are at least 10-fold, 25-fold, 50-fold, 100-fold, 250-fold, 500-fold 1000-fold more sensitive to the conjugate, pharmaceutical composition, or composition of the invention as disclosed herein. Accordingly, it may e.g. be beneficial to determine whether the cells of the cancer as disclosed herein comprise or consist of cells which are hemizygous for the loss of TPS3 and/or POLR2A, since at least 10-fold, 25-fold, 50-fold, 100-fold, 250-fold, 500-fold or 1000-fold less of the conjugate, pharmaceutical composition, or composition of the invention as disclosed herein may be used to achieve the desired therapeutic effect. Assays to assess the sensitivity of the cancer cells to the conjugate, pharmaceutical composition, or composition of the invention as disclosed herein can e.g. be done as described in Liu et al., Nature. 2015 April 30; 520(7549): 697-701.

According to one embodiment, the conjugate, pharmaceutical composition, or the composition comprising the conjugate of the invention and an immune checkpoint inhibitor according to the invention are for use in the treatment of a solid tumor in a patient, wherein the solid tumor of said patient is characterized by the expression of GUCY2C on the surface of the cancer cells of said solid tumor. Accordingly, the tumor cells express e.g. on their surface amino acids 24-430 of SEQ ID NO.: 76, or any fragment thereof that is recognized and specifically bound by the antibody, antibody fragment, or conjugate of the invention as disclosed herein. The term “specifically binds” or “specifically binding” as used herein refers to the binding of a antibody or conjugate of the invention as disclosed herein, having a Kd of at least about 10⁻⁶M, 10⁻⁷ M, 10⁻⁶M, or from about 10⁻⁶M to about 10⁻⁹ M, 10⁻¹⁰M, 10⁻¹¹M, 10⁻¹²M, or of about 5×10⁻⁶ M, 5×10⁻¹⁰M to about 2.5×10⁻¹¹M, 5×10⁻¹¹M, 2.5×10⁻¹²M, 5×10⁻¹²M to its antigen, such as e.g. an epitope of human GUCY2C. The term “epitope” as used herein refers to the part of a macromolecule, preferably a polypeptide, such as human GUCY2C, that is recognized by antigen-binding molecules, such as the antibody, or antigen-binding fragment thereof, or antigen-binding derivative thereof of the invention as disclosed herein, and more particularly by the antigen-binding site of said molecules. Epitopes define the minimum binding site for an antibody molecule, and thus represent the target of specificity of an antibody molecule. Epitopes can be further defined as structural epitopes or functional epitopes. A “structural epitope” consists of amino acids or other molecules in a region that is in close contact with the antibody usually revealed by a structure. A “functional epitope” is defined, as those parts of a molecule that make an energetic contribution to binding such that when they are changed there is a decrease in binding affinity. Therefore, the residues making contact with the paratope, and what residues that are contributing to the affinity, whether they are proximal or not are important considerations when defining the epitope. Structural epitopes may e.g. be a linear continuous sequence of about 5 amino acids to about 50, 100 amino acids in length, or a conformational epitope which is formed by the three dimensional structure of the polypeptide and which may comprise discontinuous amino acids of the polypeptide. The affinity of the anti-GUCY2C antibody, or of the conjugate of the invention can be measured using well know methods, for example in an in vitro assay using plasmon resonance (BIAcore, GE-Healthcare Uppsala, Sweden).

The solid tumor as disclosed above may, e.g. also be characterized by a hemizygous loss of the POLR2A gene, or of the TP53 and POLR2A genes. For example, from about 5%, 7.5%, 10%, 15%, 20%, 25%, 30%, 40% 50%, 60% to about 70%, 75%, 80%, 85%, 90%, 95%, 100, or from about 70%, 75%, 80, 85% to about 90%, 92.5%, 95%, 97.5%, 100% of the cells of the solid tumor may be hemizygous for the del(17p13.1), TPS3 and/or POLR2A, or e.g. at least 5%, 10%, 15%, 20%, 25%, 30%, 40%, 45%, 50%, 60%, 70%, 75%, 80%, 90%, 95% of the cells of the solid tumor as disclosed above are hemizygous for del(17p13), or for TPS3 and/or POLR2A.

According to one embodiment, the invention also pertains to antibodies that compete with the antibody of the invention for binding to human GUCY2C. Antibodies that compete with the antibody of the invention for binding to human GUCY2C may e.g. be determined using an Octet HTX biosensor (Pall ForteBio Corp.) in analogous fashion as disclosed in WO2015/112800, or e.g. as disclosed in Syedbasha et al., J Vis Exp. 2016; (109): 53575, the content of which is hereby incorporated in its entirety.

According to one embodiment, the conjugate, or the pharmaceutical composition according to the invention as disclosed herein are for use in the treatment of colorectal cancer, metastatic colorectal cancer(mCRC), stomach cancer, pancreatic cancer, or liver cancer in a patient, wherein the patient did not respond to a treatment, e.g. first-line or second-line treatment, comprising cetuximab, or panitumumab alone, or in combination with 5-fluorouracil (5-FU) and oral capecitabine (CAP), FOLFOX (5-FU and oxaliplatin), FOLFIRI (5-FU and irinotecan), XELOX/CAPOX (CAP and oxaliplatin), CAPIRI (CAP and irinotecan), or FOLFIRINOX (5-fluorouracil, leucovorin, irinotecan, and oxaliplatin). As used herein, the term “first-line therapy” or “first-line treatment” or “first-line therapy” means a therapy or treatment for which its label does not include a requirement or recommendation that said therapy or treatment should be used only after other therapies or treatments were shown to be unsatisfactory or unsuccessful.

It can also include a therapy and/or treatment wherein no other actives (beyond the main active) are administered to the individual subject in need. The term “second-line treatment” or “second line therapy” refers to a treatment for a disease or condition after the initial treatment (first-line treatment) has failed, stopped working, or has side effects that are not tolerated due to severe side effects and associated loss of quality of life.

According to some embodiments, the conjugate, or the pharmaceutical composition of the invention, alone or optionally in combination with an immune checkpoint inhibitor as disclosed above, are e.g. for use in the treatment of colorectal cancer, metastatic colorectal cancer(mCRC), stomach cancer, pancreatic cancer, or liver cancer in a patient for which standard of care treatment options have failed, e.g. failed first and/or second-line or even third line treatment. For example, the conjugate or the pharmaceutical composition of the invention may e.g. be for use in a patient afflicted with CRC, who has failed the above treatment options, or who has failed a third line treatment using the combination drug trifluridine/tipiracil (TAS-102) following a treatment using fluoropyrimidine-, oxaliplatin-, and irinotecan-based chemotherapy, anti-VEGF therapy, and, for RAS-wild type tumors, anti-EGFR therapy.

According to one embodiment, the conjugate (anti-GUCY2C conjugate), or the pharmaceutical composition of the invention are for use in the treatment of colorectal cancer, or metastatic colorectal cancer(mCRC), wherein the colorectal cancer, or metastatic colorectal cancer (mCRC) is characterized by a KRAS mutation and/or a BRAF mutation. KRAS mutations according to the invention include without limitation KRAS mutations G13D, G12C, G12V, and G12D. BRAF mutations according to the invention include without limitation BRAF mutations V600E, V600K or V600R. Accordingly, the conjugate, or the pharmaceutical composition of the invention are for use in the treatment of CRC, or mCRC that are characterized by having one of the following mutations KRAS G12C, KRAS G12V, KRAS G12D, G13D and/or BRAF V600E, BRAF V600K, or BRAF V600R.

According to some embodiments, the conjugate, or the pharmaceutical composition of the invention as disclosed above for use in the treatment of colorectal cancer, metastatic colorectal cancer(mCRC), stomach cancer, pancreatic cancer, or liver cancer may be combined with one or more immune checkpoint inhibitors as disclosed above, such as e.g. anti-PD-1 (e.g. nivolumab, PD-1-PD-5, pidilizumab, or pemprolizumab) or anti-PD-L1 (e.g., avelumab), anti-CTLA-4 (e.g. ipilimumab, or tremelimumab) checkpoint inhibitors, or e.g. with a KRAS inhibitor, such as BI 1823911, sotorasib, adagrasib, LY3537982, JNJ-74699157, or KRAS-SOS1 inhibitors such as e.g. B11701963, or BRAF inhibitors such as e.g. Vemurafenib, Dabrafenib, or Encorafenib, or combinations of Dabrafenib and trametinib, vemurafenib and cobimetinib, or Encorafenib and binimetinib, or combinations of the aforementioned checkpoint inhibitors with a KRAS inhibitor, or BRAF inhibitor.

In one embodiment, the present invention pertains to the use of the conjugate or the pharmaceutical composition of the invention in the manufacture of a medicament for the treatment of gastrointestinal cancer, colorectal cancer, metastatic colorectal cancer (mCRC), stomach cancer, pancreatic cancer (e.g. pancreatic adenocarcinoma), liver cancer, or the treatment of a GUCY2C-positive or GUCY2C-expressing solid tumor.

In another aspect, the present invention pertains to a method of treating a patient afflicted with with gastrointestinal cancer, wherein the method comprises administering to said patient a therapeutically effective amount of the conjugate, or of the pharmaceutical composition of the invention alone, or in combination with further pharmacologically active compounds, such as without limitation one or more immune checkpoint inhibitors as disclosed above, such as e.g. anti-PD-1 (e.g. nivolumab, PD-1-PD-5, pidilizumab, or pemprolizumab) or anti-PD-L1 (e.g., avelumab), anti-CTLA-4 (e.g. ipilimumab, tremelimumab) checkpoint inhibitors, or e.g. with a KRAS inhibitor, such as BI 1823911, sotorasib, adagrasib, LY3537982, JNJ-74699157, or KRAS-SOS1 inhibitors such as e.g. B11701963, or BRAF inhibitors such as e.g. vemurafenib, dabrafenib, or encorafenib, or combinations of dabrafenib and trametinib, vemurafenib and cobimetinib, or encorafenib and binimetinib, or combinations of the aforementioned checkpoint inhibitors with a KRAS inhibitor, or BRAF inhibitor.

In one embodiment, the invention pertains to a method of treating a patient afflicted with gastrointestinal cancer, wherein the method comprises administering to said patient a therapeutically effective amount of the conjugate, or of the pharmaceutical composition as disclosed above, wherein the patient did not respond to a treatment, e.g. first- or second-line treatment, comprising cetuximab, or panitumumab alone, or in combination with 5-fluorouracil (5-FU) and oral capecitabine (CAP), FOLFOX (5-FU and oxaliplatin), FOLFIRI (5-FU and irinotecan), XELOX/CAPOX (CAP and oxaliplatin), CAPIRI (CAP and irinotecan), FOLFIRINOX (5-fluorouracil, leucovorin, irinotecan, and oxaliplatin) or e.g. wherein the patient has failed a third line treatment using the combination drug trifluridine/tipiracil (TAS-102) following a treatment using fluoropyrimidine-, oxaliplatin-, and irinotecan-based chemotherapy, anti-VEGF therapy, and, for RAS-wild type tumors, anti-EGFR therapy. Accordingly, the above method of treatment may e.g. be used in the treatment of patients afflicted with refractory colorectal cancer, or refractory metastatic colorectal cancer (mCRC), stomach cancer, pancreatic cancer, or liver cancer.

In some embodiments, the colorectal cancer, metastatic colorectal cancer(mCRC), stomach cancer, pancreatic cancer, or liver cancer to be treated in the inventive method of treatment is characterized by a hemizygous loss of TP53, POLR2A, or del(17p13).

In some embodiments, the colorectal cancer, metastatic colorectal cancer(mCRC), stomach cancer, pancreatic cancer, or liver cancer to be treated in the inventive method of treatment is characterized by a KRAS mutation and/or a BRAF mutation as disclosed herein, e.g.

KRAS mutations include the mutations G13D, G12C, G12V, and G12D, or BRAF mutations BRAF mutations V600E, V600K or V600R.

### REFERENCES

Altschul et al., 1990, J. Mol. Biol. 215, 403-410, “Basic local alignment search tool”
Altschul et al. (1997), Nucleic Acids Res, 25:3389-3402), “Gapped BLAST and PSI-BLAST: a new generation of protein database search programs”
Armour, et al., Eur. J. Immunol. 29(8) (1999) 2613-2624; ,,Recombinant human IgG molecules lacking Fcgamma receptor I binding and monocyte triggering activities”
Arnold et al. (2020) Gastroenterology 159:335-349, “Global Burden of 5 Major Types of Gastrointestinal Cancer”
Barber et al N Engl J Med (2004) 351: 2883, “Somatic mutadons of EGFR in colorectal cancers and glioblastomas”
Carter, Biochem. J. (1986) Vol. 237: 1-7, “Site-directed mutagenesis”
Chong Z X, et al. (2021) PeerJ 9:e11165, “Transfecdon types, methods and strategies: a technical review”
Compendium of Chemical Terminology” (“Gold Book”) published by the International Union of Pure and Applied Chemistry (IUPAC) version 2.3.3, goldbook.iupac.org, ISBN: 0-9678550-9-8
Chothia et al. (1989) Nature 342: 877-883, “Conformations of immunoglobulin hypervariable regions”
Danaee et al (2017) PLoS One. 2017; 12(12): e0189953), “Consistent expression of guanylyl cyclase-C in primary and metastatic gastrointestinal cancers”
Davis et al. (2002) Chem. Rev. 102: 579-602, “Synthesis of Glycoproteins”
De' Angelis et al. Acta Biomed 2018 Dec. 17; 89(9-S):97-101, “Microsatellite instability in colorectal cancer”
Devereux et al, 1984, Nucleic Acids Res., 387-395, “A comprehensive set of sequence analysis programs for the VAX”
Doman et al (2009) Blood 114(13):2721-2729, “Therapeutc potental of an anti-CD79b antibody-drug conjugate, anti-CD79b-vc-MMAE, for the treatment of non-Hodgkin lymphoma”
Dubowchik and Walker (1999) Pharm. Therapeutics 83:67-123; “Receptor-mediated and enzyme-dependent targeing of cytotoxic anticancer drugs”
Edelman et al., 1969, Proc Natl Acad Sci USA 63:78-85, “The covalent structure of an entire gammaG immunoglobulin molecule”
Fishwild (1996) Nature Biotechnol. 14: 845-851, “High-avidity human IgG_{K} monoclonal antibodies from a novel strain of minilocus transgenic mice”
Fus-Kujawa A. et al (2021) Front Bioeng. Biotechnol. 9:701031, “An Overview of Methods and Tools for Transfection of Eukaryotic Cells in vitro.”
Ghetie et al. (2000) Annu. Rev. Immunol. 18: 739-766, “Multiple roles for the major histocompatibility complex class I-related receptor FcRn”
Green (1999) J. Immunol. Methods 231: 11-23, “Antibody engineering via genetic engineering of the mouse: XenoMouse strains are a vehicle for the facile generation of therapeutic human monoclonal antibodies”
Hamid, O. et al. (2013) New England Journal of Medicine 369(2):134-44, “Safety and Tumor Responses with Lambrolizumab (Anti-PD-1) in Melanoma”
Hang et al. (2001) Acc. Chem. Res 34: 727-736, “Chemoselecdve approaches to glycoprotein assembly”
Huang et al. MAbs. 2018 January; 10(1): 95-103, “Hydrogen/deuterium exchange mass spectometry and computational modeling reveal a discontinuous epitope of an antibody/TL1A Interaction”
Jain et al. (2015) Pharm Res 32:3526-40, “Current ADC Linker Chemistry”
Jethva and Gross, Front. Anal. Sci., 18 May 2023, “Hydrogen deuterium exchange and other mass spectometry-based approaches for epitope mapping”
Joosten et al Gastroenterology 2019 October; 157(4):1153-1155, “MET Signaling Overcomes Epidermal Growth Factor Receptor Inhibition in Normal and Colorectal Cancer Stem Cells Causing Drug Resistance”
Junghans (1997) Immunol. Res 16: 29-57; “Finally! The Brambell receptor (FcRB). Mediator of transmission of immunity and protection from catabolism for IgG”
Junutula, et al., 2008 Nature Biotech., 26(8):925-932, “Site-specific conjugation of a cytotoxic drug to an antibody improves the therapeutic index”
Karlin et al. (1993), PNAS USA, 90:5873-5877, “Applications and statistics for multiple high-scoring segments in molecular sequences”
Kabat et al., Sequences of Proteins of Immunological Interest, 4th Ed., U.S. Department of Health and Human Services, National Institutes of Health (1987)
Kozbor (1984) J. Immunol 133, 3001, “A human hybrid myeloma for production of human monoclonal antibodies.”
Kwong and Rader, Curr. Protoc. Protein Sci. 55:6.10.1-6.10.14, “E. coli Expression and Purification of Fab Antibody Fragments”
Usby et al., “GUCY2C as a biomarker to target precision therapies for patients with colorectal cancer”
Uu et al., Nature. 2015 April 30; 520(7549): 697-701, “TP53 loss creates therapeutic vulnerability in colorectal cancer”
Mendez (1997) Nature Genetics, 15: 146-156, “Functional transplant of megabase human immunoglobulin loci recapitulates human antibody response in mice”
Neville et al. (1989) Biol. Chem. 264: 14653-61, “Enhancement of immunotoxin efficacy by acid-cleavable cross-linking agents utilizing diphtheria toxin and toxin mutants”
Magee et al. Cancer Immunol Res. 6(5), 509-516 (2018, “Human GUCY2C-Targeted Chimeric Antigen Receptor (CAR)-Expressing T Cells Eliminate Colorectal Cancer Metastases”
Merz et al. Am J Hematol. 2016 November; 91(11):E473-E477, “Baseline characteristics, chromosomal alterations, and treatment affecting prognosis of deletion 17p in newly diagnosed myeloma”
Pearson (1990), Methods Enzymol. 83, 63-98, “Rapid and sensitive sequence comparison with FASTP and FASTA”
Pearson and Lipman (1988), Proc. Natl. Acad. Sci. U.S.A 85, 2444-2448, “Improved tools for biological sequence comparison”
Rathanaswami et al. Analytical Biochemistry, Vol. 373:52-60, 2008, “High-affinity binding measurements of antibodies to cell-surface-expressed antigens”
Riechmann et al., Nature 332:323-7, 1988, “Reshaping human antibodes for therapy”
Sambrook and Russel “Molecular Cloning—A laboratory manual”, 4^{th} edition (2001), ISBN 978-1-936113-42-2, chapter 15, pages 1131-1203.
Sears et al. (2001) Science 291: 2344; “Toward automated synthesis of oligosaccharides and glycoproteins”
Shields, et al., J. Biol. Chem. 276(9) (2001) 6591-6604, “High resolution mapping of the binding site on human IgG1 for Fc gamma RI, Fc gamma RII, Fc gamma RIII, and FcRn and design of IgG1 variants with improved binding to the Fc gamma R”
Smith and Waterman (1981), J. Mol. Biol. 147, 195-197, “Identification of common molecular subsequences”
Syedbasha et al., J Vis Exp. 2016; (109): 53575, “An ELISA Based Binding and Competition Method to Rapidly Determine Ligand-receptor Interactions”
Tomizuka (2000) PNAS 97: 722-727, “Double trans-chromosomic mice: Maintenance of two individual human chromosome fragments containing Ig heavy and K loci and expression of fully human antibodies”
Thompson et al, Nucleic Acids Res. 1994 Nov. 11; 22(22):4673-80.
Voss et al. BMC Molecular Biology 2014, 15:7, “A novel, non-radioactive eukaryotic in vitro transcription assay for sensitive quantification of RNA polymerase I activity”
Wacker et al. (2002) Science 298: 1790; “N-Linked Glycosylation in Campylobacter jejuni and Its Functional Transfer into E. coli”
Weland T, Faulstich H., CRC Crit Rev Biochem. 5 (1978) 185-260, “Amatoxins, phallotoxins, phallolysin, and antamanide: the biologically active components of poisonous Amanita mushrooms”
Wilkinson et al. PLoS One. 2021; 16(12): e0260954, “Fc-engineered antibodies with immune effector functions completely abolished”
Winter (1994) Annu. Rev. Immunol 12: 433-455, “Making antibodies by phage display technology”
Womble Methods Mol Biol. 2000; 132:3-22, “GCG: The Wisconsin Package of sequence analysis programs
Zhang et al. (2004) Science 303: 371-373, “A new strategy for the synthesis of glycoproteins”

### PATENT LITERATURE

U.S. Pat. Nos. 5,122,368;
U.S. Pat. No. 5,824,805;
U.S. Pat. No. 5,622,929
U.S. Pat. No. 5,622,929;
WO2016040856A2
U.S. Pat. Nos. 7,521,541;
U.S. Pat. No. 7,723,485;
WO2009/052249
WO2016/142049
VN003/018771
WO2013/163633
WO2021/205325 A1
WO 2021/234402 A2
WO2016142049A1
WO03026577
WO2005/112919
WO2022/096604
U.S. Pat. No. 8,008,449
WO2006/121168
U.S. Pat. No. 8,354,509
WO2009/114335
WO2009/101611.
U.S. Pat. No. 8,354,509
WO2009/114335
WO2010/077634
WO2013/079174 A1
WO2011/066389 A1.
WO015112800 A1
WO015112800 A1
WO 01/14424
WO2018/220169.
WO2015/112800
U.S. Pat. Nos. 5,530,101;
U.S. Pat. No. 5,585,089;
U.S. Pat. No. 5,693,761;
U.S. Pat. No. 5,693,762;
U.S. Pat. No. 6,180,370.

### Sequences

TABLE 1
Amino acid sequences of the invention

| SEQ ID NO | Sequence | Description |
|---|---|---|
| 1 | DIRMTQSPSSLSASAGDRVTITC | mAb 8 LC FR1 |
| 2 | RASQSISSYLN | mAb 8 LC CDR1 |
| 3 | WYQQKPGKAPKLLIY | mAb 8 LC FR2 |
| 4 | AASSLQS | mAb 8 LC CDR2 |
| 5 | GVPSRFSGSGSGTDFTLTISSLQPEDFATYYC | mAb 8 LC FR3 |
| 6 | QQSYSTPLT | mAb 8 LC CDR3 |
| 7 | FGGGTKLEIK | mAb 8 LC FR4 |
| 8 | GVPSRFSGSGSGTDFTLAISSLQPEDFATYYC | mAb 1 LC FR3 |
| 9 | QQSYSTPWT | mAb 1 LC CDR3 |
| 10 | FGQGTKVDIK | mAb 1 LC FR4 |
| 11 | QQSYSTPYT | mAb 24 LC CDR3 |
| 12 | FGQGTKVEIK | mAb 24 LC FR4 |
| 13 | QSVLTQPPSASGTPGQRVTISC | mAb 40 LC FR1 |
| 14 | SGSSSNIGS-NTVN | mAb 40 LC CDR1 |
| 15 | WYQQLPGTAPRLLIY | mAb 40 LC FR2 |
| 16 | SNRQRPS | mAb 40 LC CDR2 |
| 17 | VPDRFSGARSGTSASLAITGLQPEDEADYYC | mAb 40 LC FR3 |
| 18 | ATWDVGLRGRV | mAb 40 LC CDR3 |
| 19 | FGGGTKLTVL | mAb 40 LC FR4 |
| 20 | QSGLTQPRSVSGSPGQSVTISC | mAb 28 LC FR1 |
| 21 | TGTSSDVGGYKYVS | mAb 28 LC CDR1 |
| 22 | WYQQHPGKAPKLKIY | mAb 28 LC FR2 |
| 23 | DVSKRPS | mAb 28 LC CDR2 |
| 24 | GVPDRFSGSKSGNTASLTISGLQAEDEADYYC | mAb 28 LC FR3 |
| 25 | TSYTSSNTVV | mAb 28 LC CDR3 |
| 26 | FGGGTKVTVL | mAb 28 LC FR4 |
| 27 | QSALTQPÅSVSGSPGQSITISC | mAb 41 LC FR1 |
| 28 | TGTSSDVGSYNLVS | mAb 41 LC CDR1 |
| 29 | WYQQHPGKAPKLMIY | mAb 41 LC FR2 |
| 30 | EGSKRPS | mAb 41 LC CDR2 |
| 31 | GVSNRFSASKSGNTASLTISGLQTEDEADYYC | mAb 41 LC FR3 |
| 32 | SSYVPRSSLV | mAb 41 LC CDR3 |
| 33 | QVQLVQSGAEVKKPGASVKVSCKASGYIFI | mAb 1 HC FR1 |
| 34 | SSAMH | mAb 1 HC CDR1 |
| 35 | WVRQAPGQRLEWMG | mAb 1 HC FR2 |
| 36 | LINPGNGNTKYSQKFQG | mAb 1 HC CDR2 |
| 37 | RVTITRDTSASTAYMELSSLRSEDTAVYYCAR | mAb 1 HC FR3 |
| 38 | AYFRGLGFDI | mAb 1 HC CDR3 |
| 39 | WGQGTLVTVSS | mAb 1 HC FR4 |
| 40 | EVQLVQSGAEVKKPGASVKVSCKASGYTFT | mAb 40 HC FR1 |
| 41 | SYYMH | mAb 40 HC CDR1 |
| 42 | INPSGGSTSYAQKFQG | mAb 40 HC CDR2 |
| 43 | RVTMTRDTSTSTVYMELSSLRSEDTAVYYCAR | mAb 40 HC FR3 |
| 44 | VRQWPLASDY | mAb 40 HC CDR3 |
| 45 | QVQLVESGAEVKKPGASVKVSCKASGYTFT | mAb 28 HC FR1 |
| 46 | SYAIS | mAb 28 HC CDR1 |
| 47 | RIIPILGIANYAQKFQG | mAb 28 HC CDR2 |
| 48 | RVTITADKSTSTAYMELSRLRSDDTAVYYCAI | mAb 28 HC FR3 |
| 49 | DSSGWMTFDY | mAb 28 HC CDR3 |
| 50 | WGQGTMVTVSS | mAb 28 HC FR4 |
| 51 | QVQLVQSGAEVKKPGSSVKVSCKASGGTFS | mAb 41 HC FR1 |
| 52 | TYTIN | mAb 41 HC CDR1 |
| 53 | RVTITADKSTSTAYMELSSLRSEDTAVYYCAR | mAb 41 HC FR3 |
| 54 | DTPRLRSSYYMDV | mAb 41 HC CDR3 |
| 55 | WVRQAPGQGLEWMG | mAb 41 HC FR2 |
| 56 | DIQMTQSPSSLSASAGDRVTITCRASQSISSY | mAb 1 VL |
| | LNWYQQKPGKAPKLLIYAASSLQSGVPSRFSG | |
| | SGSGTDFTLAISSLQPEDFATYYCQQSYSTPW | |
| | TFGQGTKVDIK | |
| 57 | DIRMTQSPSSLSASAGDRVTITCRASQSISSY | mAb 8 VL |
| | LNWYQQKPGKAPKLLIYAASSLQSGVPSRFSG | |
| | SGSGTDFTLTISSLQPEDFATYYCQQSYSTPL | |
| | TFGGGTKLEIK | |
| 58 | DIQMTQSPSSLSASAGDRVTITCRASQSISSY | mAb 24 VL |
| | LNWYQQKPGKAPKLLIYAASSLQSGVPSRFSG | |
| | SGSGTDFTLAISSLOPEDFATYYCQQSYSTPY | |
| | TFGQGTKVEIK | |
| 59 | QSGLTQPRSVSGSPGQSVTISCTGTSSDVGGY | mAb 28 VL |
| | KYVSWYQQHPGKAPKLKIYDVSKRPSGVPDRF | |
| | SGSKSGNTASLTISGLQAEDEADYYCTSYTSS | |
| | NTVVFGGGTKVTVL | |
| 60 | QSVLTQPPSASGTPGQRVTISCSGSSSNIGSN | mAb 40 VL |
| | TVNWYQQLPGTAPRLLIYSNRQRPSGVPDRFS | |
| | GARSGTSASLAITGLQPEDEADYYCATWDVGL | |
| | RGRVFGGGTKLTVL | |
| 61 | QSALTQPASVSGSPGQSITISCTGTSSDVGSY | mAb 41 VL |
| | NLVSWYQQHPGKAPKLMIYEGSKRPSGVSNRF | |
| | SASKSGNTASLTISGLQTEDEADYYCSSYVPR | |
| | SSLVFGGGTKLTVL | |
| 62 | QVQLVQSGAEVKKPGASVKVSCKASGYIFISS | mAb 1 VH |
| | AMHWVRQAPGQRLEWMGLINPGNGNTKYSQKF | |
| | QGRVTITRDTSASTAYMELSSLRSEDTAVYYC | |
| | ARAYFRGLGFDIWGQGTLVTVSS | |
| 63 | QVQLVQSGAEVKKPGASVKVSCKASGYIFISS | mAb 8 VH |
| | AMHWVRQAPGQRLEWMGLINPGNGNTKYSQKF | |
| | QGRVTITRDTSASTAYMELSSLRSEDTAVYYC | |
| | ARAYFRGLGFDIWGQGTLVTVSS | |
| 64 | QVQLVQSGAEVKKPGASVKVSCKASGYIFISS | mAb 24 VH |
| | AMHWVRQAPGQRLEWMGLINPGNGNTKYSQKF | |
| | QGRVTITRDTSASTAYMELSSLRSEDTAVYYC | |
| | ARAYFRGLGFDIWGQGTLVTVSS | |
| 65 | QVQLVESGAEVKKPGASVKVSCKASGYTFTSY | mAb 28 VH |
| | AISWVRQAPGQGLEWMGRIIPILGIANYAQKF | |
| | QGRVTITADKSTSTAYMELSRLRSDDTAVYYC | |
| | AIDSSGWMTFDYWGQGTMVTVSS | |
| 66 | EVQLVQSGAEVKKPGASVKVSCKASGYTFTSY | mAb 40 VH |
| | YMHWVRQAPGQGLEWMGIINPSGGSTSYAQKF | |
| | QGRVTMTRDTSTSTVYMELSSLRSEDTAVYYC | |
| | ARVRQWPLASDYWGQGTLVTVSS | |
| 67 | QVQLVQSGAEVKKPGSSVKVSCKASGGTFSTY | mAb 41 VH |
| | TINWVRQAPGQGLEWMGRIIPVLGIANYAQKF | |
| | QGRVTITADKSTSTAYMELSSLRSEDTAVYYC | |
| | ARDTPRLRSSYYMDVWGQGTLVTVSS | |
| 68 | ASTKGPSVFPLAPSSKSTSGGTAALGCLVKDY | Heavy chain constant |
| | FPEPVTVSWNSGALTSGVHTFPAVLQSSGLYS | region (Wild type (WT)) |
| | LSSVVTVPSSSLGTQTYICNVNHKPSNTKVDK | |
| | KVEPKSCDKTHTCPPCPAPELLGGPSVFLFPP | |
| | KPKDTLMISRTPEVTCVVVDVSHEDPEVKFNW | |
| | YVDGVEVHNAKTKPREEQYNSTYRVVSVLTVL | |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 69 | ASTKGPSVFPLAPSSKSTSGGTAALGCLVKDY | Heavy chain constant |
| | FPEPVTVSWNSGALTSGVHTFPAVLQSSGLYS | region with L234A, |
| | LSSVVTVPSSSLGTQTYICNVNHKPSNTKVDK | L235A (LALA) mutations |
| | KVEPKSCDKTHTCPPCPAPEAAGGPSVFLFPP | (mutations in bold)* |
| | KPKDTLMISRTPEVTCVVVDVSHEDPEVKFNW | |
| | YVDGVEVHNAKTKPREEQYNSTYRVVSVLTVL | |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 70 | ASTKGPSVFPLAPSSKSTSGGTAALGCLVKDY | Heavy chain constant |
| | FPEPVTVSWNSGALTSGVHTFPAVLQSSGLYS | region with D265C |
| | LSSVVTVPSSSLGTQTYICNVNHKPSNTKVDK | mutation |
| | KVEPKSCDKTHTCPPCPAPELLGGPSVFLFPP | (mutation in bold)* |
| | KPKDTLMISRTPEVTCVVVCVSHEDPEVKFNW | |
| | YVDGVEVHNAKTKPREEQYNSTYRVVSVLTVL | |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 71 | ASTKGPSVFPLAPSSKSTSGGTAALGCLVKDY | Heavy chain constant |
| | FPEPVTVSWNSGALTSGVHTFPAVLQSSGLYS | region: modified Fc |
| | LSSVVTVPSSSLGTQTYICNVNHKPSNTKVDK | region with L234A, |
| | KVEPKSCDKTHTCPPCPAPEAAGGPSVFLFPP | L235A, D265C mutations |
| | KPKDTLMISRTPEVTCVVVCVSHEDPEVKFNW | (mutations in bold, |
| | YVDGVEVHNAKTKPREEQYNSTYRVVSVLTVL | underlined)* |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 72 | ASTKGPSVFPLAPSSKSTSGGTAALGCLVKDY | Heavy chain constant |
| | FPEPVTVSWNSGALTSGVHTFPAVLQSSGLYS | region: modified Fc |
| | LSSVVTVPSSSLGTQTYICNVNHKPSNTKVDK | region with L234A, |
| | KVEPKSCDKTHTCPPCPAPEAARGPSVFLFPP | L235A, G236R, D265C |
| | KPKDTLMISRTPEVTCVVVCVSHEDPEVKFNW | mutations (mutations in |
| | YVDGVEVHNAKTKPREEQYNSTYRVVSVLTVL | bold, underlined)* |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 73 | ASTKGPSVFPLAPSSKSTSGGTAALGCLVKDY | Heavy chain constant |
| | FPEPVTVSWNSGALTSGVHTFPAVLQSSGLYS | region: modified Fc |
| | LSSVVTVPSSSLGTQTYICNVNHKPSNTKVDK | region with L234G, |
| | KVEPKSCDKTHTCPPCPAPEGSRGPSVFLFPP | L235S, G236R, D265C |
| | KPKDTLMISRTPEVTCVVVCVSHEDPEVKFNW | mutations (mutations in |
| | YVDGVEVHNAKTKPREEQYNSTYRVVSVLTVL | bold, underlined)* |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 74 | ASTKGPSVFPLAPSSKSTSGGTAALGCLVKDY | Heavy chain constant |
| | FPEPVTVSWNSGALTSGVHTFPAVLQSSGLYS | region: modified Fc |
| | LSSVVTVPSSSLGTQTYICNVNHKPSNTKVDK | region with L234S, |
| | KVEPKSCDKTHTCPPCPAPESTRGPSVFLFPP | L235T, G236R, D265C |
| | KPKDTLMISRTPEVTCVVVCVSHEDPEVKFNW | mutations (mutations in |
| | YVDGVEVHNAKTKPREEQYNSTYRVVSVLTVL | bold, underlined)* |
| | HQDWLNGKEYKCKVSNKALPAPIEKTISKAKG | |
| | QPREPQVYTLPPSRDELTKNQVSLTCLVKGFY | |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 75 | ASTKGPSVFPLAPSSKSTSGGTAALGCLVKDY | Heavy chain constant |
| | FPEPVTVSWNSGALTSGVHTFPAVLQSSGLYS | region: modified Fc |
| | LSSVVTVPSSSLGTQTYICNVNHKPSNTKVDK | region with L234S, |
| | KVEPKSCDKTHTCPPCPAPESGRGPSVFLFPP | L235G, G236R, D265C |
| | KPKDTLMISRTPEVTCVVVCVSHEDPEVKFNW | mutations (mutations in |
| | YVDGVEVHNAKTKPREEQYNSTYRVVSVLTVL | bold, underlined, |
| | HQDWLNGKEYKCKVSNKALPAPIEKTISKAKG | numbering according to |
| | QPREPQVYTLPPSRDELTKNQVSLTCLVKGFY | Eu numbering system)* |
| | PSDIAVEWESNGQPENNYKTTPPVLDSDGSFF | |
| | LYSKLTVDKSRWQQGNVFSCSVMHEALHNHYT | |
| | QKSLSLSPGK | |
| 76 | MKTLLLDLALWSLLFQPGWLSFSQVSQNCHNG | mature GUCY2C with |
| | SYEISVLMMGNSAFAEPLKNLEDAVNEGLEIV | aa24-430 (extracellular |
| | RGRLQNAGLNVTVNATFMYSDGLIHNSGDCRS | domain) underlined; |
| | STCEGLDLLRKISNAQRMGCVLIGPSCTYSTE | UniProt entry no P25092 |
| | QMYLDTELSYPMISAGSFGLSCDYKETLTRLM | |
| | SPARKLMYFLVNFWKTNDLPFKTYSWSTSYVY | |
| | KNGTETEDCFWYLNALEASVSYFSHELGFKVV | |
| | LRQDKEFQDILMDHNRKSNVIIMCGGPEFLYK | |
| | LKGDRAVAEDIVIILVDLFNDQYFEDNVTAPD | |
| | YMKNVLVLTLSPGNSLLNSSFSRNLSPTKRDF | |
| | ALAYLNGILLFGHMLKIFLENGENITTPKFAH | |
| | AFRNLTFEGYDGPVTLDDWGDVDSTMVLLYTS | |
| | VDTKKYKVLLTYDTHVNKTYPVDMSPTFTWKN | |
| | SKLPNDITGRGPQILMIAVFTLTGAVVLLLLV | |
| | ALLMLRKYRKDYELRQKKWSHIPPENIFPLET | |
| | NETNHVSLKIDDDKRRDTIQRLRQCKYDKKRV | |
| | ILKDLKHNDGNFTEKQKIELNKLLQIDYYNLT | |
| | KFYGTVKLDTMIFGVIEYCERGSLREVLNDTI | |
| | SYPDGTFMDWEFKISVLYDIAKGMSYLHSSKT | |
| | EVHGRLKSTNCVVDSRMVVKITDFGCNSILPP | |
| | KKDLWTAPEHLRQANISQKGDVYSYGIIAQEI | |
| | ILRKETFYTLSCRDRNEKIFRVENSNGMKPFR | |
| | PDLFLETAEEKELEVYLLVKNCWEEDPEKRPD | |
| | FKKIETTLAKIFGLFHDQKNESYMDTLIRRLQ | |
| | LYSRNLEHLVEERTQLYKAERDRADRLNFMLL | |
| | PRLVVKSLKEKGFVEPELYEEVTIYFSDIVGF | |
| | TTICKYSTPMEVVDMLNDIYKSFDHIVDHHDV | |
| | YKVETIGDAYMVASGLPKRNGNRHAIDIAKMA | |
| | LEILSFMGTFELEHLPGLPIWIRIGVHSGPCA | |
| | AGVVGIKMPRYCLFGDTVNTASRMESTGLPLR | |
| | IHVSGSTIAILKRTECQFLYEVRGETYLKGRG | |
| | NETTYWLTGMKDQKFNLPTPPTVENQQRLQAE | |
| | FSDMIANSLQKRQAAGIRSQKPRRVASYKKGT | |
| | LEYLQLNTTDKESTYF | |
| 77 | MKTLLLDLVLWSLLFQPEWLYLTSQVSQNCHN | Guanylate cyclase 2C, |
| | GSYEISVLMMDNSAFAEPLENVEDAVNEGLEI | Macaca fascicularis, |
| | VRGRLQNAGLNVTVNASFMYSDGLIHNSGDCR | UniProt entry G7PJX5; |
| | SSTCEGLDLLRKISNAKRMGCVLMGPSCTYST | extracellular domain |
| | FQMYLDTELSYPMISAGSFGLSCDYKETLTRL | aa23-430 underlined |
| | MSPARKLTYFLVNFWKTNDLPFKTYSWSTSYV | |
| | YKNGTESEDCFWYLNALEASVSYFSHELSFKL | |
| | VLRQDKEFQDILMDHNRKSNVIVMCGDPEFLY | |
| | KLKGDRAVAEDIVIILVDLFNDQYFEDNVTAP | |
| | DYMKNVLVLTRSPGNSLLNSSFSRNLSPTKRD | |
| | FALAYLNGILLFGHMLKTFLENGENITTPKFA | |
| | HAFRNLTFEGYDGPVTLDDWGDVDSTMVLLYT | |
| | SVDTKKYKVLLTYDTHVNQTNPVDMSPTFTWK | |
| | NSKLPNDITDRGPQILMIAVFTLTGAVVLLLL | |
| | VALLMLRKYKKDYELRQKKWSHIPPENIFPLE | |
| | TNETNHVSLKIDDDKRRDTIQRLRQCKYDKKR | |
| | VILKDLKHNDGNFTEKQKIELNKLLQIDYYNL | |
| | TKFYGTVKLDTMIFGVIEYCERGSLREVLNDT | |
| | ISYPDGTFMDWEFKISVLYDIAKGMSYLHSSK | |
| | TEVHGRLKSTNCVVDSRMVVKITDFGCNSILP | |
| | PKKDLWTAPEHLRQANVSQKGDVYSYGIIAQE | |
| | IILRKETFYTSSCRDRNEKIFRVENSNGMKPF | |
| | RPDLFLETAEEKELEVYLLVKSCWEEDPEKRP | |
| | DFKKIETTLAKIFGLFHDQKNESYMDTLIRRL | |
| | QLYSRNLEHLVEERTQLYKAERDRADRLNFML | |
| | LPRLVVKSLKEKGFVEPELYEEVTIYFSDIVG | |
| | FTTICKYSTPMEVVDMLNDIYKSFDHIVDHHD | |
| | VYKVETIGDAYMVASGLPKRNGNRHAIDIAKM | |
| | ALEILSFMGTFELEHLPGLPIWIRIGVHSGPC | |
| | AAGVVGIKMPRYCLFGDTVNTASRMESTGLPL | |
| | RIHVSGSTIAILKRTECQFLYEVRGETYLKGR | |
| | GNETTYWLTGMKDQKFNLPTPPTVENQQRLQA | |
| | EFSDMIANSLQKRQAAGIRSQKPRRVASYKKG | |
| | TLEYLQLNTTDKESTYF | |
| 78 | DIQMTQSPSSLSASAGDRVTITCRASQSISSY | mAb1 light chain (LC) |
| | LNWYQQKPGKAPKLLIYAASSLQSGVPSRFSG | |
| | SGSGTDFTLAISSLOPEDFATYYCQQSYSTPW | |
| | TFGQGTKVDIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| 79 | QVQLVQSGAEVKKPGASVKVSCKASGYIFISS | mAb1 heavy chain (HC) |
| | AMHWVRQAPGQRLEWMGLINPGNGNTKYSQKF | comprising the mutation |
| | QGRVTITRDTSASTAYMELSSLRSEDTAVYYC | D265C (according to EU |
| | ARAYFRGLGFDIWGQGTLVTVSSASTKGPSVF | numbering system) |
| | PLAPSSKSTSGGTAALGCLVKDYFPEPVTVSW | |
| | NSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 80 | QVQLVQSGAEVKKPGASVKVSCKASGYIFISS | mAb1 heavy chain (HC) |
| | AMHWVRQAPGQRLEWMGLINPGNGNTKYSQKF | comprising mAb1 heavy |
| | QGRVTITRDTSASTAYMELSSLRSEDTAVYYC | chain (HC), comprising |
| | ARAYFRGLGFDIWGQGTLVTVSSASTKGPSVF | the mutations L234A, |
| | PLAPSSKSTSGGTAALGCLVKDYFPEPVTVSW | L235A and D265C |
| | NSGALTSGVHTFPAVLQSSGLYSLSSVVTVPS | (numbering according to |
| | SSLGTQTYICNVNHKPSNTKVDKKVEPKSCDK | EU numbering system) |
| | THTCPPCPAPEAAGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 81 | QVQLVQSGAEVKKPGASVKVSCKASGYIFISS | mAb1 heavy chain (HC), |
| | AMHWVRQAPGQRLEWMGLINPGNGNTKYSQKF | comprising the mutations |
| | QGRVTITRDTSASTAYMELSSLRSEDTAVYYC | L234A, L235A, G236R |
| | ARAYFRGLGFDIWGQGTLVTVSSASTKGPSVF | and D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEAARGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 82 | QVQLVQSGAEVKKPGASVKVSCKASGYIFISS | mAb1 heavy chain (HC), |
| | AMHWVRQAPGQRLEWMGLINPGNGNTKYSQKF | comprising the mutations |
| | QGRVTITRDTSASTAYMELSSLRSEDTAVYYC | L234G, L235S, G236R |
| | ARAYFRGLGFDIWGQGTLVTVSSASTKGPSVF | and D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEGSRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 83 | QVQLVQSGAEVKKPGASVKVSCKASGYIFISS | mAb1 heavy chain (HC) |
| | AMHWVRQAPGQRLEWMGLINPGNGNTKYSQKF | comprising the mutations |
| | QGRVTITRDTSASTAYMELSSLRSEDTAVYYC | L234S, L235T, G236R |
| | ARAYFRGLGFDIWGQGTLVTVSSASTKGPSVF | and D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESTRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 84 | QVQLVQSGAEVKKPGASVKVSCKASGYIFISS | mAb1 heavy chain (HC) |
| | AMHWVRQAPGQRLEWMGLINPGNGNTKYSQKF | comprising the mutations |
| | QGRVTITRDTSASTAYMELSSLRSEDTAVYYC | L234S, L235G, G236R |
| | ARAYFRGLGFDIWGQGTLVTVSSASTKGPSVF | and D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESGRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 85 | DIRMTQSPSSLSASAGDRVTITCRASQSISSY | mAb8 LC |
| | LNWYQQKPGKAPKLLIYAASSLQSGVPSRFSG | |
| | SGSGTDFTLTISSLQPEDFATYYCQQSYSTPL | |
| | TFGGGTKLEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| 86 | QVQLVQSGAEVKKPGASVKVSCKASGYIFISS | mAb8 HC comprising the |
| | AMHWVRQAPGQRLEWMGLINPGNGNTKYSQKF | mutation D265C |
| | QGRVTITRDTSASTAYMELSSLRSEDTAVYYC | (according to EU |
| | ARAYFRGLGFDIWGQGTLVTVSSASTKGPSVF | numbering system) |
| | PLAPSSKSTSGGTAALGCLVKDYFPEPVTVSW | |
| | NSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 87 | QVQLVQSGAEVKKPGASVKVSCKASGYIFISS | mAb8 HC comprising the |
| | AMHWVRQAPGQRLEWMGLINPGNGNTKYSQKF | mutations L234A, L235A |
| | QGRVTITRDTSASTAYMELSSLRSEDTAVYYC | and D265C (numbering |
| | ARAYFRGLGFDIWGQGTLVTVSSASTKGPSVF | according to EU |
| | PLAPSSKSTSGGTAALGCLVKDYFPEPVTVSW | numbering system) |
| | NSGALTSGVHTFPAVLQSSGLYSLSSVVTVPS | |
| | SSLGTQTYICNVNHKPSNTKVDKKVEPKSCDK | |
| | THTCPPCPAPEAAGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 88 | QVQLVQSGAEVKKPGASVKVSCKASGYIFISS | mAb8 HC comprising the |
| | AMHWVRQAPGQRLEWMGLINPGNGNTKYSQKF | mutations L234A, L235A, |
| | QGRVTITRDTSASTAYMELSSLRSEDTAVYYC | G236R and D265C |
| | ARAYFRGLGFDIWGQGTLVTVSSASTKGPSVF | (numbering according to |
| | PLAPSSKSTSGGTAALGCLVKDYFPEPVTVSW | EU numbering system) |
| | NSGALTSGVHTFPAVLQSSGLYSLSSVVTVPS | |
| | SSLGTQTYICNVNHKPSNTKVDKKVEPKSCDK | |
| | THTCPPCPAPEAARGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 89 | QVQLVQSGAEVKKPGASVKVSCKASGYIFISS | mAb8 HC comprising the |
| | AMHWVRQAPGQRLEWMGLINPGNGNTKYSQKF | mutations L234G, |
| | QGRVTITRDTSASTAYMELSSLRSEDTAVYYC | L235S, G236R and |
| | ARAYFRGLGFDIWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEGSRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 90 | QVQLVQSGAEVKKPGASVKVSCKASGYIFISS | mAb8 HC comprising the |
| | AMHWVRQAPGQRLEWMGLINPGNGNTKYSQKF | mutations L234S, L235T, |
| | QGRVTITRDTSASTAYMELSSLRSEDTAVYYC | G236R and D265C |
| | ARAYFRGLGFDIWGQGTLVTVSSASTKGPSVF | (numbering according to |
| | PLAPSSKSTSGGTAALGCLVKDYFPEPVTVSW | EU numbering system) |
| | NSGALTSGVHTFPAVLQSSGLYSLSSVVTVPS | |
| | SSLGTQTYICNVNHKPSNTKVDKKVEPKSCDK | |
| | THTCPPCPAPESTRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 91 | QVQLVQSGAEVKKPGASVKVSCKASGYIFISS | mAb8 HC comprising the |
| | AMHWVRQAPGQRLEWMGLINPGNGNTKYSQKF | mutations L234S, |
| | QGRVTITRDTSASTAYMELSSLRSEDTAVYYC | L235G, G236R and |
| | ARAYFRGLGFDIWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESGRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 92 | DIQMTQSPSSLSASAGDRVTITCRASQSISSY | mAb24 LC |
| | LNWYQQKPGKAPKLLIYAASSLQSGVPSRFSG | |
| | SGSGTDFTLAISSLQPEDFATYYCQQSYSTPY | |
| | TFGQGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| 93 | QVQLVQSGAEVKKPGASVKVSCKASGYIFISS | mAb24 HC comprising |
| | AMHWVRQAPGQRLEWMGLINPGNGNTKYSQKF | the mutation D265C |
| | QGRVTITRDTSASTAYMELSSLRSEDTAVYYC | (numbering according to |
| | ARAYFRGLGFDIWGQGTLVTVSSASTKGPSVF | EU numbering system) |
| | PLAPSSKSTSGGTAALGCLVKDYFPEPVTVSW | |
| | NSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 94 | QVQLVQSGAEVKKPGASVKVSCKASGYIFISS | mAb24 HC comprising |
| | AMHWVRQAPGQRLEWMGLINPGNGNTKYSQKF | the mutations L234A, |
| | QGRVTITRDTSASTAYMELSSLRSEDTAVYYC | L235A and D265C |
| | ARAYFRGLGFDIWGQGTLVTVSSASTKGPSVF | (numbering according to |
| | PLAPSSKSTSGGTAALGCLVKDYFPEPVTVSW | EU numbering system) |
| | NSGALTSGVHTFPAVLQSSGLYSLSSVVTVPS | |
| | SSLGTQTYICNVNHKPSNTKVDKKVEPKSCDK | |
| | THTCPPCPAPEAAGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 95 | QVQLVQSGAEVKKPGASVKVSCKASGYIFISS | mAb24 heavy chain |
| | AMHWVRQAPGQRLEWMGLINPGNGNTKYSQKF | (HC), comprising the |
| | QGRVTITRDTSASTAYMELSSLRSEDTAVYYC | mutations L234A, L235A, |
| | ARAYFRGLGFDIWGQGTLVTVSSASTKGPSVF | G236R and D265C |
| | PLAPSSKSTSGGTAALGCLVKDYFPEPVTVSW | (numbering according to |
| | NSGALTSGVHTFPAVLQSSGLYSLSSVVTVPS | EU numbering system) |
| | SSLGTQTYICNVNHKPSNTKVDKKVEPKSCDK | |
| | THTCPPCPAPEAARGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 96 | QVQLVQSGAEVKKPGASVKVSCKASGYIFISS | mAb24 heavy chain (HC) |
| | AMHWVRQAPGQRLEWMGLINPGNGNTKYSQKF | comprising the mutations |
| | QGRVTITRDTSASTAYMELSSLRSEDTAVYYC | L234G, L235S, G236R |
| | ARAYFRGLGFDIWGQGTLVTVSSASTKGPSVF | and D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEGSRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 97 | QVQLVQSGAEVKKPGASVKVSCKASGYIFISS | mAb24 HC comprising |
| | AMHWVRQAPGQRLEWMGLINPGNGNTKYSQKF | the mutations L234S, |
| | QGRVTITRDTSASTAYMELSSLRSEDTAVYYC | L235T, G236R and |
| | ARAYFRGLGFDIWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESTRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 98 | QVQLVQSGAEVKKPGASVKVSCKASGYIFISS | mAb24 HC comprising |
| | AMHWVRQAPGQRLEWMGLINPGNGNTKYSQKF | the mutations L234S, |
| | QGRVTITRDTSASTAYMELSSLRSEDTAVYYC | L235G, G236R and |
| | ARAYFRGLGFDIWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESGRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 99 | QSGLTQPRSVSGSPGQSVTISCTGTSSDVGGY | mAb28 LC |
| | KYVSWYQQHPGKAPKLKIYDVSKRPSGVPDRF | |
| | SGSKSGNTASLTISGLQAEDEADYYCTSYTSS | |
| | NTVVFGGGTKVTVLGQPKAAPSVTLFPPSSEE | |
| | LQANKATLVCLISDFYPGAVTVAWKADSSPVK | |
| | AGVETTTPSKQSNNKYAASSYLSLTPEQWKSH | |
| | RSYSCQVTHEGSTVEKTVAPTECS | |
| 100 | QVQLVESGAEVKKPGASVKVSCKASGYTFTSY | mAb28 HC comprising |
| | AISWVRQAPGQGLEWMGRIIPILGIANYAQKF | the mutation D265C |
| | QGRVTITADKSTSTAYMELSRLRSDDTAVYYC | (numbering according to |
| | AIDSSGWMTFDYWGQGTMVTVSSASTKGPSVF | EU numbering system) |
| | PLAPSSKSTSGGTAALGCLVKDYFPEPVTVSW | |
| | NSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 101 | QVQLVESGAEVKKPGASVKVSCKASGYTFTSY | mAb28 HC comprising |
| | AISWVRQAPGQGLEWMGRIIPILGIANYAQKF | the mutations L234A, |
| | QGRVTITADKSTSTAYMELSRLRSDDTAVYYC | L235A and D265C |
| | AIDSSGWMTFDYWGQGTMVTVSSASTKGPSVF | (numbering according to |
| | PLAPSSKSTSGGTAALGCLVKDYFPEPVTVSW | EU numbering system) |
| | NSGALTSGVHTFPAVLQSSGLYSLSSVVTVPS | |
| | SSLGTQTYICNVNHKPSNTKVDKKVEPKSCDK | |
| | THTCPPCPAPEAAGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 102 | QVQLVESGAEVKKPGASVKVSCKASGYTFTSY | mAb28 HC comprising |
| | AISWVRQAPGQGLEWMGRIIPILGIANYAQKF | the mutations L234A, |
| | QGRVTITADKSTSTAYMELSRLRSDDTAVYYC | L235A, G236R and |
| | AIDSSGWMTFDYWGQGTMVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEAARGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 103 | QVQLVESGAEVKKPGASVKVSCKASGYTFTSY | mAb28 HC comprising |
| | AISWVRQAPGQGLEWMGRIIPILGIANYAQKF | the mutations L234G, |
| | QGRVTITADKSTSTAYMELSRLRSDDTAVYYC | L235S, G236R and |
| | AIDSSGWMTFDYWGQGTMVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEGSRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 104 | QVQLVESGAEVKKPGASVKVSCKASGYTFTSY | mAb28 HC comprising |
| | AISWVRQAPGQGLEWMGRIIPILGIANYAQKF | the mutations L234S, |
| | QGRVTITADKSTSTAYMELSRLRSDDTAVYYC | L235T, G236R and |
| | AIDSSGWMTFDYWGQGTMVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESTRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 105 | QVQLVESGAEVKKPGASVKVSCKASGYTFTSY | mAb28 HC comprising |
| | AISWVRQAPGQGLEWMGRIIPILGIANYAQKF | the mutations L234S, |
| | QGRVTITADKSTSTAYMELSRLRSDDTAVYYC | L235G, G236R and |
| | AIDSSGWMTFDYWGQGTMVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESGRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 106 | QSVLTQPPSASGTPGQRVTISCSGSSSNIGSN | mAb40 LC |
| | TVNWYQQLPGTAPRLLIYSNRQRPSGVPDRFS | |
| | GARSGTSASLAITGLQPEDEADYYCATWDVGL | |
| | RGRVFGGGTKLTVLGQPKAAPSVTLFPPSSEE | |
| | LQANKATLVCLISDFYPGAVTVAWKADSSPVK | |
| | AGVETTTPSKQSNNKYAASSYLSLTPEQWKSH | |
| | RSYSCQVTHEGSTVEKTVAPTECS | |
| 107 | EVQLVQSGAEVKKPGASVKVSCKASGYTFTSY | mAb40 HC comprising |
| | YMHWVRQAPGQGLEWMGIINPSGGSTSYAQKF | the mutation D265C |
| | QGRVTMTRDTSTSTVYMELSSLRSEDTAVYYC | (numbering according to |
| | ARVRQWPLASDYWGQGTLVTVSSASTKGPSVF | EU numbering system) |
| | PLAPSSKSTSGGTAALGCLVKDYFPEPVTVSW | |
| | NSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 108 | EVQLVQSGAEVKKPGASVKVSCKASGYTFTSY | mAb40 HC comprising |
| | YMHWVRQAPGQGLEWMGIINPSGGSTSYAQKF | the mutations L234A, |
| | QGRVTMTRDTSTSTVYMELSSLRSEDTAVYYC | L235A and D265C |
| | ARVRQWPLASDYWGQGTLVTVSSASTKGPSVF | (numbering according to |
| | PLAPSSKSTSGGTAALGCLVKDYFPEPVTVSW | EU numbering system) |
| | NSGALTSGVHTFPAVLQSSGLYSLSSVVTVPS | |
| | SSLGTQTYICNVNHKPSNTKVDKKVEPKSCDK | |
| | THTCPPCPAPEAAGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 109 | EVQLVQSGAEVKKPGASVKVSCKASGYTFTSY | mAb40 HC comprising |
| | YMHWVRQAPGQGLEWMGIINPSGGSTSYAQKF | the mutations L234A, |
| | QGRVTMTRDTSTSTVYMELSSLRSEDTAVYYC | L235A, G236R and |
| | ARVRQWPLASDYWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEAARGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 110 | EVQLVQSGAEVKKPGASVKVSCKASGYTFTSY | mAb40 HC comprising |
| | YMHWVRQAPGQGLEWMGIINPSGGSTSYAQKF | the mutations L234G, |
| | QGRVTMTRDTSTSTVYMELSSLRSEDTAVYYC | L235S, G236R and |
| | ARVRQWPLASDYWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPEGSRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 111 | EVQLVQSGAEVKKPGASVKVSCKASGYTFTSY | mAb40 HC comprising |
| | YMHWVRQAPGQGLEWMGIINPSGGSTSYAQKF | the mutations L234S, |
| | QGRVTMTRDTSTSTVYMELSSLRSEDTAVYYC | L235T, G236R and |
| | ARVRQWPLASDYWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESTRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 112 | EVQLVQSGAEVKKPGASVKVSCKASGYTFTSY | mAb40 HC comprising |
| | YMHWVRQAPGQGLEWMGIINPSGGSTSYAQKF | the mutations L234S, |
| | QGRVTMTRDTSTSTVYMELSSLRSEDTAVYYC | L235G, G236R and |
| | ARVRQWPLASDYWGQGTLVTVSSASTKGPSVF | D265C (numbering |
| | PLAPSSKSTSGGTAALGCLVKDYFPEPVTVSW | according to EU |
| | NSGALTSGVHTFPAVLQSSGLYSLSSVVTVPS | numbering system) |
| | SSLGTQTYICNVNHKPSNTKVDKKVEPKSCDK | |
| | THTCPPCPAPESGRGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 113 | QSALTQPASVSGSPGQSITISCTGTSSDVGSY | mAb41 LC |
| | NLVSWYQQHPGKAPKLMIYEGSKRPSGVSNRF | |
| | SASKSGNTASLTISGLQTEDEADYYCSSYVPR | |
| | SSLVFGGGTKLTVLGQPKAAPSVTLFPPSSEE | |
| | LQANKATLVCLISDFYPGAVTVAWKADSSPVK | |
| | AGVETTTPSKQSNNKYAASSYLSLTPEQWKSH | |
| | RSYSCQVTHEGSTVEKTVAPTECS | |
| 114 | QVQLVQSGAEVKKPGSSVKVSCKASGGTFSTY | mAb41 HC comprising |
| | TINWVRQAPGQGLEWMGRIIPVLGIANYAQKF | the mutation D265C |
| | QGRVTITADKSTSTAYMELSSLRSEDTAVYYC | (numbering according to |
| | ARDTPRLRSSYYMDVWGQGTLVTVSSASTKGP | EU numbering system) |
| | SVFPLAPSSKSTSGGTAALGCLVKDYFPEPVT | |
| | VSWNSGALTSGVHTFPAVLQSSGLYSLSSVVT | |
| | VPSSSLGTQTYICNVNHKPSNTKVDKKVEPKS | |
| | CDKTHTCPPCPAPELLGGPSVFLFPPKPKDTL | |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |
| 115 | QVQLVQSGAEVKKPGSSVKVSCKASGGTFSTY | mAb41 HC comprising |
| | TINWVRQAPGQGLEWMGRIIPVLGIANYAQKF | the mutations L234A, |
| | QGRVTITADKSTSTAYMELSSLRSEDTAVYYC | L235A and D265C |
| | ARDTPRLRSSYYMDVWGQGTLVTVSSASTKGP | (numbering according to |
| | SVFPLAPSSKSTSGGTAALGCLVKDYFPEPVT | EU numbering system) |
| | VSWNSGALTSGVHTFPAVLQSSGLYSLSSVVT | |
| | VPSSSLGTQTYICNVNHKPSNTKVDKKVEPKS | |
| | CDKTHTCPPCPAPEAAGGPSVFLFPPKPKDTL | |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |
| 116 | QVQLVQSGAEVKKPGSSVKVSCKASGGTFSTY | mAb41 HC comprising |
| | TINWVRQAPGQGLEWMGRIIPVLGIANYAQKF | the mutations L234A, |
| | QGRVTITADKSTSTAYMELSSLRSEDTAVYYC | L235A, G236R and |
| | ARDTPRLRSSYYMDVWGQGTLVTVSSASTKGP | D265C (numbering |
| | SVFPLAPSSKSTSGGTAALGCLVKDYFPEPVT | according to EU |
| | VSWNSGALTSGVHTFPAVLQSSGLYSLSSVVT | numbering system) |
| | VPSSSLGTQTYICNVNHKPSNTKVDKKVEPKS | |
| | CDKTHTCPPCPAPEAARGPSVFLFPPKPKDTL | |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |
| 117 | QVQLVQSGAEVKKPGSSVKVSCKASGGTFSTY | mAb41 HC comprising |
| | TINWVRQAPGQGLEWMGRIIPVLGIANYAQKF | the mutations L234G, |
| | QGRVTITADKSTSTAYMELSSLRSEDTAVYYC | L235S, G236R and |
| | ARDTPRLRSSYYMDVWGQGTLVTVSSASTKGP | D265C (numbering |
| | SVFPLAPSSKSTSGGTAALGCLVKDYFPEPVT | according to EU |
| | VSWNSGALTSGVHTFPAVLQSSGLYSLSSVVT | numbering system) |
| | VPSSSLGTQTYICNVNHKPSNTKVDKKVEPKS | |
| | CDKTHTCPPCPAPEGSRGPSVFLFPPKPKDTL | |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |
| 118 | QVQLVQSGAEVKKPGSSVKVSCKASGGTFSTY | mAb41 HC comprising |
| | TINWVRQAPGQGLEWMGRIIPVLGIANYAQKF | the mutations L234S, |
| | QGRVTITADKSTSTAYMELSSLRSEDTAVYYC | L235T, G236R and |
| | ARDTPRLRSSYYMDVWGQGTLVTVSSASTKGP | D265C (numbering |
| | SVFPLAPSSKSTSGGTAALGCLVKDYFPEPVT | according to EU |
| | VSWNSGALTSGVHTFPAVLQSSGLYSLSSVVT | numbering system) |
| | VPSSSLGTQTYICNVNHKPSNTKVDKKVEPKS | |
| | CDKTHTCPPCPAPESTRGPSVFLFPPKPKDTL | |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |
| 119 | QVQLVQSGAEVKKPGSSVKVSCKASGGTFSTY | mAb41 HC comprising |
| | TINWVRQAPGQGLEWMGRIIPVLGIANYAQKF | the mutations L234S, |
| | QGRVTITADKSTSTAYMELSSLRSEDTAVYYC | L235G, G236R and |
| | ARDTPRLRSSYYMDVWGQGTLVTVSSASTKGP | D265C (numbering |
| | SVFPLAPSSKSTSGGTAALGCLVKDYFPEPVT | according to EU |
| | VSWNSGALTSGVHTFPAVLQSSGLYSLSSVVT | numbering system) |
| | VPSSSLGTQTYICNVNHKPSNTKVDKKVEPKS | |
| | CDKTHTCPPCPAPESGRGPSVFLFPPKPKDTL | |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |
| 120 | RIIPVLGIANYAQKFQG | mAb 41 HC CDR2 |
| 121 | DIQMTQSPSSLSASAGDRVTITO | mAb1, 24 LC FR1 |
| 122 | CDPIWGIG | alpha-amanitin, with |
| | | Hydroxy-proline (aa3), |
| | | and Dihydroxyisoleucine |
| | | (aa4) |

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1: Synthesis of Amatoxins

Various amanitin derivatives were used for conjugation with the antibody of the invention. The amanitin derivatives were generated according to the methods as disclosed in WO2016/142049, WNO2017/149077, WNO2018/115466, WNO2019/197654, WNO2019/030173, WO20216947A1, and WO2022/096604 the corresponding disclosures of which are incorporated herein in its entirety. Amanitin derivative (IX) of the invention can be obtained by in analogous fashion to the synthesis of compound XVIa as disclosed in WO2022/096604 using beta-amanitin.

### Synthesis of Amatoxin-Linker Payload for Conjugate (XXII)

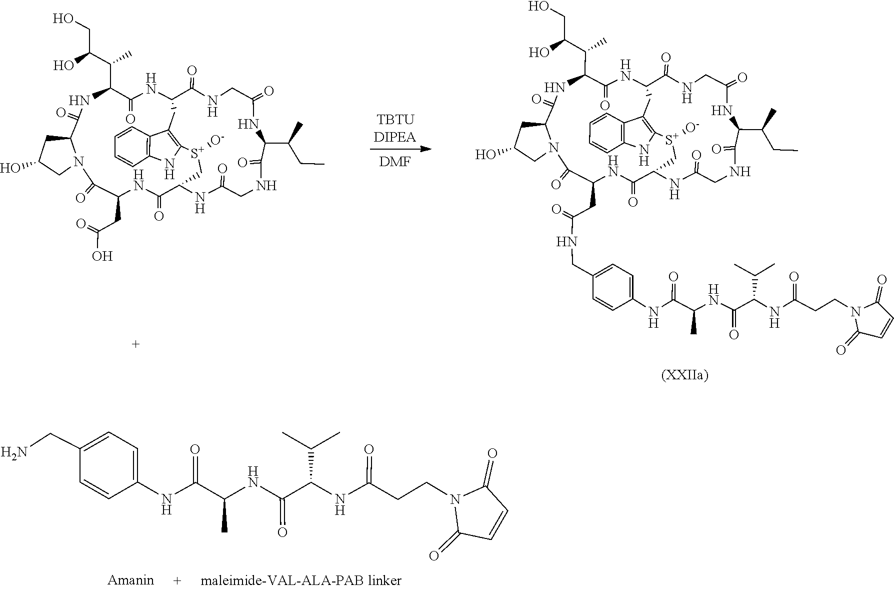

Natural amanin (9.04 mg, 10 μmol) and maleimide-VAL-ALA-PAB linker (13.3 mg, 3 eq.) was dissolved in 300 μl DMF. A 0.1 M solution of TBTU in DMF (30 μmol, 3 eq) and 300 μl of 0.2 M DIPEA in DMF (60 μmol, 6.0 eq) at RT were added. The reaction was monitored by RP-HPLC. After 2 h the reaction was quenched with 100 μl water and the crude was purified by preparative RP-HPLC. Product containing fractions were evaporated and freeze-dried from 2 ml tert-butanol/water 4:1:

Yield: 5.39 mg (41%) colourless lyophilisate

MS (ESI+) [MH]⁺ found: 1329.85; calc.: 1329.56 (C₆₁H₈₁N₁₄O₁₈S⁺)

The resulting amatoxin-linker conjugate (XXIIa) can be reacted as disclosed in Example 2 below to yield conjugate (XXII) of the invention.

### Example 2: Synthesis of Amatoxin-Linker Constructs

Antibodies were conjugated to the amatoxin linker conjugates by means of the so-called Thiomab technology. In this approach, the conjugation takes place by coupling of the maleimide residue of the toxin linker construct to the free SH group of a cysteine residue in the antibody, as shown in the following reaction scheme:

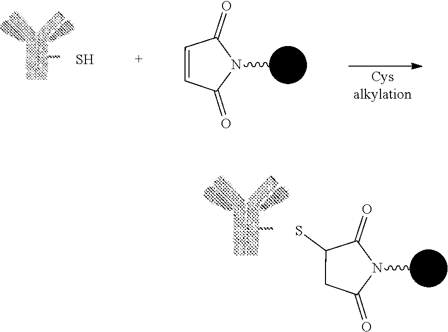

The principles of this conjugation method are disclosed in Junutula et al. (2008), the content of which is incorporated herein by reference.

The antibodies used in the present experiments comprise a D265C substitution in both Fc domains, in order to provide a cystein residue that has a free SH group. The respective technology is disclosed in WO2016/142049 A1, the content of which is incorporated herein by reference, and which results in a homogenous product with a fixed drug to antibody ratio (“DAR”) of about 2 and a site specific conjugation.

### Example 3: In Vitro Cytotoxicity of Anti-GUCY2C Conjugates of the Invention

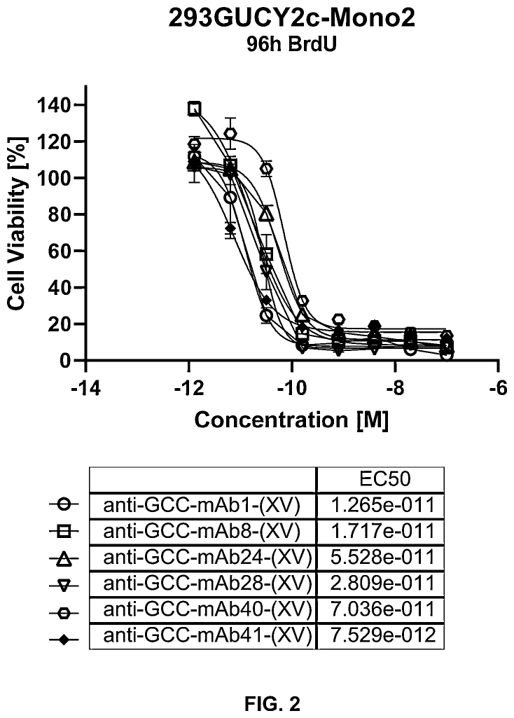

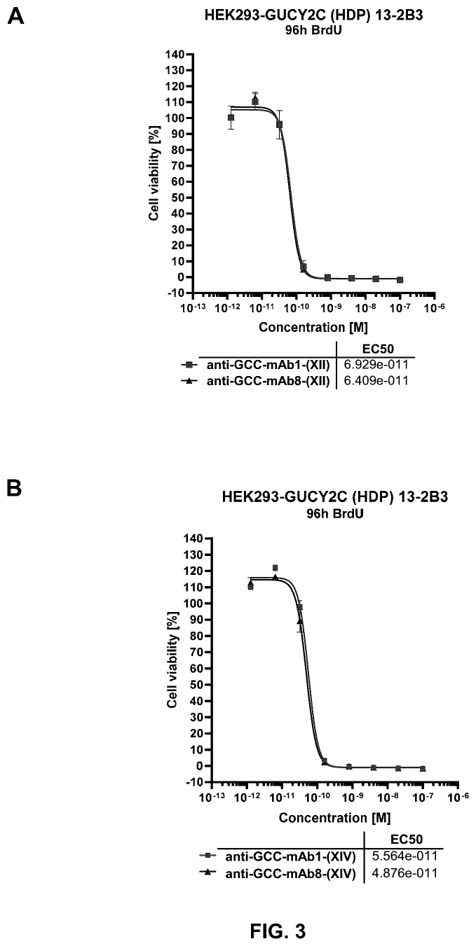

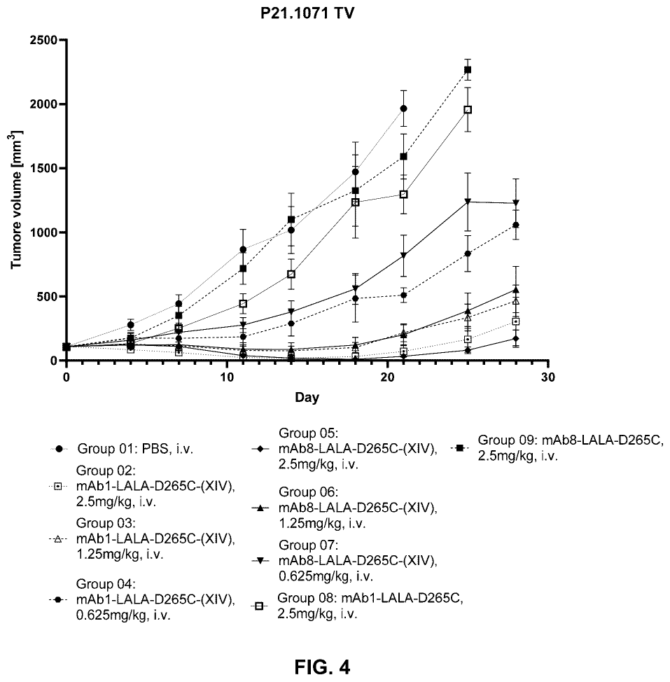

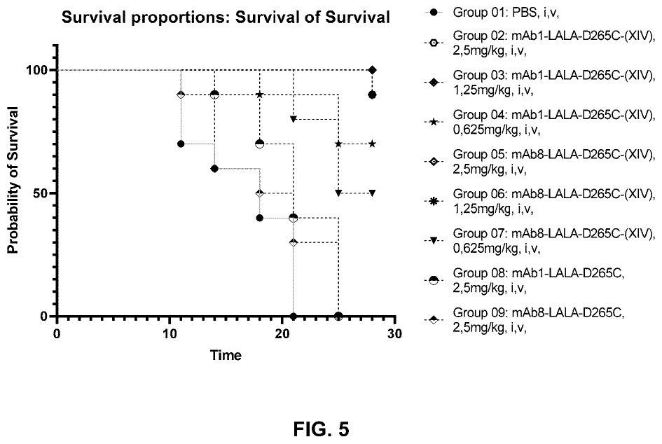

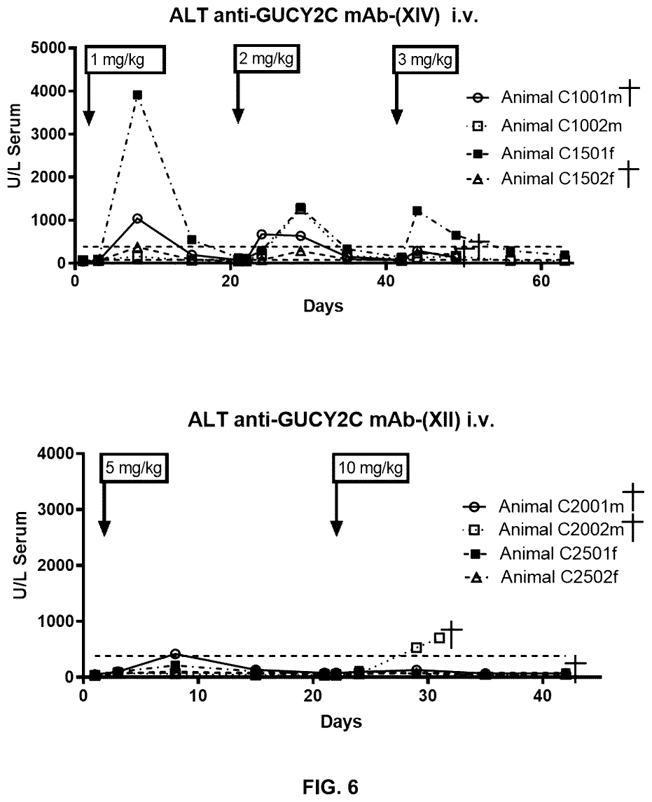

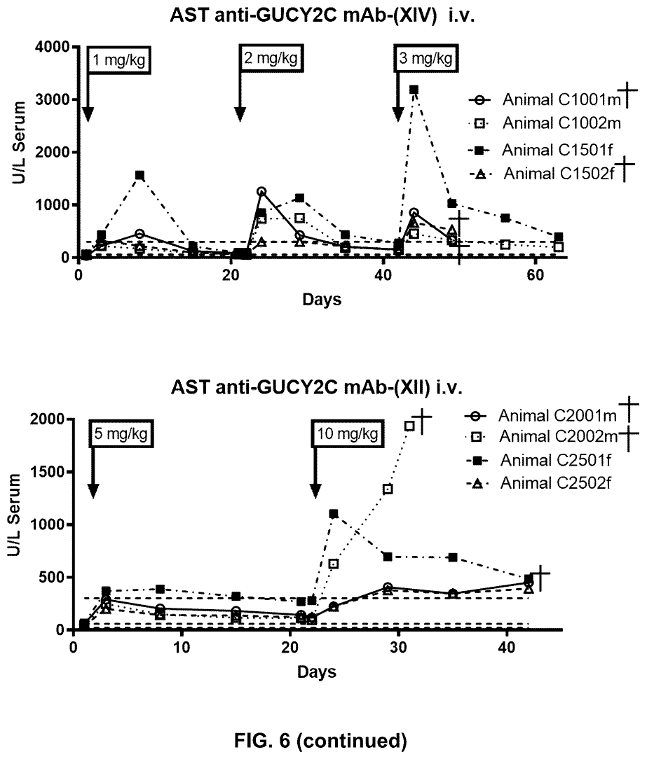

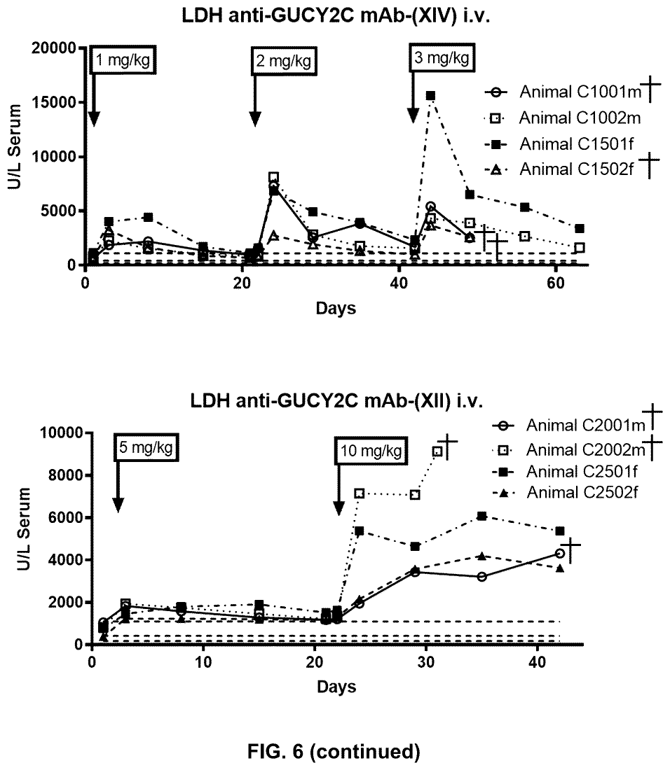

In vitro cytotoxicity studies as depicted in FIG. 2 and FIG. 3 were performed on human GUCY2C-positive HEK293-GUCY2C-Mono2 cells (Creative Biogen), or on HEK293-GUCY2C-HDP-2B3 cells overexpressing human GUCY2C. The quantitative determination of cell viability was done using a commercially available BrdU kit 96 hours post conjugate addition to the growth medium of the cells.

HEK293-GUCY2C-HDP-2B3 which express human GUCY2C on the cell surface were obtained by transient transfection of HEK293 wild-type cells with an overexpression plasmid encoding human guanylate cyclase 2C (GUCY2C or GCC, Uniprot no. P25092) having the amino acid sequence according to SEQ ID NO: 76 and a resistance against geneticin (G418). Selection of stably transfected cells was done after 4 days by placing the cells in culture medium containing G418 to generate a stable cell pool expressing GCC and G418 resistance. A stable single-cell clone was isolated from this cell pool by using limiting dilution. GCC surface expression was confirmed for the stable cell clone HEK293-GUCY2C-HDP-2B3 by flow cytometry and cytotoxicity assay (BrdU ELISA), respectively.

### Example 4: Efficacy of the Anti-GUCY2C Conjugates in a Subcutaneous Human Embryonic Kidney Model after Single Dose Intravenous Application

In this study 9 experimental groups with 8-10 animals each (Female NOD SCID mice, n=126, age at start of experiment 7 weeks) bearing subcutaneous HEK293-GUCY2C-(HDP)-2B3 tumors were used (see Table 1). On day −16 (16 days prior to the start of the experiment), 126 mice were inoculated subcutaneously with 5×10⁶ HEK293-GUCY2C-(HDP)-2B3 tumor cells in 200 μL RPMI w/o phenol red containing 50% Matrigel@ into their right flanks. Once a mean tumor volume of approximately 150 mm³ was reached, animals were randomized into 9 groups according to tumor sizes Therapy was initiated one day after group allocation. Mice were treated with PBS, mAb1-LALA-D265C-(XIV), mAb8-LALA-D265C-(XIV), mAb1-LALA-D265C or mAb8-LALA-D265C (for details see Table 1). Tumor volumes were measured twice per week by calliper (see below) and body weight was determined in parallel. Animals were sacrificed, and a necropsy performed, if either tumor volumes were >1600 mm³ or mice needed to be sacrificed due to ethical reasons.

Tumor growth was monitored by calliper measurement. Tumor size was calculated according to the formula W²×L/2 (L=length and W=perpendicular width of the tumor, L>W). If unusual growth was observed then tumor volume was calculated according the formula: [(L×W×D))/6000]×U.

Both conjugates mAb1-LALA-D265C-(XIV) and mAb8-LALA-D265C-(XIV) of the invention had a DAR of 2.

TABLE 1
Experimental groups

| | | dose | | | |
|---|---|---|---|---|---|
| | | protein | | | Animals |
| Group | Compound | [mg/kg] | Schedule | Route | (n) |
| 1 | PBS | 10 mL/kg | 1x | i.v. | 8-10 |
| 2 | mAb1-LALA-D265C- | 2.5 | 1x | i.v. | 8-10 |
| | (XIV) | | | | |
| 3 | mAb1-LALA-D265C- | 1.25 | 1x | i.v. | 8-10 |
| | (XIV) | | | | |
| 4 | mAb1-LALA-D265C- | 0.625 | 1x | i.v. | 8-10 |
| | (XIV) | | | | |
| 5 | mAb8-LALA-D265C- | 2.5 | 1x | i.v. | 8-10 |
| | (XIV) | | | | |
| 6 | mAb8-LALA-D265C- | 1.25 | 1x | i.v. | 8-10 |
| | (XIV) | | | | |
| 7 | mAb8-LALA-D265C- | 0.625 | 1x | i.v. | 8-10 |
| | (XIV) | | | | |
| 8 | mAb1-LALA-D265C | 2.5 | 1x | i.v. | 8-10 |
| 9 | mAb8-LALA-D265C | 2.5 | 1x | i.v. | 8-10 |

“mAb1-LALA-D265C”, “mAb8-LALA-D265C” refer to antibodies comprising heavy and light chain amino acid sequences SEQ ID NO: 78, SEQ ID NO: 80, and SEQ ID NO: 85, SEQ ID NO: 87, respectively.

### Example 5: Toxicity and Serum Pharmacokinetics of the Conjugates of the Invention in Cynomolgus Monkeys

The purpose of this study was to determine the toxicity and serum pharmacokinetics of conjugates of the invention, mAb8-LALA-D265C-(XIV) and, mAb8-LALA-D265C-(XII) following a single intravenous (i.v.) infusion administration to male and female naïve cynomolgus monkeys.

### Study Design

Eight (8, 4/sex) naïve cynomolgus monkeys were divided into two groups with 4 animals/group, Group 1 were included three phases, Group 2 were included two phases. Animals in Group 1, Phases 1, 2 and 3 (Days 1, 22 and 43) were administered with mAb8-LALA-D265C-(XIV) by single intravenous infusion administration at 1, 2 and 3 mg/kg, respectively. Animals in Group 2, Phases 1 and 2 (Days 1 and 22) were administered with mAb8-LALA-D265C-(XII) by single intravenous infusion administration at 5 and 10 mg/kg, respectively. Blood/serum samples were taken pre-dose, on day 3, 8, 15 and 22 after each dosing to determine the liver enzymes levels of ALT, AST and LDH in the serum.

### Dose Administration of Conjugates

Animals were weighed prior to dose administration on each day of dosing to calculate the actual dose volume. All animals in Groups 1 and 2 received a single intravenous infusion administration of mAb8-LALA-D265C-(XIV) and mAb8-LALA-D265C-(XII) from the peripheral vein.

### Blood Collection and Plasma Preparation

Approximately 0.8 mL blood was collected at each time point via peripheral vessel from each study animal. The actual time for each sample collection was recorded. The deviations on sampling time were less than 1 minute for the time points pre-dose through 1 hour post-dose, and less than 5% of the nominal time for time points after 1 hour post-dose.

All blood samples were collected into commercially available BD tubes containing polymer silica activator. After blood was collected, the tubes containing blood samples were rested at room temperature for at least 30 minutes. Then centrifugation at 4° C. for 10 minutes at 3200×g within 1 hour after collection. The clarified serum was then collected after centrifugation. Typically, the serum was divided to 2 aliquots one aliquot (about 150 μL) for PK analysis, and another aliquot for backup. Analysis of liver enzymes ALT, AST and LDH was done using commercial kits.

### Example 6: In Vivo Efficacy Study Evaluating Two Antibody-Drug Conjugates Colon Cancer PDX Models Grown Subcutaneously in NMRI Nude Mice

Patient derived xenografts (PDX) colon cancer models in NMRI nude mice were done at Charles River Discovery Research Services Germany GmbH in accordance with local animal welfare guidelines.

For the study 10 NMRI nude mice age 4-6 weeks per group were allocated, tumor volume at randomization was 50 to 250 mm³, preferably 80-200 mm². Tumor volume was assessed in mm³ by caliper twice a week

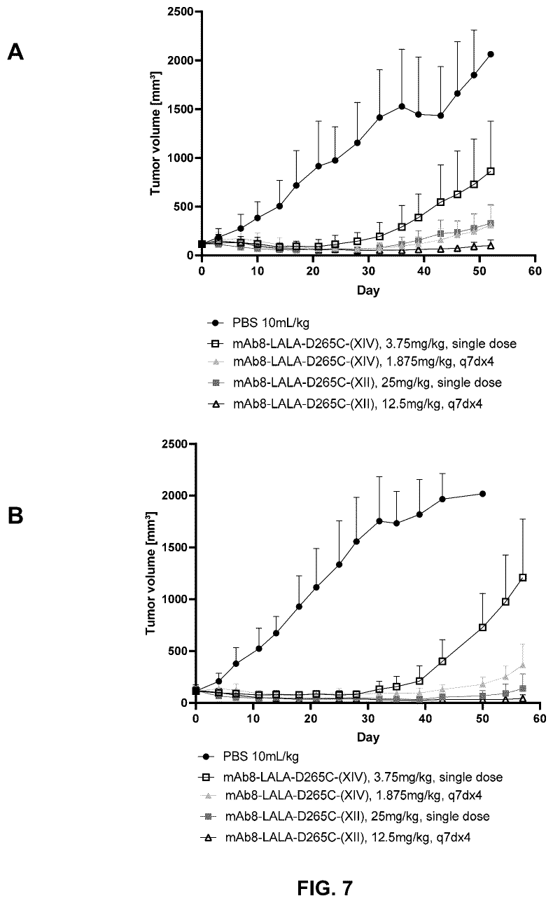

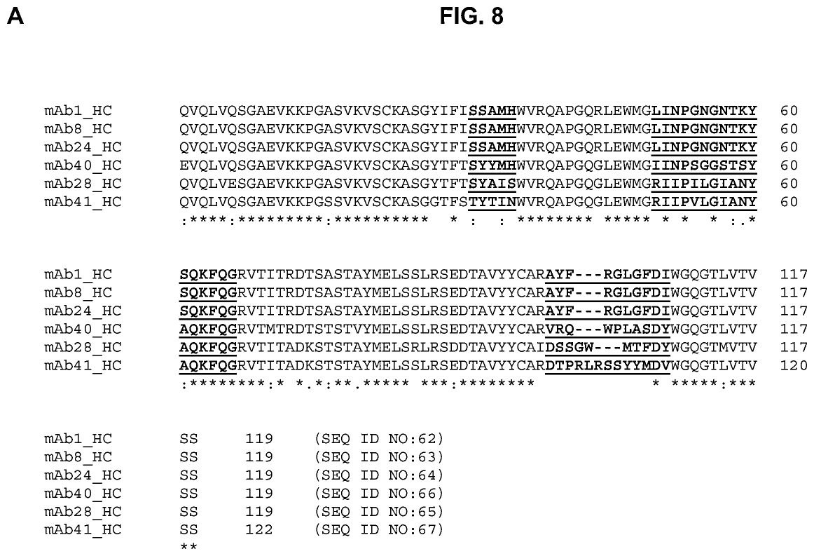

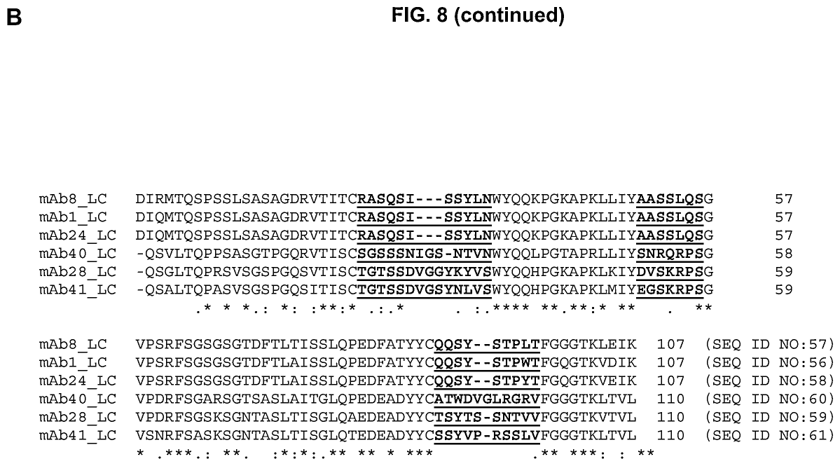

Conjugates of the invention, mAb8-LALA-D265C-(XII) and mAb8-LALA-D265C-(XIV) were dosed either as single dose (“single dose”, or once every 7 days for 4 weeks (“q7dx4”) as indicated in FIG. 7. Sequencing of tumor cDNA revealed a G13D mutation in codon 38 of KRAS in PDX model V887_CXF1034_TV (FIG. 7 (A)).

1. An isolated antibody or antibody fragment which specifically binds to guanylyl cyclase C (GUCY2C), wherein the antibody comprises a heavy chain variable region (V_{H}) which comprises a framework region 1 (FR_{H}1), complementarity-determining region 1 (CDR_{H}1), a FR_{H}2, a CDR_{H}2, a FR_{H}3, a CDR_{H}3, and a FR_{H}4, wherein
FR_{H}1 comprises an amino acid sequence selected from SEQ ID NOs: 33, 40, 45, and 51;
CDR_{H}1 comprises an amino acid sequence selected from SEQ ID NOs: 34, 41, 46, and 52;
FR_{H}2 comprises an amino acid sequence selected from SEQ ID NOs: 35 and 55;
CDR_{H}2 comprises an amino acid sequence selected from SEQ ID NOs: 36, 42, 47, and 120 FR_{H}3 comprises an amino acid sequence selected from SEQ ID NOs: 37, 43, 48, and 53;
CDR_{H}3 comprises an amino acid sequence selected from SEQ ID NOs: 38, 44, 49, and 54; and
FR_{H}4 comprises an amino acid sequence selected from SEQ ID NOs: 39 and 50.

2. The antibody or antibody fragment according to claim 1, wherein the antibody comprises a light chain variable region (V_{L}) which comprises a framework region 1 (FR_{L}1), complementarity-determining region (CDR_{L}1), a FR_{L}2, a CDR_{L}2, a FR_{L}3, a CDR_{L}3, and a FR_{L}4, wherein
FR_{L}1 comprises an amino acid sequence selected from SEQ ID NOs: 1, 13, 20, and 27;
CDR_{L}1 comprises an amino acid sequence selected from SEQ ID NOs: 2, 14, 21, and 28;
FR_{L}2 comprises an amino acid sequence selected from SEQ ID NOs: 3, 15, and 29
CDR_{L}2 comprises an amino acid sequence selected from SEQ ID NOs: 4, 16, 23, and 30;
FR_{L}3 comprises an amino acid sequence selected from SEQ ID NOs 5, 8, 17, 24, and 31;
CDR_{L}3 comprises an amino acid sequence selected from SEQ ID NOs: 6, 9, 11, 18, 25, and 32; and
FR_{L}4 comprises an amino acid sequence selected from SEQ ID NOs: 7, 10, 12, 19, and 26.

3. The antibody or antibody fragment according to claim 1, wherein the antibody or antibody fragment comprises a heavy chain variable region (V_{H}) which comprises a framework region 1 (FR_{H}1), complementarity-determining region 1 (CDR_{H}1), a FR_{H}2, a CDR_{H}2, a FR_{H}3, a CDR_{H}3, and a FR_{H}4, wherein
FR_{H}1 comprises an amino acid sequence according to SEQ ID NO: 33
CDR_{H}1 comprises an amino acid sequence according to SEQ ID NOs: 34,
FR_{H}2 comprises an amino acid sequence according to SEQ ID NO: 35;
CDR_{H}2 comprises an amino acid sequence according to SEQ ID NO: 36;
FR_{H}3 comprises an amino acid sequence according to SEQ ID NO: 37;
CDR_{H}3 comprises an amino acid sequence according to SEQ ID NO: 38; and
FR_{H}4 comprises an amino acid sequence according to SEQ ID NO: 39, and
wherein the antibody or antibody fragment comprises a light chain variable region (V_{L}) which comprises a framework region 1 (FR_{L}1), complementarity-determining region (CDR_{L}1), a FR_{L}2, a CDR_{L}2, a FR_{L}3, a CDR_{L}3, and a FR_{L}4, wherein
FR_{L}1 comprises an amino acid according to SEQ ID NO: 1;
CDR_{L}1 comprises an amino acid according to SEQ ID NOs: 2;
FR_{L}2 comprises an amino acid selected from SEQ ID NOs: 3, 15, and 29;
CDR_{L}2 comprises an amino acid sequence according to SEQ ID NO: 4;
FR_{L}3 comprises an amino acid sequence selected from SEQ ID NOs: 5 and 8;
CDR_{L}3 comprises an amino acid sequence selected from SEQ ID NOs: 6, 9, and 11; and
FR_{L}4 comprises an amino acid sequence selected from SEQ ID NOs: 7, 10, and 12.

4. The antibody or antibody fragment according to claim 1, wherein the antibody or antibody fragment comprises a heavy chain variable region (V_{H}) which comprises a framework region 1 (FR_{H}1), complementarity-determining region (CDR_{H}1), a FR_{H}2, a CDR_{H}2, a FR_{H}3, a CDR_{H}3, and a FR_{H}4, wherein
FR_{H}1 comprises an amino acid sequence according to SEQ ID NO: 40, 45, or 51;
CDR_{H}1 comprises an amino acid sequence according to SEQ ID NO: 41, 46, or 52, FR_{H}2 comprises an amino acid sequence according to SEQ ID NO: 35 or 55;
CDR_{H}2 comprises an amino acid sequence according to SEQ ID NO: 42 or 47;
FR_{H}3 comprises an amino acid sequence according to SEQ ID NO: 43, 48, or 53;
CDR_{H}3 comprises an amino acid sequence according to SEQ ID NO: 49, 44, or 54; and
FR_{H}4 comprises an amino acid sequence according to SEQ ID NO: 50, and
wherein the antibody or antibody fragment comprises a light chain variable region (V_{L}) which comprises a framework region 1 (FR_{L}1), complementarity-determining region (CDR_{L}1), a FR_{L}2, a CDR_{L}2, a FR_{L}3, a CDR_{L}3, and a FR_{L}4, wherein
FR_{L}1 comprises an amino acid according to SEQ ID NO: 13, 20, or 27;
CDR_{L}1 comprises an amino acid according to SEQ ID NO: 14, 21, or 28;
FR_{L}2 comprises an amino acid according to SEQ ID NO: 15, 22, or 29;
CDR_{L}2 comprises an amino acid sequence according to SEQ ID NO: 16, 23, or 30;
FR_{L}3 comprises an amino acid sequence according to SEQ ID NO: 17, 24, or 31;
CDR_{L}3 comprises an amino acid sequence according to SEQ ID NO: 18 or 25; and
FR_{L}4 comprises an amino acid sequence according to SEQ ID NO: 19 or 26.

5. The antibody or antibody fragment according to claim 1, wherein the antibody or antibody fragment comprises
a heavy chain variable region (V_{H}) according to SEQ ID NO: 62 and a light chain variable region (V_{L}) according to SEQ ID NO: 56,
a heavy chain variable region (V_{H}) according to SEQ ID NO: 63 and a light chain variable region (V_{L}) according to SEQ ID NO: 57,
a heavy chain variable region (V_{H}) according to SEQ ID NO: 64 and a light chain variable region (V_{L}) according to SEQ ID NO: 58,
a heavy chain variable region (V_{H}) according to SEQ ID NO: 65 and a light chain variable region (V_{L}) according to SEQ ID NO: 59,
a heavy chain variable region (V_{H}) according to SEQ ID NO: 66 and a light chain variable region (V_{L}) according to SEQ ID NO: 60, or
a heavy chain variable region (V_{H}) according to SEQ ID NO: 67 and a light chain variable region (V_{L}) according to SEQ ID NO: 61.

6-10. (canceled)

11. The antibody according to claim 1, wherein the antibody is a monoclonal antibody.

12. The antibody according to claim 1, wherein the antibody is a humanized or human antibody.

13. The antibody according to claim 1, wherein the antibody is an IgG type antibody, preferably an IgG1 antibody.

14. The antibody according to claim 1, wherein the antibody is a recombinant antibody.

15. The antibody according to claim 1, wherein the antibody comprises a wildtype heavy chain constant (Fc) region.

16. The antibody according to claim 15, wherein the Fc region comprises an amino acid sequence according to SEQ ID NO: 68.

17. (canceled)

18. The antibody according to claim 14, wherein the antibody comprises a heavy chain constant (Fc) region which comprises at least one amino acid substitution selected from L234A, L234S, L234G, L235A, L235G, L235S, L235T, G236R, D265C (according to EU numbering system).

19. (canceled)

20. The antibody according to claim 18, wherein the antibody comprises a heavy chain constant (Fc) region which comprises the amino acid substitutions L234A, L235A and D265C (according to EU numbering system).

21-25. (canceled)

26. The antibody according to claim 18, wherein the antibody comprises
a light chain (LC) amino acid sequence according to SEQ ID NO: 78 and a heavy chain (HC) amino acid sequence according to one of SEQ ID NOs: 79, 80, 81, 82, 83, or 84,
a light chain (LC) amino acid sequence according to SEQ ID NO: 85 and a heavy chain (HC) amino acid sequence according to one of SEQ ID NOs: 86, 87, 88, 89, 90, or 91,
a light chain (LC) amino acid sequence according to SEQ ID NO: 92 and a heavy chain (HC) amino acid sequence according to one of SEQ ID NOs: 93, 94, 95, 96, 97, or 98,
a light chain (LC) amino acid sequence according to SEQ ID NO: 99 and a heavy chain (HC) amino acid sequence according to one of SEQ ID NOs: 100, 101, 102, 103, 104, or 105,
a light chain (LC) amino acid sequence according to SEQ ID NO: 106 and a heavy chain (HC) amino acid sequence according to one of SEQ ID NOs: 107, 108, 109, 110, 111, or 112, or
a light chain (LC) amino acid sequence according to SEQ ID NO: 113 and a heavy chain (HC) amino acid sequence according to one of SEQ ID NOs: 114, 115, 116, 117, 118, or 119.

27-31. (canceled)

32. The antibody according to claim 1, wherein said antibody specifically binds to an epitope comprised inamino acids 24-430 of SEQ ID NO: 76 or amino acids 23-430 of SEQ ID NO:77.

33. (canceled)

34. A conjugate comprising (i) the antibody according to claim 1, (ii) at least one toxin, and (iii) at least one linker connecting said antibody moiety with said at least one toxin, wherein said antibody specifically binds to human GUCY2C and wherein said at least one toxin is an amatoxin.

35. The conjugate according to claim 34, wherein said linker is connected to said antibody via any of the naturally occurring cysteine residues of said antibody.

36. The conjugate according to claim 34, wherein said linker is connected via a disulfide linkage between Cys265 of said antibody and said linker (numbering according to the EU numbering system).

37-40. (canceled)

41. The conjugate according to claim 34, wherein the linker is a Cathepsin B-cleavable linker comprising a di-peptide selected from Val-Cit, Ala-Val, or Phe-Lys, Val-Lys, Ala-Lys, Phe-Cit, Leu-Cit, Ile-Cit, Phe-Arg, or Trp-Cit.

42. Conjugate according to claim 34, wherein said linker is connected to said amatoxin via (i) the γC-atom of amatoxin amino acid 1, or (ii) the δ-C-atom of amatoxin amino acid 3, or (iii) the 6′-C-atom of amatoxin amino acid 4.

43. The conjugate according to claim 34, wherein said amatoxin comprises (i) an amino acid 4 with a 6′-deoxy position and (ii) an amino acid 8 with an S-deoxy position.

44. The conjugate according to claim 34, wherein said conjugate comprises any of the following amatoxin-linker compounds of formulas (I) to (XI)

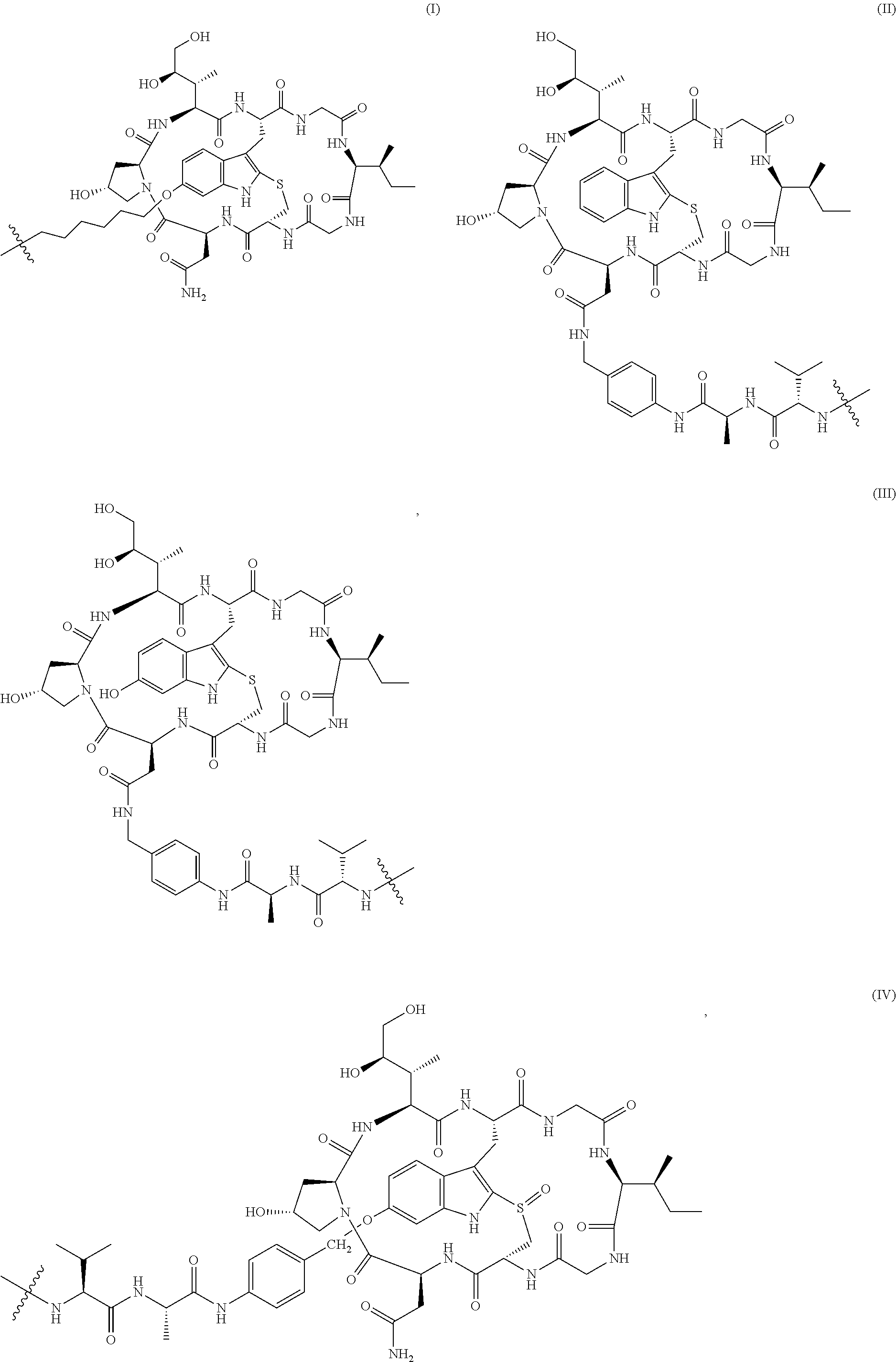

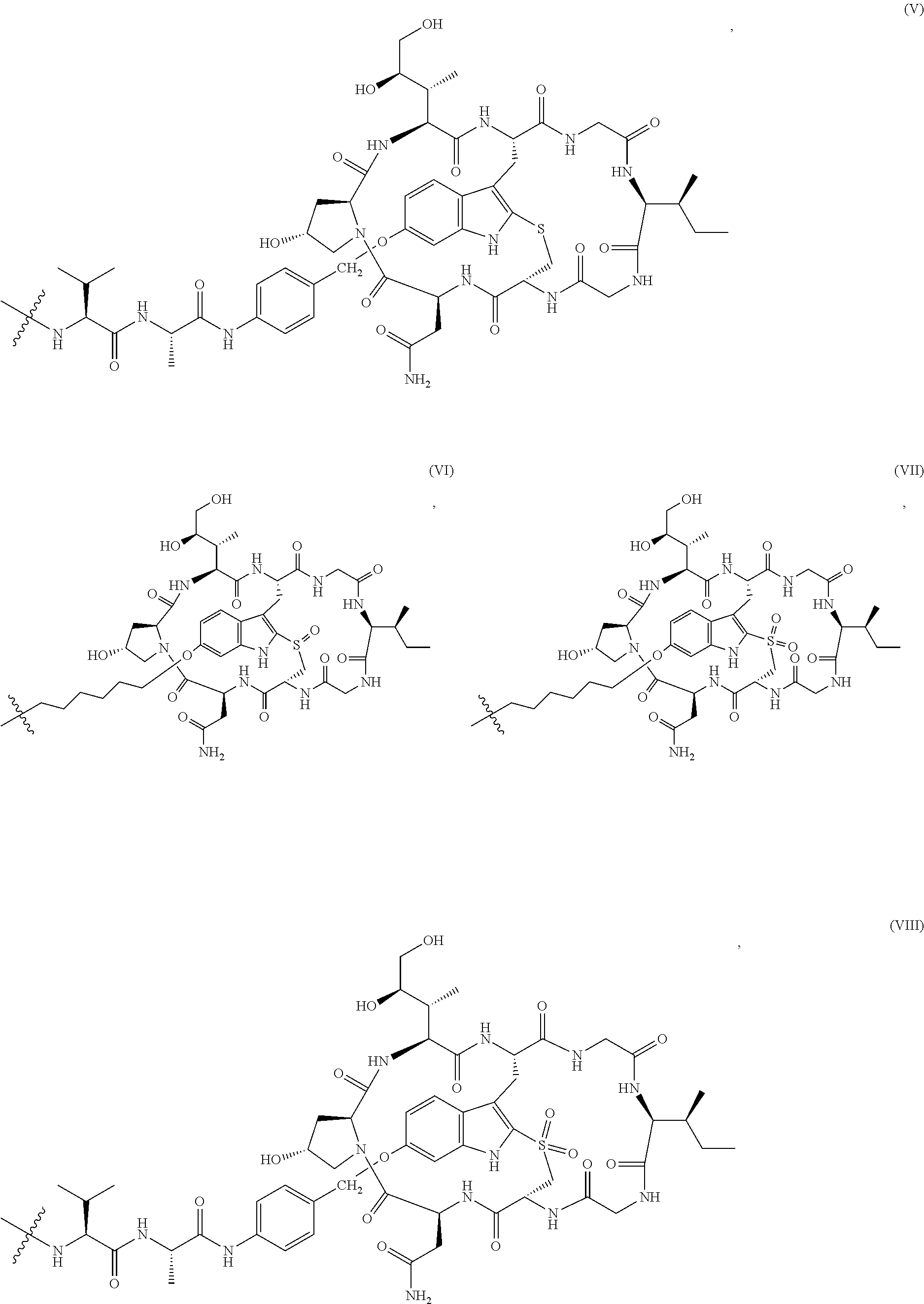

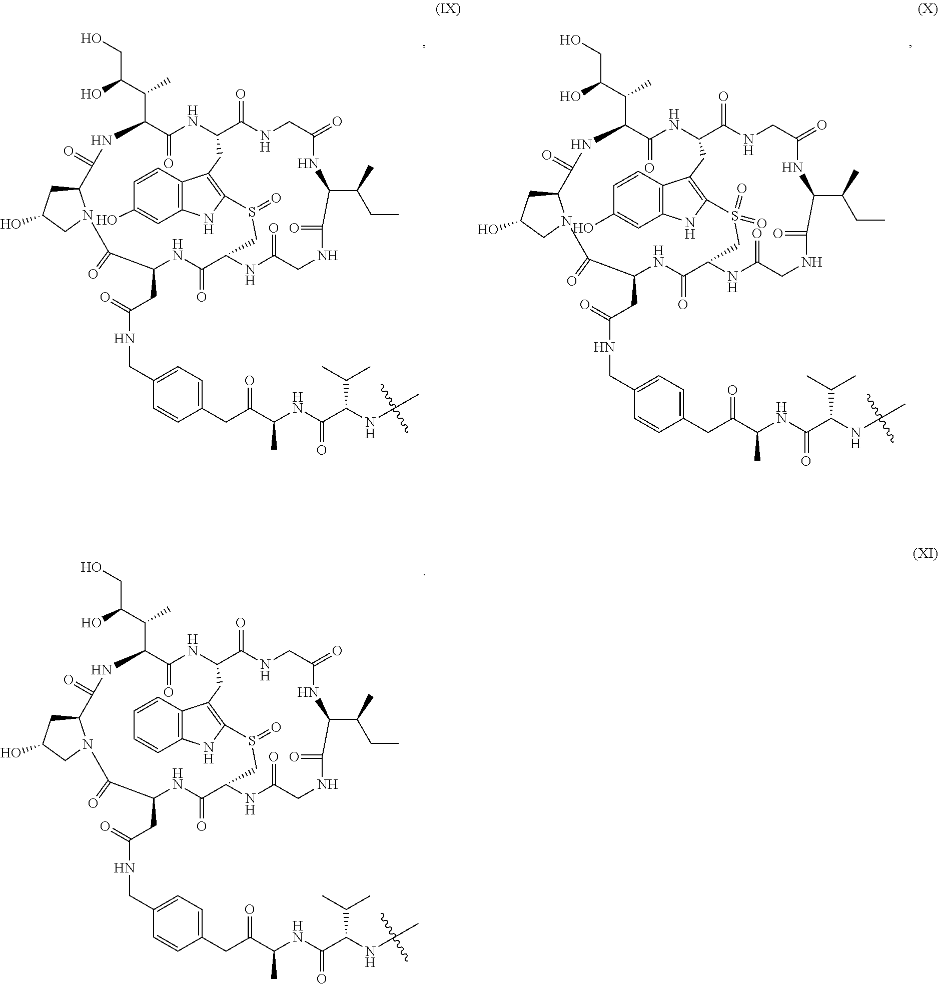

45. The conjugate according to claim 34, wherein said conjugate comprises said antibody conjugated to amatoxin linker moieties via a thioether linkage according to any one of formula (XII) to (XXII): 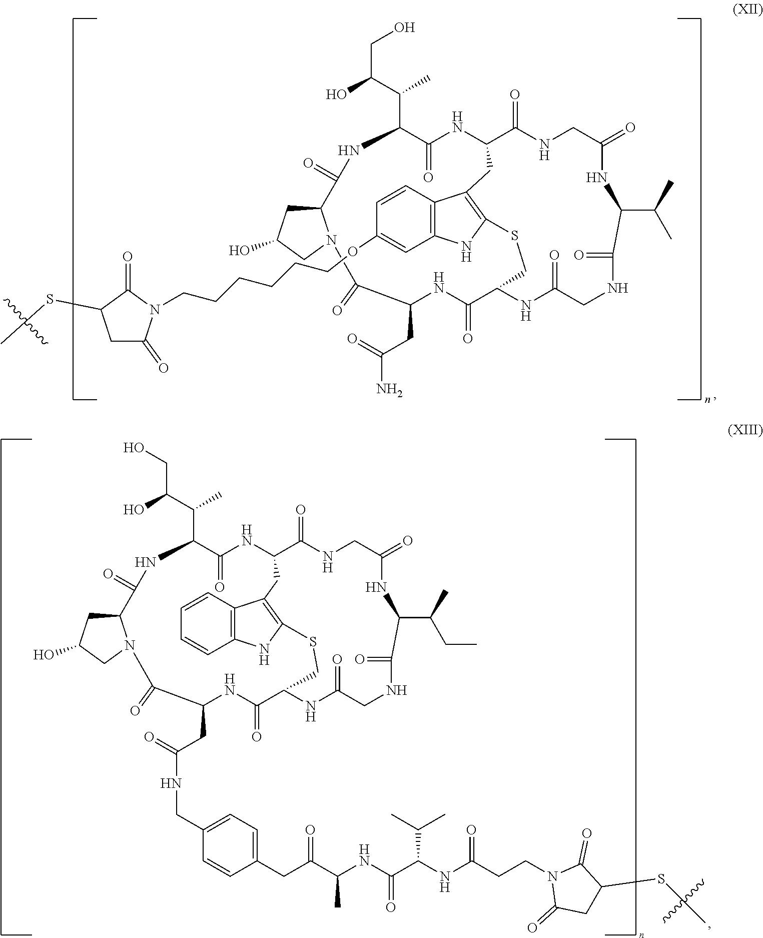 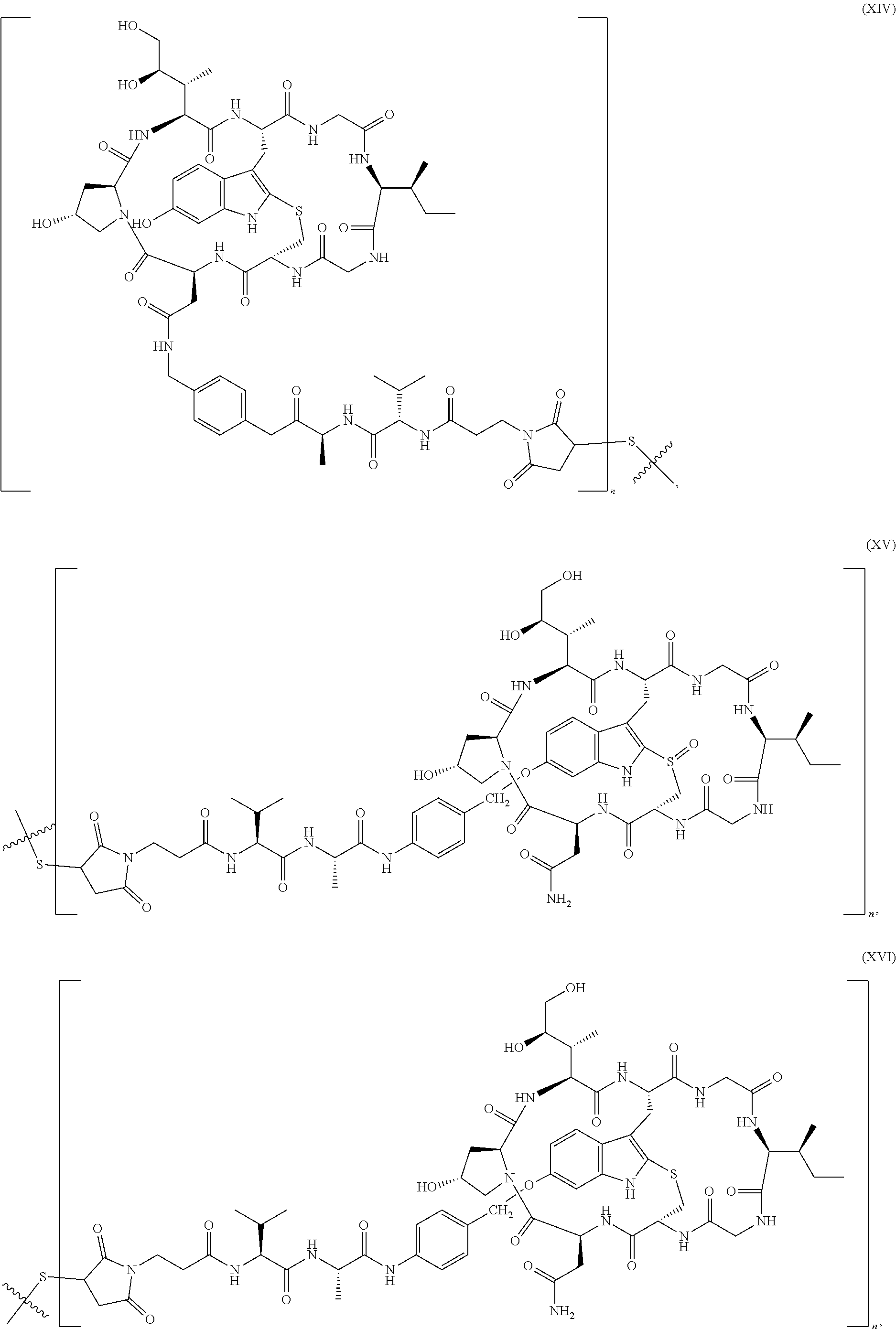 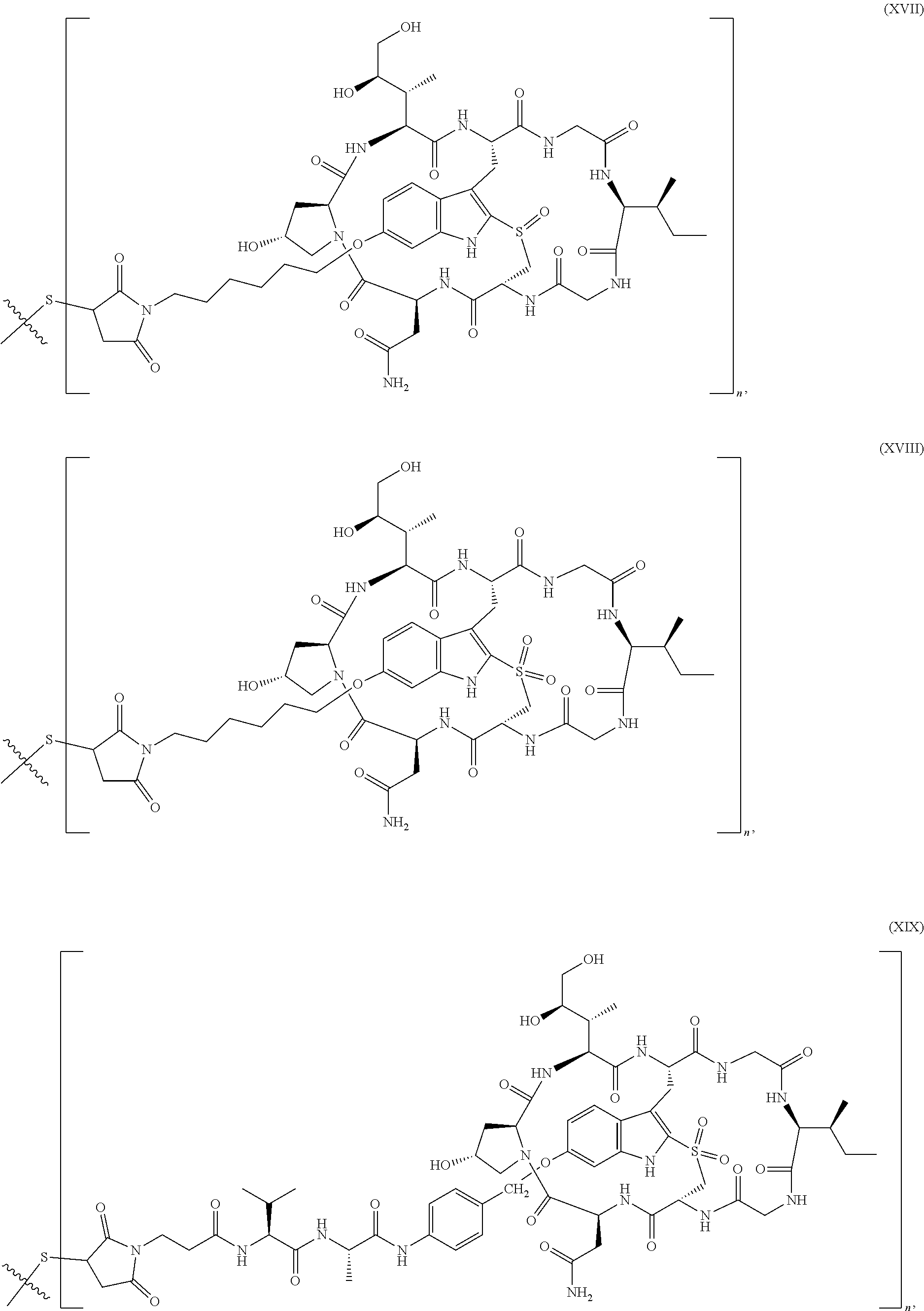 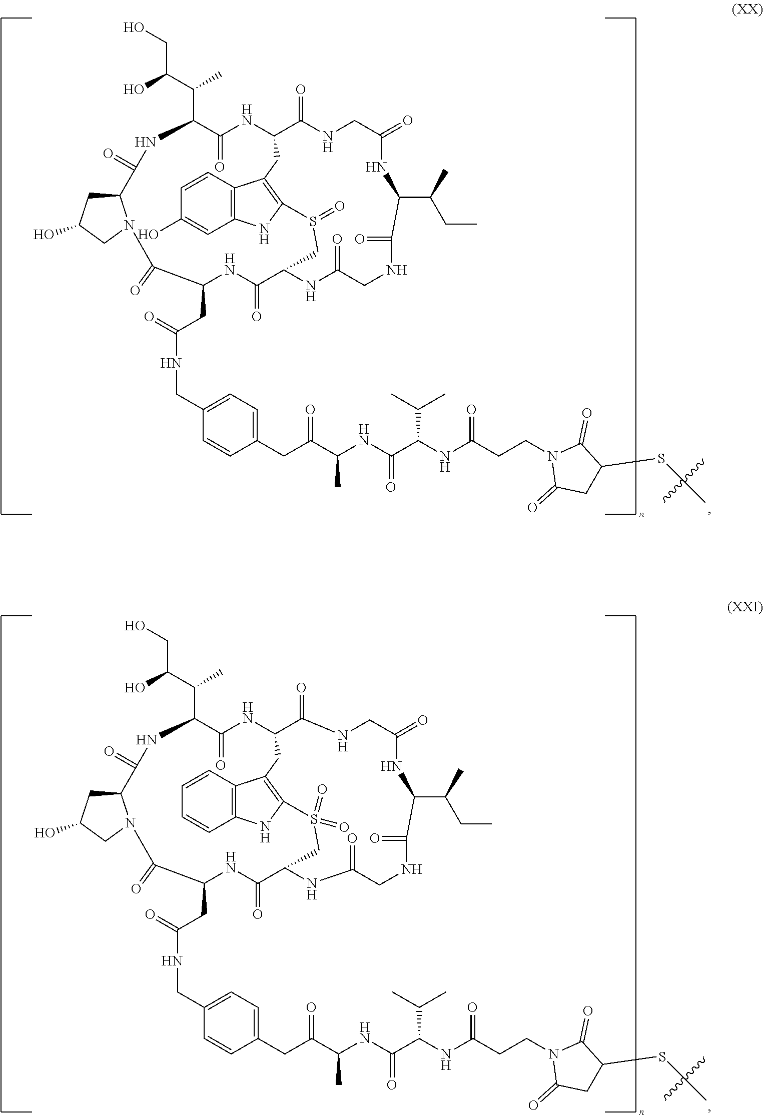 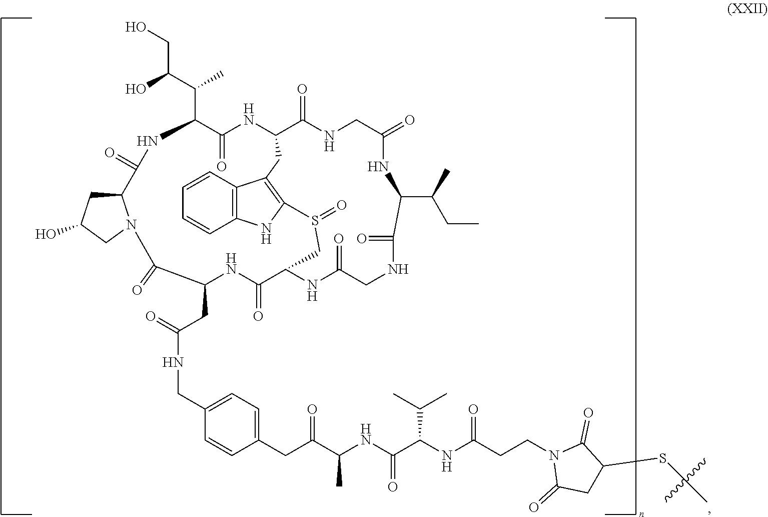
wherein said amatoxin linker moieties are coupled to the thiol groups of cysteine residues of the antibody, and wherein n is about 1 to about 8.

46. The conjugate according to claim 45, wherein said amatoxin linker moieties are coupled to the thiol groups of cysteine residues of the antibody, and wherein n is from about 1 to about 2.

47-119. (canceled)

120. A polynucleotide encoding a polypeptide comprising the amino acid sequence of any of SEQ ID NOs: 56-67.

121. An expression vector comprising a polynucleotide according to claim 120.

122. A host cell comprising a polynucleotide according to claim 120.

123. (canceled)

124. (canceled)

125. A pharmaceutical composition comprising the conjugate according to claim 34.

126. (canceled)

127. A composition comprising at least one immune checkpoint inhibitor, and at least one conjugate according to claim 34.

128.-134. (canceled)

135. A method of treating a patient afflicted with a solid tumor which expresses amino acids 24-430 of SEQ ID NO: 76 or a fragment thereof on its surface, wherein the method comprises administering to said patient a therapeutically effective amount of the conjugate according to claim 34.

136. A method of treating a patient afflicted with gastrointestinal cancer, wherein the method comprises administering to said patient a therapeutically effective amount of the conjugate according to claim 34.

137-141. (canceled)


SEQUENCE LISTING (123 sequences)

SEQ ID NO: 1 - protein, 23 aa, synthetic construct
DIRMTQSPSSLSASAGDRVTITC

SEQ ID NO: 2 - protein, 11 aa, synthetic construct
RASQSISSYLN

SEQ ID NO: 3 - protein, 15 aa, synthetic construct
WYQQKPGKAPKLLIY

SEQ ID NO: 4 - protein, 7 aa, synthetic construct
AASSLQS

SEQ ID NO: 5 - protein, 32 aa, synthetic construct
GVPSRFSGSGSGTDFTLTISSLQPEDFATYYC

SEQ ID NO: 6 - protein, 9 aa, synthetic construct
QQSYSTPLT

SEQ ID NO: 7 - protein, 10 aa, synthetic construct
FGGGTKLEIK

SEQ ID NO: 8 - protein, 32 aa, synthetic construct
GVPSRFSGSGSGTDFTLAISSLQPEDFATYYC

SEQ ID NO: 9 - protein, 9 aa, synthetic construct
QQSYSTPWT

SEQ ID NO: 10 - protein, 10 aa, synthetic construct
FGQGTKVDIK

SEQ ID NO: 11 - protein, 9 aa, synthetic construct
QQSYSTPYT

SEQ ID NO: 12 - protein, 10 aa, synthetic construct
FGQGTKVEIK

SEQ ID NO: 13 - protein, 22 aa, synthetic construct
QSVLTQPPSASGTPGQRVTISC

SEQ ID NO: 14 - protein, 13 aa, synthetic construct
SGSSSNIGSNTVN

SEQ ID NO: 15 - protein, 15 aa, synthetic construct
WYQQLPGTAPRLLIY

SEQ ID NO: 16 - protein, 7 aa, synthetic construct
SNRQRPS

SEQ ID NO: 17 - protein, 31 aa, synthetic construct
VPDRFSGARSGTSASLAITGLQPEDEADYYC

SEQ ID NO: 18 - protein, 11 aa, synthetic construct
ATWDVGLRGRV

SEQ ID NO: 19 - protein, 10 aa, synthetic construct
FGGGTKLTVL

SEQ ID NO: 20 - protein, 22 aa, synthetic construct
QSGLTQPRSVSGSPGQSVTISC

SEQ ID NO: 21 - protein, 14 aa, synthetic construct
TGTSSDVGGYKYVS

SEQ ID NO: 22 - protein, 15 aa, synthetic construct
WYQQHPGKAPKLKIY

SEQ ID NO: 23 - protein, 7 aa, synthetic construct
DVSKRPS

SEQ ID NO: 24 - protein, 32 aa, synthetic construct
GVPDRFSGSKSGNTASLTISGLQAEDEADYYC

SEQ ID NO: 25 - protein, 10 aa, synthetic construct
TSYTSSNTVV

SEQ ID NO: 26 - protein, 10 aa, synthetic construct
FGGGTKVTVL

SEQ ID NO: 27 - protein, 22 aa, synthetic construct
QSALTQPASVSGSPGQSITISC

SEQ ID NO: 28 - protein, 14 aa, synthetic construct
TGTSSDVGSYNLVS

SEQ ID NO: 29 - protein, 15 aa, synthetic construct
WYQQHPGKAPKLMIY

SEQ ID NO: 30 - protein, 7 aa, synthetic construct
EGSKRPS

SEQ ID NO: 31 - protein, 32 aa, synthetic construct
GVSNRFSASKSGNTASLTISGLQTEDEADYYC

SEQ ID NO: 32 - protein, 10 aa, synthetic construct
SSYVPRSSLV

SEQ ID NO: 33 - protein, 30 aa, synthetic construct
QVQLVQSGAEVKKPGASVKVSCKASGYIFI

SEQ ID NO: 34 - protein, 5 aa, synthetic construct
SSAMH

SEQ ID NO: 35 - protein, 14 aa, synthetic construct
WVRQAPGQRLEWMG

SEQ ID NO: 36 - protein, 17 aa, synthetic construct
LINPGNGNTKYSQKFQG

SEQ ID NO: 37 - protein, 32 aa, synthetic construct
RVTITRDTSASTAYMELSSLRSEDTAVYYCAR

SEQ ID NO: 38 - protein, 10 aa, synthetic construct
AYFRGLGFDI

SEQ ID NO: 39 - protein, 11 aa, synthetic construct
WGQGTLVTVSS

SEQ ID NO: 40 - protein, 30 aa, synthetic construct
EVQLVQSGAEVKKPGASVKVSCKASGYTFT

SEQ ID NO: 41 - protein, 5 aa, synthetic construct
SYYMH

SEQ ID NO: 42 - protein, 17 aa, synthetic construct
IINPSGGSTSYAQKFQG

SEQ ID NO: 43 - protein, 32 aa, synthetic construct
RVTMTRDTSTSTVYMELSSLRSEDTAVYYCAR

SEQ ID NO: 44 - protein, 10 aa, synthetic construct
VRQWPLASDY

SEQ ID NO: 45 - protein, 30 aa, synthetic construct
QVQLVESGAEVKKPGASVKVSCKASGYTFT

SEQ ID NO: 46 - protein, 5 aa, synthetic construct
SYAIS

SEQ ID NO: 47 - protein, 17 aa, synthetic construct
RIIPILGIANYAQKFQG

SEQ ID NO: 48 - protein, 32 aa, synthetic construct
RVTITADKSTSTAYMELSRLRSDDTAVYYCAI

SEQ ID NO: 49 - protein, 10 aa, synthetic construct
DSSGWMTFDY

SEQ ID NO: 50 - protein, 11 aa, synthetic construct
WGQGTMVTVSS

SEQ ID NO: 51 - protein, 30 aa, synthetic construct
QVQLVQSGAEVKKPGSSVKVSCKASGGTFS

SEQ ID NO: 52 - protein, 5 aa, synthetic construct
TYTIN

SEQ ID NO: 53 - protein, 32 aa, synthetic construct
RVTITADKSTSTAYMELSSLRSEDTAVYYCAR

SEQ ID NO: 54 - protein, 13 aa, synthetic construct
DTPRLRSSYYMDV

SEQ ID NO: 55 - protein, 14 aa, synthetic construct
WVRQAPGQGLEWMG

SEQ ID NO: 56 - protein, 107 aa, synthetic construct
DIQMTQSPSSLSASAGDRVTITCRASQSISSYLNWYQQKPGKAPKLLIYAASSLQSGVPSRFSGSGSGTDFTLAISSLQPEDFATYYCQQSYSTPWTFGQGTKVDIK

SEQ ID NO: 57 - protein, 107 aa, synthetic construct
DIRMTQSPSSLSASAGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 58 - protein, 107 aa, synthetic construct
DIQMTQSPSSLSASAGDRVTITCRASQSISSYLNWYQQKPGKAPKLLIYAASSLQSGVPSRFSGSGSGTDFTLAISSLQPEDFATYYCQQSYSTPYTFGQGTKVEIK

SEQ ID NO: 59 - protein, 110 aa, synthetic construct
QSGLTQPRSVSGSPGQSVTISCTGTSSDVGGYKYVSWYQQHPGKAPKLKIYDVSKRPSGVPDRFSGSKSGNTASLTISGLQAEDEADYYCTSYTSSNTVVFGGGTKVTVL

SEQ ID NO: 60 - protein, 110 aa, synthetic construct
QSVLTQPPSASGTPGQRVTISCSGSSSNIGSNTVNWYQQLPGTAPRLLIYSNRQRPSGVPDRFSGARSGTSASLAITGLQPEDEADYYCATWDVGLRGRVFGGGTKLTVL

SEQ ID NO: 61 - protein, 110 aa, synthetic construct
QSALTQPASVSGSPGQSITISCTGTSSDVGSYNLVSWYQQHPGKAPKLMIYEGSKRPSGVSNRFSASKSGNTASLTISGLQTEDEADYYCSSYVPRSSLVFGGGTKLTVL

SEQ ID NO: 62 - protein, 119 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSS

SEQ ID NO: 63 - protein, 119 aa, synthetic construct; identical to SEQ ID NO: 62

SEQ ID NO: 64 - protein, 119 aa, synthetic construct; identical to SEQ ID NO: 62

SEQ ID NO: 65 - protein, 119 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSS

SEQ ID NO: 66 - protein, 119 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSS

SEQ ID NO: 67 - protein, 122 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSS

SEQ ID NO: 68 - protein, 330 aa, synthetic construct [sequence omitted]

SEQ ID NO: 69 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAARGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGSRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESGRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 1073 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 77 - protein, 1073 aa, synthetic construct
MKTLLLDLVLWSLLFQPEWLYLTSQVSQNCHNGSYEISVLMMDNSAFAEPLENVEDAVNEGLEIVRGRLQNAGLNVTVNASFMYSDGLIHNSGDCRSSTCEGLDLLRKISNAKRMGCVLMGPSCTYSTFQMYLDTELSYPMISAGSFGLSCDYKETLTRLMSPARKLTYFLVNFWKTNDLPFKTYSWSTSYVYKNGTESEDCFWYLNALEASVSYFSHELSFKLVLRQDKEFQDILMDHNRKSNVIVMCGDPEFLYKLKGDRAVAEDIVIILVDLFNDQYFEDNVTAPDYMKNVLVLTRSPGNSLLNSSFSRNLSPTKRDFALAYLNGILLFGHMLKTFLENGENITTPKFAHAFRNLTFEGYDGPVTLDDWGDVDSTMVLLYTSVDTKKYKVLLTYDTHVNQTNPVDMSPTFTWKNSKLPNDITDRGPQILMIAVFTLTGAVVLLLLVALLMLRKYKKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANVSQKGDVYSYGIIAQEIILRKETFYTSSCRDRNEKIFRVENSNGMKPFRPDLFLETAEEKELEVYLLVKSCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 78 - protein, 214 aa, synthetic construct
DIQMTQSPSSLSASAGDRVTITCRASQSISSYLNWYQQKPGKAPKLLIYAASSLQSGVPSRFSGSGSGTDFTLAISSLQPEDFATYYCQQSYSTPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAARGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGSRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESGRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 214 aa, synthetic construct
DIRMTQSPSSLSASAGDRVTITCRASQSISSYLNWYQQKPGKAPKLLIYAASSLQSGVPSRFSGSGSGTDFTLTISSLQPEDFATYYCQQSYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 449 aa, synthetic construct; identical to SEQ ID NO: 79

SEQ ID NO: 87 - protein, 449 aa, synthetic construct; identical to SEQ ID NO: 80

SEQ ID NO: 88 - protein, 449 aa, synthetic construct; identical to SEQ ID NO: 81

SEQ ID NO: 89 - protein, 449 aa, synthetic construct; identical to SEQ ID NO: 82

SEQ ID NO: 90 - protein, 449 aa, synthetic construct; identical to SEQ ID NO: 83

SEQ ID NO: 91 - protein, 449 aa, synthetic construct; identical to SEQ ID NO: 84

SEQ ID NO: 92 - protein, 214 aa, synthetic construct
DIQMTQSPSSLSASAGDRVTITCRASQSISSYLNWYQQKPGKAPKLLIYAASSLQSGVPSRFSGSGSGTDFTLAISSLQPEDFATYYCQQSYSTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 449 aa, synthetic construct; identical to SEQ ID NO: 79

SEQ ID NO: 94 - protein, 449 aa, synthetic construct; identical to SEQ ID NO: 80

SEQ ID NO: 95 - protein, 449 aa, synthetic construct; identical to SEQ ID NO: 81

SEQ ID NO: 96 - protein, 449 aa, synthetic construct; identical to SEQ ID NO: 82

SEQ ID NO: 97 - protein, 449 aa, synthetic construct; identical to SEQ ID NO: 83

SEQ ID NO: 98 - protein, 449 aa, synthetic construct; identical to SEQ ID NO: 84

SEQ ID NO: 99 - protein, 216 aa, synthetic construct
QSGLTQPRSVSGSPGQSVTISCTGTSSDVGGYKYVSWYQQHPGKAPKLKIYDVSKRPSGVPDRFSGSKSGNTASLTISGLQAEDEADYYCTSYTSSNTVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 100 - protein, 449 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 449 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 449 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAARGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 449 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGSRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 449 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 449 aa, synthetic construct
QVQLVESGAEVKKPGASVKVSCKASGYTFTSYAISWVRQAPGQGLEWMGRIIPILGIANYAQKFQGRVTITADKSTSTAYMELSRLRSDDTAVYYCAIDSSGWMTFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESGRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 216 aa, synthetic construct
QSVLTQPPSASGTPGQRVTISCSGSSSNIGSNTVNWYQQLPGTAPRLLIYSNRQRPSGVPDRFSGARSGTSASLAITGLQPEDEADYYCATWDVGLRGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 107 - protein, 449 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 449 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 449 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAARGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 449 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGSRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 449 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 449 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVRQWPLASDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESGRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 216 aa, synthetic construct
QSALTQPASVSGSPGQSITISCTGTSSDVGSYNLVSWYQQHPGKAPKLMIYEGSKRPSGVSNRFSASKSGNTASLTISGLQTEDEADYYCSSYVPRSSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAARGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEGSRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESGRGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 17 aa, synthetic construct
RIIPVLGIANYAQKFQG

SEQ ID NO: 121 - protein, 23 aa, synthetic construct
DIQMTQSPSSLSASAGDRVTITC

SEQ ID NO: 122 - protein, 8 aa, synthetic construct
  MOD_RES 3: note = 4Hyp
  MOD_RES 4: note = Dihydroxyisoleucine
CDPIWGIG

SEQ ID NO: 123 - protein, 8 aa, synthetic construct
  VARIANT 3: note = X is isoleucine, hydroxyisoleucine, or dihydroxyisoleucine.
  VARIANT 4: note = X is tryptophan or hydroxytryptophan.
BPXXGIGC